SEQUENCE LISTING (92 sequences)

SEQ ID NO: 1 - skipped

SEQ ID NO: 2 - protein, 17 aa, synthetic construct
VINPGGDNTDYTYAVKG

SEQ ID NO: 3 - protein, 16 aa, synthetic construct
GRTFGGTWMDRNKWKY

SEQ ID NO: 4 - protein, 5 aa, synthetic construct
SGCMG

SEQ ID NO: 5 - protein, 17 aa, synthetic construct
  VARIANT 14: note = S or T
TIYTRNDFTYYSDXVKG

SEQ ID NO: 6 - protein, 22 aa, synthetic construct
GSPFRYAGAWNVCGLKEEDYNY

SEQ ID NO: 7 - protein, 5 aa, synthetic construct
ANCVG

SEQ ID NO: 8 - protein, 17 aa, synthetic construct
AINTGGETATYADFVKG

SEQ ID NO: 9 - protein, 19 aa, synthetic construct
VGTRKYWDECSLSQHAYKN

SEQ ID NO: 10 - protein, 5 aa, synthetic construct
SYAMD

SEQ ID NO: 11 - protein, 16 aa, synthetic construct
GINSAGSPSYEASVKG

SEQ ID NO: 12 - protein, 10 aa, synthetic construct
CRDFRSPYCR

SEQ ID NO: 13 - skipped

SEQ ID NO: 14 - protein, 16 aa, synthetic construct
SISSDGNSDYLDSVKG

SEQ ID NO: 15 - protein, 19 aa, synthetic construct
NGAGHLVLRPLCGSNAVDA

SEQ ID NO: 16 - protein, 5 aa, synthetic construct
GTTMG

SEQ ID NO: 17 - protein, 16 aa, synthetic construct
DISSDGNTYYSDSVKG

SEQ ID NO: 18 - protein, 18 aa, synthetic construct
AEFRLLLRPLCRQDDYGY

SEQ ID NO: 19 - protein, 5 aa, synthetic construct
YKYLA

SEQ ID NO: 20 - protein, 17 aa, synthetic construct
GLNTGGGSAYYADSVKG

SEQ ID NO: 21 - protein, 22 aa, synthetic construct
RRDYIHMTNIQALGLRPQWFSD

SEQ ID NO: 22 - protein, 5 aa, synthetic construct
SNFMA

SEQ ID NO: 23 - protein, 17 aa, synthetic construct
TITTGAGNTAYADSVKG

SEQ ID NO: 24 - protein, 13 aa, synthetic construct
GRGGFWRPAEYDF

SEQ ID NO: 25 - protein, 123 aa, synthetic construct
QVQLVESGGGSVQAGGSLRLSCVVSGYTYSSVAWFRQTPGKEREGVAVINPGGDNTDYTYAVKGRFTISRDSAKSTVFLQMNNLESEDTAMYYCAAGRTFGGTWMDRNKWKYWGQGTLVTVSS

SEQ ID NO: 26 - protein, 131 aa, synthetic construct
QVQLVESGGDSVQAGGSLRLSCAASAHAYSSGCMGWFRQAPGKGREGVATIYTRNDFTYYSDSVKGRFTISRDDAKNTVYLQVNSLKAEDTAMYYCAAGSPFRYAGAWNVCGLKEEDYNYWGQGTLVTVSS

SEQ ID NO: 27 - protein, 128 aa, synthetic construct
QVQLVESGGGLVQPGGSLRLSCAASGYSYSANCVGWFYQAPGKEREGVAAINTGGETATYADFVKGRFTISQDYAKNTVYLQMNSLKSEDTGMYYCAAVGTRKYWDECSLSQHAYKNWGQGTLVTVSS

SEQ ID NO: 28 - protein, 118 aa, synthetic construct
EVQLVESGGGLVQPGGSLRLSCVASGFTFKSYAMDWVRQAPGKGLEWIAGINSAGSPSYEASVKGRFTISRDNAENTLYLQLNSLKTEDTAMYYCTKCRDFRSPYCRPGQGTLVTVSS

SEQ ID NO: 29 - protein, 124 aa, synthetic construct
EVQLVESGGGSVQAGGSLRLSCTASGFTFDMGWYRQAPGDECELVSSISSDGNSDYLDSVKGRFTISQDNAKNTVYLQMNSLKPEDTAVYYCAANGAGHLVLRPLCGSNAVDAWGQGTLVTVSS

SEQ ID NO: 30 - protein, 126 aa, synthetic construct
QVQLVESGGDSVQAGGSLRLSCIASGFTFDGTTMGWYRQAPGDECELVSDISSDGNTYYSDSVKGRFTISQGNAKNTVYLQMNSLTPEDTAVYYCAAAEFRLLLRPLCRQDDYGYWGQGTLVTVSS

SEQ ID NO: 31 - protein, 131 aa, synthetic construct
QVQLQESGGGLVQPGGSLRLSCAASGTTGDYKYLAWFRQGPGKEREGIAGLNTGGGSAYYADSVKGRFTISQDNVKRLLYLQLNNLKPEDTAMYYCAARRDYIHMTNIQALGLRPQWFSDWGQGTQVTVSS

SEQ ID NO: 32 - protein, 122 aa, synthetic construct
EVQLVESGGGSVQAQGSLRLSCTVSGSTYSSNFMAWFRQAPGKEREWVATITTGAGNTAYADSVKGRFTISEDNAKRTAYLQMNSLKPEDTAVYYCAAGRGGFWRPAEYDFWGQGTQVTVSS

SEQ ID NO: 33 - protein, 131 aa, synthetic construct
QVQLLESGGGSVQAGGSLRLSCAASGFTFSSGCMGWFRQAPGKGREGVATIYTRNDFTYYSDSVKGRFTISRDNSKNTVYLQMNSLRAEDTAVYYCAAGSPFRYAGAWNVCGLKEEDYNYWGQGTLVTVSS

SEQ ID NO: 34 - protein, 131 aa, synthetic construct
QVQLLESGGGSVQAGGSLRLSCAASGHTYSSGCMGWFRQAPGKGREGVATIYTRNDFTYYSDSVKGRFTISRDNSKNTVYLQMNSLRAEDTAVYYCAAGSPFRYAGAWNVCGLKEEDYNYWGQGTLVTVSS

SEQ ID NO: 35 - protein, 131 aa, synthetic construct
QVQLLESGGGSVQAGGSLRLSCAASGHTYSSGCMGWFRQAPGKGREGVATIYTRNDFTYYSDTVKGRFTISRDNSKNTVYLQMNSLRAEDTAVYYCAAGSPFRYAGAWNVCGLKEEDYNYWGQGTLVTVSS

SEQ ID NO: 36 - protein, 128 aa, synthetic construct
QVQLVESGGGSVQPGGSLRLSCAASGYSYSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSS

SEQ ID NO: 37 - protein, 128 aa, synthetic construct
QVQLVESGGGSVQPGGSLRLSCAASGFTFSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSS

SEQ ID NO: 38 - protein, 122 aa, synthetic construct
EVQLVESGGGSVQAGGSLRLSCAVSGFTVSSNFMAWFRQAPGKGREWVSTITTGAGNTAYADSVKGRFTISEDNSKNTLYLQMNSLRAEDTAVYYCAAGRGGFWRPAEYDFWGQGTTVTVSS

SEQ ID NO: 39 - protein, 122 aa, synthetic construct
EVQLVESGGGSVQAGGSLRLSCAVSGFTVSSNFMAWFRQAPGKGREWVSTITTGAGNTAYADSVKGRFTISEDNSKNTAYLQMNSLRAEDTAVYYCAAGRGGFWRPAEYDFWGQGTTVTVSS

SEQ ID NO: 40 - protein, 131 aa, synthetic construct
  VARIANT 5: note = V or L
  VARIANT 10: note = D or G
  VARIANT 26: note = A or G
  VARIANT 27: note = H or F
  VARIANT 28: note = A or T
  VARIANT 29: note = Y or F
  VARIANT 63: note = S or T
  VARIANT 74: note = D or N
  VARIANT 75: note = A or S
  VARIANT 83: note = V or M
  VARIANT 87: note = K or R
  VARIANT 93: note = M or V
QVQLXESGGXSVQAGGSLRLSCAASXXXXSSGCMGWFRQAPGKGREGVATIYTRNDFTYYSDXVKGRFTISRDXXKNTVYLQXNSLXAEDTAXYYCAAGSPFRYAGAWNVCGLKEEDYNYWGQGTLVTVSS

SEQ ID NO: 41 - protein, 128 aa, synthetic construct
  VARIANT 11: note = L or S
  VARIANT 27: note = Y or F
  VARIANT 28: note = S or T
  VARIANT 29: note = Y or F
  VARIANT 38: note = Y or R
  VARIANT 44: note = E or G
  VARIANT 49: note = A or S
  VARIANT 72: note = Q or R
  VARIANT 74: note = Y or N
  VARIANT 87: note = K or R
  VARIANT 88: note = S or A
  VARIANT 92: note = G or A
  VARIANT 93: note = M or V
QVQLVESGGGXVQPGGSLRLSCAASGXXXSANCVGWFXQAPGKXREGVXAINTGGETATYADFVKGRFTISXDXAKNTVYLQMNSLXXEDTXXYYCAAVGTRKYWDECSLSQHAYKNWGQGTLVTVSS

SEQ ID NO: 42 - protein, 122 aa, synthetic construct
  VARIANT 15: note = Q or G
  VARIANT 23: note = T or A
  VARIANT 27: note = S or F
  VARIANT 29: note = Y or V
  VARIANT 44: note = E or G
  VARIANT 49: note = A or S
  VARIANT 75: note = A or S
  VARIANT 77: note = R or N
  VARIANT 79: note = A or L
  VARIANT 87: note = K or R
  VARIANT 88: note = P or A
  VARIANT 117: note = Q or T
EVQLVESGGGSVQAXGSLRLSCXVSGXTXSSNFMAWFRQAPGKXREWVXTITTGAGNTAYADSVKGRFTISEDNXKXTXYLQMNSLXXEDTAVYYCAAGRGGFWRPAEYDFWGQGTXVTVSS

SEQ ID NO: 43 - protein, 375 aa, synthetic construct
QVQLVESGGGSVQAGGSLRLSCVVSGYTYSSVAWFRQTPGKEREGVAVINPGGDNTDYTYAVKGRFTISRDSAKSTVFLQMNNLESEDTAMYYCAAGRTFGGTWMDRNKWKYWGQGTLVTVSSEPKIPQPQPKPQPQPQPQPKPQPKPEPECTCPKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 383 aa, synthetic construct
QVQLVESGGDSVQAGGSLRLSCAASAHAYSSGCMGWFRQAPGKGREGVATIYTRNDFTYYSDSVKGRFTISRDDAKNTVYLQVNSLKAEDTAMYYCAAGSPFRYAGAWNVCGLKEEDYNYWGQGTLVTVSSEPKIPQPQPKPQPQPQPQPKPQPKPEPECTCPKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 380 aa, synthetic construct
QVQLVESGGGLVQPGGSLRLSCAASGYSYSANCVGWFYQAPGKEREGVAAINTGGETATYADFVKGRFTISQDYAKNTVYLQMNSLKSEDTGMYYCAAVGTRKYWDECSLSQHAYKNWGQGTLVTVSSEPKIPQPQPKPQPQPQPQPKPQPKPEPECTCPKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 370 aa, synthetic construct
EVQLVESGGGLVQPGGSLRLSCVASGFTFKSYAMDWVRQAPGKGLEWIAGINSAGSPSYEASVKGRFTISRDNAENTLYLQLNSLKTEDTAMYYCTKCRDFRSPYCRPGQGTLVTVSSEPKIPQPQPKPQPQPQPQPKPQPKPEPECTCPKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 376 aa, synthetic construct
EVQLVESGGGSVQAGGSLRLSCTASGFTFDMGWYRQAPGDECELVSSISSDGNSDYLDSVKGRFTISQDNAKNTVYLQMNSLKPEDTAVYYCAANGAGHLVLRPLCGSNAVDAWGQGTLVTVSSEPKIPQPQPKPQPQPQPQPKPQPKPEPECTCPKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 378 aa, synthetic construct
QVQLVESGGDSVQAGGSLRLSCIASGFTFDGTTMGWYRQAPGDECELVSDISSDGNTYYSDSVKGRFTISQGNAKNTVYLQMNSLTPEDTAVYYCAAAEFRLLLRPLCRQDDYGYWGQGTLVTVSSEPKIPQPQPKPQPQPQPQPKPQPKPEPECTCPKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 363 aa, synthetic construct
QVQLQESGGGLVQPGGSLRLSCAASGTTGDYKYLAWFRQGPGKEREGIAGLNTGGGSAYYADSVKGRFTISQDNVKRLLYLQLNNLKPEDTAMYYCAARRDYIHMTNIQALGLRPQWFSDWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 354 aa, synthetic construct
EVQLVESGGGSVQAQGSLRLSCTVSGSTYSSNFMAWFRQAPGKEREWVATITTGAGNTAYADSVKGRFTISEDNAKRTAYLQMNSLKPEDTAVYYCAAGRGGFWRPAEYDFWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 364 aa, synthetic construct
QVQLLESGGGSVQAGGSLRLSCAASGFTFSSGCMGWFRQAPGKGREGVATIYTRNDFTYYSDSVKGRFTISRDNSKNTVYLQMNSLRAEDTAVYYCAAGSPFRYAGAWNVCGLKEEDYNYWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - DNA, 1092 bp, synthetic construct
  source 1..1092: mol_type = other DNA
caggtgcagctgctggagagcggcggcggaagcgtgcaggctggaggatccctgcggctgagctgtgctgcctccggcttcaccttcagctccggctgcatgggctggttcaggcaggcccctggcaagggccgggagggagttgctaccatctacacccggaatgacttcacctattactccgacagcgtgaagggcaggttcaccatcagccgggacaactccaagaacaccgtgtatctgcagatgaatagcctgcgggccgaggacaccgccgtgtactattgcgctgccggcagccccttcaggtacgctggcgcttggaatgtgtgtggcctgaaggaggaggattacaattattggggccagggcaccctggtgaccgtgagcagcggagagccaaagtccagcgataagacccacacctgccccccttgccctgctcccgagctgctgggaggccctagcgtgttcctgttcccccctaagcctaaggacaccctgatgatcagccggacccccgaggtgacctgtgtggtggtggatgtgagccacgaggatcctgaggtgaagttcaactggtatgtggacggcgtggaggtgcacaacgccaagaccaagcctagggaggagcagtataactccacctatagggtggtgagcgtgctgaccgtgctgcaccaggattggctgaatggcaaggagtacaagtgcaaggtgagcaataaggctctgcctgctcctatcgagaagaccatctccaaggctaagggccagcccagggagcctcaggtgtacaccctgcctcccagcagggacgagctgaccaagaatcaggtgagcctgacctgcctggtgaagggcttctacccctccgatatcgctgtggagtgggagagcaacggccagcctgagaacaactacaagaccaccccccccgtgctggattccgatggctccttcttcctgtacagcaagctgaccgtggataagagccggtggcagcagggcaacgtgttcagctgctccgtgatgcacgaggccctgcacaaccactacacccagaagagcctgagcctgagccctggcaag

SEQ ID NO: 53 - protein, 364 aa, synthetic construct
QVQLLESGGGSVQAGGSLRLSCAASGHTYSSGCMGWFRQAPGKGREGVATIYTRNDFTYYSDSVKGRFTISRDNSKNTVYLQMNSLRAEDTAVYYCAAGSPFRYAGAWNVCGLKEEDYNYWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 1092 bp, synthetic construct
  source 1..1092: mol_type = other DNA
caggtgcagctgctggagtccggcggcggatccgtgcaggctggaggatccctgcggctgagctgcgctgcttccggccacacctactcctccggctgcatgggctggttcaggcaggctcctggcaagggccgggagggagtggctaccatctataccaggaacgacttcacctactattccgattccgtgaagggcaggttcaccatcagcagggataactccaagaacaccgtgtatctgcagatgaatagcctgcgggccgaggataccgccgtgtactactgcgccgccggcagccccttccggtacgctggagcttggaacgtgtgcggcctgaaggaggaggactacaactattggggccagggcaccctggtgaccgtgagctccggagagccaaagtccagcgataagacccacacctgccccccttgccctgctcccgagctgctgggaggccctagcgtgttcctgttcccccctaagcctaaggacaccctgatgatcagccggacccccgaggtgacctgtgtggtggtggatgtgagccacgaggatcctgaggtgaagttcaactggtatgtggacggcgtggaggtgcacaacgccaagaccaagcctagggaggagcagtataactccacctatagggtggtgagcgtgctgaccgtgctgcaccaggattggctgaatggcaaggagtacaagtgcaaggtgagcaataaggctctgcctgctcctatcgagaagaccatctccaaggctaagggccagcccagggagcctcaggtgtacaccctgcctcccagcagggacgagctgaccaagaatcaggtgagcctgacctgcctggtgaagggcttctacccctccgatatcgctgtggagtgggagagcaacggccagcctgagaacaactacaagaccaccccccccgtgctggattccgatggctccttcttcctgtacagcaagctgaccgtggataagagccggtggcagcagggcaacgtgttcagctgctccgtgatgcacgaggccctgcacaaccactacacccagaagagcctgagcctgagccctggcaag

SEQ ID NO: 55 - protein, 364 aa, synthetic construct
QVQLLESGGGSVQAGGSLRLSCAASGHTYSSGCMGWFRQAPGKGREGVATIYTRNDFTYYSDTVKGRFTISRDNSKNTVYLQMNSLRAEDTAVYYCAAGSPFRYAGAWNVCGLKEEDYNYWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 1092 bp, synthetic construct
  source 1..1092: mol_type = other DNA
caggtgcagctgctggagagcggcggcggaagcgtgcaggctggaggaagcctgaggctgagctgcgccgccagcggacacacctacagctccggctgtatgggctggttcaggcaggcccccggcaagggacgggagggagttgctaccatctacacccggaatgacttcacctactattccgataccgtgaagggcaggttcaccatcagcagggacaactccaagaataccgtgtacctgcagatgaactccctgcgggccgaggacaccgccgtgtattactgtgctgctggctcccccttcaggtatgccggcgcttggaatgtgtgcggcctgaaggaggaggactataattattggggccagggcaccctggtgaccgtgagcagcggagagccaaagtccagcgataagacccacacctgccccccttgccctgctcccgagctgctgggaggccctagcgtgttcctgttcccccctaagcctaaggacaccctgatgatcagccggacccccgaggtgacctgtgtggtggtggatgtgagccacgaggatcctgaggtgaagttcaactggtatgtggacggcgtggaggtgcacaacgccaagaccaagcctagggaggagcagtataactccacctatagggtggtgagcgtgctgaccgtgctgcaccaggattggctgaatggcaaggagtacaagtgcaaggtgagcaataaggctctgcctgctcctatcgagaagaccatctccaaggctaagggccagcccagggagcctcaggtgtacaccctgcctcccagcagggacgagctgaccaagaatcaggtgagcctgacctgcctggtgaagggcttctacccctccgatatcgctgtggagtgggagagcaacggccagcctgagaacaactacaagaccaccccccccgtgctggattccgatggctccttcttcctgtacagcaagctgaccgtggataagagccggtggcagcagggcaacgtgttcagctgctccgtgatgcacgaggccctgcacaaccactacacccagaagagcctgagcctgagccctggcaag

SEQ ID NO: 57 - protein, 361 aa, synthetic construct
QVQLVESGGGSVQPGGSLRLSCAASGYSYSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 1083 bp, synthetic construct
  source 1..1083: mol_type = other DNA
caggtgcagctggtggagtccggcggcggaagcgtgcagcctggaggaagcctgaggctgtcctgcgctgcctccggctatagctacagcgctaactgcgtgggctggttccggcaggctcctggcaagggccgggagggagtgtccgctatcaacaccggcggcgagaccgctacctacgccgacttcgtgaagggccggttcaccatctcccgggataacgctaagaataccgtgtacctgcagatgaatagcctgcgggctgaggataccgccgtgtattattgtgctgccgtgggcacccggaagtactgggacgagtgcagcctgagccagcacgcctacaagaattggggccagggcaccctggtgaccgtgtccagcggagagccaaagtccagcgataagacccacacctgccccccttgccctgctcccgagctgctgggaggccctagcgtgttcctgttcccccctaagcctaaggacaccctgatgatcagccggacccccgaggtgacctgtgtggtggtggatgtgagccacgaggatcctgaggtgaagttcaactggtatgtggacggcgtggaggtgcacaacgccaagaccaagcctagggaggagcagtataactccacctatagggtggtgagcgtgctgaccgtgctgcaccaggattggctgaatggcaaggagtacaagtgcaaggtgagcaataaggctctgcctgctcctatcgagaagaccatctccaaggctaagggccagcccagggagcctcaggtgtacaccctgcctcccagcagggacgagctgaccaagaatcaggtgagcctgacctgcctggtgaagggcttctacccctccgatatcgctgtggagtgggagagcaacggccagcctgagaacaactacaagaccaccccccccgtgctggattccgatggctccttcttcctgtacagcaagctgaccgtggataagagccggtggcagcagggcaacgtgttcagctgctccgtgatgcacgaggccctgcacaaccactacacccagaagagcctgagcctgagccctggcaag

SEQ ID NO: 59 - protein, 361 aa, synthetic construct
QVQLVESGGGSVQPGGSLRLSCAASGFTFSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 1083 bp, synthetic construct
  source 1..1083: mol_type = other DNA
caggtgcagctggtggagtccggcggcggatccgtgcagcctggaggatccctgaggctgagctgcgccgccagcggattcaccttcagcgccaactgcgtgggctggttccggcaggctcctggcaagggccgggagggagtgagcgctatcaacaccggcggcgagaccgccacctatgccgacttcgtgaagggccggttcaccatctccagggataatgctaagaacaccgtgtacctgcagatgaatagcctgagggccgaggataccgccgtgtattactgtgccgccgtgggcaccaggaagtattgggacgagtgctccctgtcccagcacgcttacaagaactggggccagggcaccctggtgaccgtgagcagcggagagccaaagtccagcgataagacccacacctgccccccttgccctgctcccgagctgctgggaggccctagcgtgttcctgttcccccctaagcctaaggacaccctgatgatcagccggacccccgaggtgacctgtgtggtggtggatgtgagccacgaggatcctgaggtgaagttcaactggtatgtggacggcgtggaggtgcacaacgccaagaccaagcctagggaggagcagtataactccacctatagggtggtgagcgtgctgaccgtgctgcaccaggattggctgaatggcaaggagtacaagtgcaaggtgagcaataaggctctgcctgctcctatcgagaagaccatctccaaggctaagggccagcccagggagcctcaggtgtacaccctgcctcccagcagggacgagctgaccaagaatcaggtgagcctgacctgcctggtgaagggcttctacccctccgatatcgctgtggagtgggagagcaacggccagcctgagaacaactacaagaccaccccccccgtgctggattccgatggctccttcttcctgtacagcaagctgaccgtggataagagccggtggcagcagggcaacgtgttcagctgctccgtgatgcacgaggccctgcacaaccactacacccagaagagcctgagcctgagccctggcaag

SEQ ID NO: 61 - protein, 463 aa, synthetic construct
QRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLR

SEQ ID NO: 62 - protein, 234 aa, synthetic construct
VVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 63 - protein, 5 aa, synthetic construct
TYGMH

SEQ ID NO: 64 - protein, 17 aa, synthetic construct
VIWDDGSYKYYGDSVKG

SEQ ID NO: 65 - protein, 16 aa, synthetic construct
DGITMVRGVMKDYFDY

SEQ ID NO: 66 - protein, 11 aa, synthetic construct
RASQDISSALV

SEQ ID NO: 67 - protein, 7 aa, synthetic construct
DASSLES

SEQ ID NO: 68 - protein, 9 aa, synthetic construct
QQFNSYPLT

SEQ ID NO: 69 - protein, 125 aa, synthetic construct [sequence omitted]

SEQ ID NO: 70 - protein, 107 aa, synthetic construct [sequence omitted]

SEQ ID NO: 71 - protein, 502 aa, synthetic construct
QVQLVESGGDSVQAGGSLRLSCAASAHAYSSGCMGWFRQAPGKGREGVATIYTRNDFTYYSDSVKGRFTISRDDAKNTVYLQVNSLKAEDTAMYYCAAGSPFRYAGAWNVCGLKEEDYNYWGQGTLVTVSSGGGGSGGGGSQVQLVESGGGLVQPGGSLRLSCAASGYSYSANCVGWFYQAPGKEREGVAAINTGGETATYADFVKGRFTISQDYAKNTVYLQMNSLKSEDTGMYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 72 - DNA, 1506 bp, synthetic construct
  source 1..1506: mol_type = other DNA
caagtgcagctggtcgagtccggcggcgactccgtgcaggctggcggatctctgagactgtcttgcgctgcttctgcccacgcttactccagcggctgcatgggctggttcagacaggcccctggcaagggcagagaaggagtggctaccatctacaccagaaacgactttacctattactccgattctgtcaagggcaggttcaccatctcccgggacgacgccaagaacaccgtgtacctgcaggtgaacagcctgaaggccgaggataccgccatgtactactgcgccgctggctcccctttccggtatgccggcgcctggaacgtgtgcggcctgaaagaagaagactacaactactggggccagggaaccttggtgaccgtctcatccgggggcggcggtagcggtggcggaggctctcaagtgcagctggtggaatctggcggaggactggttcagcctggcggctctctccggctgtcctgcgccgcctctggctactcctactctgccaactgcgtgggctggttctaccaggcccccggcaaggagagagagggcgtggccgccatcaacaccggcggcgagacagcaacctacgccgacttcgtgaaaggccggtttacaatctctcaggattatgctaagaataccgtgtacctgcagatgaactccctgaagtccgaggacacaggcatgtactactgtgctgctgtgggcacccggaagtactgggacgagtgctctctgagccagcacgcctacaaaaattggggccaaggcaccctggtgaccgtgtccagtggcgaacccaagtcctccgacaagacccacacctgccccccctgtcctgctccagagctgctgggcggcccttctgtgtttctgttccctcctaagcccaaggacaccctgatgatctccagaacccctgaggtgacctgtgtggtggtggacgtgtctcacgaggatcctgaagtgaagttcaactggtacgtggacggcgtggaagtgcataacgccaagacaaagcctagagaggaacagtacaactctacctacagagtggtgtccgtgctgaccgtgctgcaccaggactggctgaacggcaaagagtacaagtgcaaggtgtccaacaaggctctgcctgcccctatcgagaagaccatcagcaaggccaagggccagccccgcgagcctcaggtctgcaccctgcctccatccagagaagagatgaccaagaaccaggtttccctgtcttgtgctgtgaagggcttctaccctagcgacatcgccgtggaatgggagtctaatggccagcctgagaacaactacaagaccacacctcctgtgctcgactccgatggctccttcttcctggtgtccaagctgacggtggataagtctagatggcagcagggcaacgtgttctcctgctccgtgatgcacgaggccctgcataaccggttcacccagaagagcctgtccctgtctccaggaaaa

SEQ ID NO: 73 - protein, 502 aa, synthetic construct
QVQLLESGGGSVQAGGSLRLSCAASGFTFSSGCMGWFRQAPGKGREGVATIYTRNDFTYYSDSVKGRFTISRDNSKNTVYLQMNSLRAEDTAVYYCAAGSPFRYAGAWNVCGLKEEDYNYWGQGTLVTVSSGGGGSGGGGSQVQLVESGGGSVQPGGSLRLSCAASGYSYSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 74 - DNA, 1506 bp, synthetic construct
  source 1..1506: mol_type = other DNA
caggtgcagctgctggaatctggcggcggctctgtgcaggctggtggatccctgagactgtcttgtgctgcctccggctttaccttcagcagtggctgcatgggctggtttagacaggcccccggcaaaggcagagaaggcgtggccaccatctacaccagaaacgacttcacctattattccgattccgtgaaaggcagattcaccatcagccgcgacaactccaaaaacaccgtgtacctgcagatgaactccctgagagccgaggataccgctgtgtactactgcgccgccggctctcctttccggtacgccggagcttggaacgtgtgtggcctgaaggaagaggactacaactactggggacaaggcaccctggtgaccgtgtctagcggcggaggtggctcgggcggaggcggctctcaagtgcagctggtggaatccggcggcggctccgtgcaacccggaggctccctgcggttgtcctgtgctgcctctggctactcctactccgccaactgcgtaggatggttcagacaggcccctggcaagggcagagagggcgtgtccgctatcaacaccggcggcgagacagccacctacgccgacttcgtgaagggccggttcaccatctctcgggacaacgccaagaacactgtctatctgcagatgaactccctgagagcagaagataccgccgtgtactactgtgctgctgtgggcacccggaagtactgggacgagtgctctctgtcccagcacgcctacaagaattggggccagggcacactagtcaccgtctcctctggcgagcctaagtcctctgataagacccacacctgtcctccttgccctgctcctgagctgctgggcggcccttccgtcttcctgttccctccaaagcccaaggacacactgatgatctccaggacacctgaagtgacctgcgtggtggtggacgtgtctcatgaggaccccgaggtgaagttcaactggtacgtggatggcgtggaagtgcacaatgccaagaccaagcctcgggaggaacagtacaactctacctacagagtggtgtccgtgctgaccgtcctgcaccaggactggctgaacggcaaagagtacaagtgcaaggtgtccaacaaggccctgccagctcctatcgagaagacaatcagcaaggccaagggccagcctagagagcctcaggtgtgcaccctgcctccttctcgggaagagatgaccaagaaccaggtgagcctgtcctgcgccgtgaaaggcttctacccctccgacatcgccgtcgagtgggagtctaatggacagcccgagaacaactacaagaccacccctcctgtgctggactccgacggctctttcttcctggtttctaagctgaccgtggacaagtccagatggcagcagggcaacgtgttctcctgctccgtgatgcacgaagctctgcacaaccggtttacccagaagtctctgtctctcagcccaggcaag

SEQ ID NO: 75 - protein, 502 aa, synthetic construct
QVQLLESGGGSVQAGGSLRLSCAASGHTYSSGCMGWFRQAPGKGREGVATIYTRNDFTYYSDSVKGRFTISRDNSKNTVYLQMNSLRAEDTAVYYCAAGSPFRYAGAWNVCGLKEEDYNYWGQGTLVTVSSGGGGSGGGGSQVQLVESGGGSVQPGGSLRLSCAASGYSYSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 76 - DNA, 1506 bp, synthetic construct
  source 1..1506: mol_type = other DNA
caggttcagctgttggaatctggcggcggatctgttcaggctggcggatctctgagactgtcttgtgctgcttccggccacacctactcctctggctgtatgggatggttcagacaggcccctggcaagggaagagaaggcgtggccaccatctacacccggaacgacttcacctactactccgactccgtgaagggcagattcaccatcagccgggacaactccaagaacaccgtgtacctgcagatgaactccctgagagccgaggacaccgccgtgtactattgtgccgctggcagcccctttagatacgctggcgcttggaacgtgtgcggcctgaaagaagaggactacaactactggggccagggcaccctggtcacagtttctagcggaggcggaggatcaggtggcggtggatctcaggtgcagttggttgaaagtggcggaggctctgtgcaaccaggcggaagcctcagattgtcttgcgccgcttctggctactcctacagcgccaattgtgtcggctggtttaggcaggctcccggaaaaggcagagaaggggtgtccgctatcaacaccggcggagagacagccacctacgccgatttcgtgaaaggccggtttaccatctctcgcgataacgctaagaacacagtctatctccaaatgaacagcctgcgcgctgaggataccgctgtgtattactgcgctgccgtgggcaccagaaagtactgggacgagtgctccctgagccagcacgcctataagaattggggacagggaaccctcgtgaccgtgtcctctggcgaacctaagtcctccgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgcgctgtgaagggcttctacccttccgatatcgccgtggaatgggagagcaatggccagccagagaacaactacaagacaacccctcctgtgctggactccgacggctcattcttcctggtgtccaagctgacagtggacaagtccagatggcagcagggcaacgtgttctcctgctccgtgatgcacgaggccctgcacaacaggttcacccagaagtccctgtctctgagcccaggcaaa

SEQ ID NO: 77 - protein, 502 aa, synthetic construct
QVQLLESGGGSVQAGGSLRLSCAASGHTYSSGCMGWFRQAPGKGREGVATIYTRNDFTYYSDTVKGRFTISRDNSKNTVYLQMNSLRAEDTAVYYCAAGSPFRYAGAWNVCGLKEEDYNYWGQGTLVTVSSGGGGSGGGGSQVQLVESGGGSVQPGGSLRLSCAASGYSYSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 78 - DNA, 1506 bp, synthetic construct
  source 1..1506: mol_type = other DNA
caggttcagctgctggaatctggcggaggctccgtgcaggccggcggaagcctgagactgtcctgcgccgcctctggccatacatattcttccggctgcatgggctggttccggcaggctcctggcaagggcagggagggcgtggccacaatctacaccagaaacgacttcacctattattctgataccgtcaaaggcagattcaccatctctcgggacaactccaagaacaccgtgtacctgcagatgaactccctgcgggctgaggacaccgccgtctactactgcgccgctggctctccttttagatacgctggcgcctggaacgtgtgtggcctgaaggaagaagactacaactactggggccaaggcaccctggtgaccgtgtcctccggcggcggaggctctggaggcggcggcagccaagtgcagctggtggaaagcggcggtggctccgtgcagccaggcggctctctgcggctgtcctgtgccgcttctggctactcctactccgccaactgcgtgggctggttcagacaggcccccggcaagggccgggaaggcgtgtctgccatcaacaccggcggcgagacagctacctacgccgacttcgtgaagggcagatttaccatctccagagataacgctaagaataccgtgtacctgcagatgaacagcctgcgggccgaggacaccgctgtgtactactgtgctgccgtgggcacccggaagtactgggacgagtgcagtctgagccagcacgcctacaagaattggggccagggaacactggtcaccgtgtccagtggagaacctaagtcgtctgacaagacccacacctgtcctccatgccccgctcccgagctgctgggcggcccttctgtgttcctgttccctcctaagcccaaggacacactgatgatcagcagaacccctgaagtgacctgcgtggtggtggacgtgtcccacgaggatcctgaggtgaagttcaactggtacgtggacggcgtggaagtgcacaacgccaagaccaagcctcgcgaggagcagtacaactccacctacagagtggtctctgtactgaccgtgctgcaccaggactggctgaacggaaaagagtacaagtgcaaggtgtccaacaaggctctgcctgcccctatcgagaagaccatctccaaggccaaaggacagcctagagagcctcaagtgtgcaccctgcctccatctcgggaagagatgactaagaaccaggtctccctgtcctgtgccgtcaagggcttctacccctccgacatcgccgtggagtgggagtccaacggccagcctgagaacaactacaaaaccacacctcctgtgctggacagcgatggctccttcttcctcgtgtccaagctgaccgttgataagtccagatggcagcagggcaacgtgttctcttgctccgtgatgcacgaggccctgcataatagatttacccagaagtctctgtcgctctctcccggcaaa

SEQ ID NO: 79 - protein, 488 aa, synthetic construct
EVQLVESGGGSVQAQGSLRLSCTVSGSTYSSNFMAWFRQAPGKEREWVATITTGAGNTAYADSVKGRFTISEDNAKRTAYLQMNSLKPEDTAVYYCAAGRGGFWRPAEYDFWGQGTQVTVSSGGGGSQVQLVESGGGSVQPGGSLRLSCAASGYSYSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 80 - DNA, 1464 bp, synthetic construct
  source 1..1464: mol_type = other DNA
gaagtgcagctggtcgagtctggcggcggctctgtgcaggctcagggctccctgcggctgtcttgtaccgtttccgggtccacctactcttccaacttcatggcctggttccggcaggcccctggaaaagagagagaatgggtggctaccatcaccacaggcgctggcaacaccgcttatgccgactccgtcaagggcagattcaccatctctgaggacaacgccaagcggaccgcctacctgcagatgaacagcctgaagcctgaagataccgccgtgtactactgtgctgccggcagaggcggcttttggcggcccgctgagtacgacttctggggccaaggcactcaagtgacagtgtcctctggaggaggaggctctcaggtgcagctggtggaatctggcggcggctccgtgcaaccaggcggatctctgagactgtcctgcgctgcttccggctactcctactccgccaactgcgtgggctggttcagacaggcccctggcaaaggccgcgagggcgtctctgccatcaacaccggcggagagacagctacctacgccgattttgttaagggccggttcactatctccagagacaatgctaagaacaccgtgtacctgcagatgaactctctgagagccgaggacaccgctgtgtactactgcgccgctgtgggcacccggaagtactgggacgagtgctctctgtcccagcacgcctataagaattggggccagggcaccctcgtgaccgtgtcttcaggcgagcctaaaagcagcgataagacccacacctgccctccttgccccgctcctgagctgctgggcggaccttctgtctttctgttcccacctaagcccaaggacaccctgatgatcagccggacacctgaggtgacctgtgtggtggtggacgtgtctcatgaggatcctgaggtgaagttcaactggtacgtggatggcgtggaagtgcacaatgccaagaccaagcctagagaagagcagtacaactccacatacagagtggtctcggtgctgaccgtgctgcaccaggactggctgaacggcaaagaatacaagtgcaaggtgtccaacaaggctctgcctgcccccatcgagaagacaatctccaaggccaagggccagcctcgggaacctcaggtgtgcaccctgcccccctccagggaagaaatgacaaagaaccaggtgtccctatcctgtgccgtgaaaggcttctacccttccgacatcgccgtggagtgggagtccaacggccagcccgagaacaactacaaaaccacccctccagtgctggactctgacggctccttcttcctggtgagcaagctgaccgtggacaagtctagatggcaacagggaaacgtgttcagctgctccgtcatgcacgaggccctgcacaacagatacacccagaagtccctgagcctgtctcctggcaag

SEQ ID NO: 81 - protein, 488 aa, synthetic construct
EVQLVESGGGSVQAGGSLRLSCAVSGFTVSSNFMAWFRQAPGKGREWVSTITTGAGNTAYADSVKGRFTISEDNSKNTLYLQMNSLRAEDTAVYYCAAGRGGFWRPAEYDFWGQGTTVTVSSGGGGSQVQLVESGGGSVQPGGSLRLSCAASGYSYSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 82 - DNA, 1464 bp, synthetic construct
  source 1..1464: mol_type = other DNA
gaggtgcagctggtggaatcaggcggaggctccgtgcaggccggcggctctttgagactgtcttgtgccgtgtccggcttcaccgtgtcttctaacttcatggcctggttcagacaggctcctggcaagggcagagagtgggtcagcaccatcaccaccggcgctggaaacaccgcctacgccgactccgtgaaagggcgctttacaatcagcgaggacaactccaagaataccctgtacctgcagatgaactctctgcgggccgaggataccgccgtgtactactgtgctgctggcagaggcggcttttggcggccagctgagtacgacttctggggccaaggcaccacagtcaccgtgtcctctggcggaggtggcagccaagtgcagctggtcgaatctggaggtggctccgttcagcctggcggctctctgagactgtcttgcgccgcttccggctactcttactccgccaactgcgtgggctggttccggcaggcccccggcaaaggcagagaaggcgtgagcgccatcaacaccggcggcgaaaccgctacctatgccgatttcgtgaagggaagattcaccatctctcgggacaacgccaagaacaccgtgtatctgcagatgaactctctgagagccgaggatacagctgtgtactactgcgctgctgtgggcaccagaaagtactgggacgagtgctccctgtcccagcacgcctataagaactggggacagggcacactcgtgaccgtgtctagcggcgagcctaaaagctctgacaaaacccacacctgtcctccttgccccgctcctgagctgctgggcggcccttccgtgtttctgttccctcccaagcccaaggacacactgatgatctccagaaccccggaagtgacctgcgtggtggtggacgtgtctcacgaggaccctgaggtcaagttcaactggtacgtggatggcgtggaagtgcataatgctaagaccaagcctcgggaggaacagtacaactccacctacagagtggtttccgtgctgaccgtgctgcaccaagactggctgaacggcaaagagtacaagtgcaaggtgtccaacaaggctctgcctgcccccatcgagaagacgatctccaaggccaagggccagcctagggaacctcaggtgtgcaccctgcctccttcccgggaagagatgaccaaaaaccaggtcagcctgtcttgcgccgtgaagggcttctacccctccgacatcgccgtggaatgggagtccaacggccagcccgagaataactacaagaccacccctcctgtgctcgatagcgacggctccttcttcctggtgtctaagctgacagtggacaagtctagatggcagcagggcaacgtgttctcctgttccgtgatgcacgaggccctgcacaaccggtacacccagaagtccctgagcctgtctccaggcaag

SEQ ID NO: 83 - protein, 488 aa, synthetic construct
EVQLVESGGGSVQAGGSLRLSCAVSGFTVSSNFMAWFRQAPGKGREWVSTITTGAGNTAYADSVKGRFTISEDNSKNTAYLQMNSLRAEDTAVYYCAAGRGGFWRPAEYDFWGQGTTVTVSSGGGGSQVQLVESGGGSVQPGGSLRLSCAASGYSYSANCVGWFRQAPGKGREGVSAINTGGETATYADFVKGRFTISRDNAKNTVYLQMNSLRAEDTAVYYCAAVGTRKYWDECSLSQHAYKNWGQGTLVTVSSGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 84 - DNA, 1464 bp, synthetic construct
  source 1..1464: mol_type = other DNA
gaagtgcagctggtggaatccggcggcggatctgtgcaggccgggggctccctgagactgtcttgcgccgtgtccggcttcaccgtgtcttctaacttcatggcctggtttcgccaggcacctggcaagggcagagagtgggtgtctaccatcaccaccggcgctgggaacaccgcttacgccgattctgtgaaaggccggttcaccatctccgaggataattccaagaacacagcttatctgcagatgaacagcctgagagccgaggacaccgccgtatactactgtgctgctggcagaggcggcttttggcggcctgctgagtacgacttctggggccagggcaccacagtcacagtttcctctggaggcggtggctctcaagtgcagctggtggagtctgggggaggctctgtccaacctggaggatctctgcggctgagctgcgccgcctctggctactcctacagcgccaactgcgtgggctggttcagacaggcccctggaaagggcagagaaggcgtttctgccatcaacaccggcggcgagacagccacatacgccgacttcgtgaagggcagattcaccatcagtcgggacaacgccaaaaacaccgtgtacctgcagatgaactccctccgggccgaggacaccgctgtgtactactgcgctgctgtcggcaccagaaagtactgggacgagtgctctctgagccagcacgcctacaaaaattggggccaaggcaccctcgtgacagtgagcagcggcgaacccaagtccagcgacaagacccacacctgccctccttgtcctgccccagagctgctgggcggcccttccgtgtttctgttcccacccaagcctaaggacactctgatgatcagcagaacccctgaggtgacctgtgtggtggtggacgtgtctcacgaagatcctgaagtcaagttcaactggtacgtggacggcgtggaagtgcacaacgctaagaccaagcccagagaagagcagtacaacagcacctacagagtggtctccgtgctgaccgtgctgcaccaggactggctgaacggcaaagagtataagtgcaaggtgtccaacaaggctctgcccgctcctatcgagaagaccatctccaaggccaagggccagcctcgggagcctcaggtgtgcaccctgcctccctcccgggaagagatgaccaagaaccaggtgtccttgtcttgtgctgtgaagggcttctacccttcggacatcgccgtggaatgggagtccaatggacagcccgagaacaactacaagacaacccctcctgtgctggactccgatggctccttcttcctggtgtccaagctgaccgtggataagtctaggtggcagcagggcaacgtgttctcctgctccgtgatgcacgaggccctgcataacagatacacccagaagtccctgtccctgtctcctggcaaa

SEQ ID NO: 85 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - DNA, 1365 bp, synthetic construct
  source 1..1365: mol_type = other DNA
caggtgcagctggtggagtccggcggcggagtggtgcagccaggaagatccctgcggctgagctgtgccgcttccggcttcaccttctccacctacggcatgcactgggtgcggcaggcccccggaaagggactggagtgggtggctgtgatctgggacgacggctcctacaagtattacggcgattccgtgaagggccggttcaccatctcccgggacaactccaagaacaccctgtacctgcagatgaatagcctgagggccgaggacaccgctgtgtactactgtgcccgggacggcatcaccatggtgcggggagtgatgaaggattacttcgactactggggccagggcaccctggtgaccgtgagctccgccagtaccaaaggcccttccgtgtttcctctggccccatcctccaagagtacatctggaggcaccgctgctctgggctgcctggtgaaggactacttccccgagcccgtgaccgtcagctggaactccggcgccctgacctctggcgttcatacctttcccgccgttctgcagagctctggcctgtacagcctgtcttccgtcgtgaccgtgccttctagctctctgggaacacagacctacatctgcaacgtgaaccacaagcccagcaacaccaaggtggacaagcgggtggaacccaagtcctgcgacaaaacccacacctgtcctccttgtccagcccctgagctgctgggcggcccttctgtctttctgttccctcctaagcctaaggacacgctcatgatctcccggacacctgaagtgacctgcgtggtggtggatgtgtctcacgaggaccctgaggtgaagttcaactggtacgtggacggcgtggaagtccataacgccaagaccaagcctagagaggagcagtacaattccacctatagggtggtgagcgtgctgaccgtgctgcaccaggattggctgaatggcaaggagtacaagtgcaaggtgagcaataaggctctgcctgctcccatcgagaagaccatctccaaggctaagggccagcctagggagccccaggtgtataccctgcctccctgccgggaggagatgaccaagaaccaggtgtccctgtggtgcctggtgaagggcttctacccttccgacatcgctgtggagtgggagtccaatggccagcccgagaataattacaagaccacccctcccgtgctggattccgacggctccttcttcctgtactccaagctgaccgtggacaagagcaggtggcagcagggcaacgtgttcagctgtagcgtgatgcacgaggccctgcacaaccactacacccagaagtccctgagcctgagccccggcaag

SEQ ID NO: 87 - protein, 214 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - DNA, 642 bp, synthetic construct
  source 1..642: mol_type = other DNA
gctatccagctgacccagtcccctagctccctgagcgcctccgtgggcgacagggtgaccatcacctgcagggccagccaggatatctcctccgccctggtgtggtaccagcagaagcccggcaaggcccccaagctgctgatctatgacgctagctccctggagtccggcgtgccctccaggttcagcggctccgagtccggcaccgacttcaccctgaccatctccagcctgcagcccgaggacttcgccacctattattgtcagcagttcaatagctaccccctgaccttcggcggcggcaccaaggtggagatcaagaggaccgtggccgcccctagcgtgttcatcttccctcctagcgacgagcagctgaagagcggcaccgcctccgtggtgtgcctgctgaacaacttctatcccagggaggctaaggtgcagtggaaggtggataacgccctgcagtccggcaatagccaggagagcgtgaccgagcaggattccaaggattccacctactccctgagcagcaccctgaccctgagcaaggctgattacgagaagcacaaggtgtatgcttgtgaggtgacccaccagggcctgagcagccccgtgaccaagtccttcaacaggggcgagtgt

SEQ ID NO: 89 - protein, 16 aa, synthetic construct
GEPKSSDKTHTCPPCP

SEQ ID NO: 90 - protein, 15 aa, synthetic construct
EPKSCDKTHTCPPCP

SEQ ID NO: 91 - protein, 5 aa, synthetic construct
GGGGS

SEQ ID NO: 92 - protein, 330 aa, synthetic construct [sequence omitted]

(19) United States
(12) Patent Application Publication
(10) Pub. No.: US 2026/0335457 A1
(43) Pub. Date: Oct. 29, 2026

(54) ANTIBODY-DRUG CONJUGATE TARGETING EGFR AND C-MET

(71) Applicant: Chia Tai Tianqing Pharmaceutical Group Co., Ltd., Lianyungang (CN)

(72) Inventors: Zhengping ZHANG, Lianyungang (CN); Yimin MA, Lianyungang (CN); Yaping ZHOU, Lianyungang (CN); Nianqin YU, Lianyungang (CN); Hongmei CHEN, Lianyungang (CN); Chen SHEN, Lianyungang (CN); Bing ZHANG, Lianyungang (CN); Tongjie XU, Lianyungang (CN)

(21) Appl. No.: 19/532,354

(22) Filed: Feb. 6, 2026

Related U.S. Application Data
(63) Continuation of application No. PCT/CN2025/129404, filed on Oct. 22, 2025.

(30) Foreign Application Priority Data
Oct. 23, 2024 (CN) 202411488108.X

(51) Int. Cl. A61K 47/68; A61P 35/00; C07K 16/28

(52) U.S. Cl. CPC A61K 47/68037; A61K 47/6879; A61K 47/6889; A61P 35/00; C07K 16/2863

(57) ABSTRACT

The disclosure pertains to the field of biological therapies. An antibody-drug conjugate targeting EGFR and c-Met is provided, wherein the antibody-drug conjugate includes a multispecific antibody, a linker, and a cytotoxic drug that are linked. Further provided are a pharmaceutical composition including the antibody-drug conjugate and use thereof. The provided antibody-drug conjugate achieves excellent anti-tumor activity or/and good safety. The provided antibody-drug conjugate can be used in the treatment of tumors.

### CROSS-REFERENCE TO RELATED APPLICATIONS

This application is a continuation of International Application No. PCT/CN2025/129404, filed Oct. 22, 2025, which claims priority under 35 U.S.C. § 119 (b) to Chinese Patent Application No. CN 202411488108.X, filed on Oct. 23, 2024, the disclosures of which are incorporated herein by reference in their entireties.

### REFERENCE TO SEQUENCE LISTING SUBMITTED ELECTRONICALLY

This application contains a sequence listing, which is submitted electronically as a XML formatted sequence listing with a file name “Substitute_Sequence_Listing”, creation date of Jul. 2, 2026, and having a size of 123,361 bytes. The sequence listing submitted electronically is part of the specification and is herein incorporated by reference in its entirety.

### TECHNICAL FIELD

The present disclosure pertains to the field of biological therapies, and relates to an antibody-drug conjugate targeting EGFR and c-Met. The antibody-drug conjugate comprises a multispecific antibody, a linker, and a cytotoxic drug that are linked. The present disclosure further relates to use of the antibody and the antibody-drug conjugate in preparing a medicament for treating a tumor.

### BACKGROUND

Epidermal growth factor receptor (EGFR, also known as ErbB1 or HER1) is a 170 kDa type I transmembrane glycoprotein encoded by the proto-oncogene c-erbB1, and is one of the receptor tyrosine kinases (RTKs). EGFR is a member of the human epidermal growth factor receptor (HER) family, which includes HER2 (ErbB2), HER3 (ErbB3), and HER4 (ErbB4). Increased expression or kinase activity of EGFR is associated with a series of human tumors, making EGFR an attractive target for tumor therapy.

The c-mesenchymal-epithelial transition factor (c-Met) is one of the receptor tyrosine kinases and a heterodimer of approximately 190 kDa formed by linking a 50 kDa extracellular chain (a chain) and a 145 kDa transmembrane chain (β chain). The transmembrane chain (β chain) includes a SEMA domain (sema homology region, SEMA), a PSI domain (plexin semaphorin-integrin, PSI), 4 immunoglobulin-like repeat domains (immunoglobulin-like regions in plexins and transcription factors, IPT), a transmembrane domain, a juxtamembrane domain (JM), a tyrosine kinase domain (TK), and a carboxy-terminal tail region (carboxyl terminal, CT). c-Met is a receptor expressed on the cell surface, in which the SEMA domain is one of the important elements for ligand binding and is considered as a binding site of its ligand, hepatocyte growth factor (HGF). HGF is synthesized by mesenchymal cells, fibroblasts, and smooth muscle cells, and activates the HGF/c-Met signaling through a paracrine mechanism to exert its biological functions.

In various tumors, due to the overexpression of c-Met and HGF and the paracrine and autocrine positive feedback loops formed by HGF and c-Met, the HGF/c-Met signaling pathway is abnormally activated, and the growth, invasion, migration, and angiogenesis of tumor cells are promoted. The abnormal expression of the c-Met gene is present in many cancer types, such as brain cancer, breast cancer, colorectal cancer, gastric cancer, head and neck cancer, lung cancer, and liver cancer. Immunotherapeutic agents such as antibodies that bind to c-Met are able to block the binding of HGF to c-Met.

In addition to conventional IgG1 of a four-chain structure, IgG antibodies in camelids also include IgG2 and IgG3 heavy-chain only antibodies (HcAbs) free of light chains. The single-variable-region domains (V_{H}Hs) of heavy-chain only antibodies are capable of specifically binding to antigens and have relatively high affinity for antigens. Based on their uniqueness, V_{H}H domains used as part of an antibody or an antigen-binding fragment have more significant advantages over conventional antibody fragments (such as scFvs and Fabs): for example, only a single domain is required to specifically bind to an antigen with high affinity; they can be easily transformed into polyvalent and multispecific formats; V_{H}H domains are highly soluble and do not tend to aggregate; the V_{H}H molecules are small and thus have relatively high tissue permeability; V_{H}Hs do not need to be paired with light chains, and thus there is no light and heavy chain mismatch problem about forming bispecific or multispecific antibodies.

Antibody-drug conjugates (ADCs) are a class of drugs that combine the high specificity of therapeutic antibodies and the high killing activity of cytotoxic drugs. The therapeutic antibody moiety is linked to the cytotoxic drug moiety via an intermediate linker moiety. About 60% of all tumor patients resistant to EGFR tyrosine kinase inhibitors have increased c-Met expression, c-Met amplification, or increased HGF, indicating that c-Met will be activated as a compensatory pathway in the case of EGFR inhibition. While antibody-drug conjugates targeting EGFR and c-Met targets have been reported in the literature for anti-tumor applications, existing research remains limited, underscoring the need for additional available options.

### BRIEF SUMMARY

In one aspect, the present disclosure provides an antibody-drug conjugate, a stereoisomer thereof, a pharmaceutically acceptable salt thereof, or a solvate of the antibody-drug conjugate, the stereoisomer thereof, or the pharmaceutically acceptable salt thereof, comprising a multispecific antibody targeting EGFR and c-Met, wherein the multispecific antibody comprises
(i) a first antigen-binding portion binding to a first antigen;
(ii) a second antigen-binding portion binding to a second antigen; and
(iii) a third antigen-binding portion binding to the first antigen,
wherein the first antigen is c-Met, and the second antigen is EGFR; and the first antigen-binding portion and the third antigen-binding portion are both single variable domains and each independently comprise any one of the following:
(1) a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
(2) a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, wherein X₁ is S or T;
(3) a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9;
(4) a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12;
(5) a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15;
(6) a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18;
(7) a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21; or
(8) a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24.

In some embodiments, the first antigen-binding portion and the third antigen-binding portion each independently comprise any one of the following:
(2) a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, wherein X₁ is S or T;
(3) a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; or
(8) a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24.

In some embodiments, the first antigen-binding portion and the third antigen-binding portion each independently comprise any one of the following:
(2) a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, wherein X₁ is S or T; or
(3) a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9.

In some embodiments, the first antigen-binding portion and the third antigen-binding portion each independently comprise any one of the following:
(3) a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; or
(8) a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24.

In some embodiments, the first antigen-binding portion and the third antigen-binding portion each independently comprise a CDR1, a CDR2, and a CDR3 of a single variable domain having the amino acid sequence set forth in SEQ ID NO: 25, 26, 27, 28, 29, 30, 31, 32, 33, 34, 35, 36, 37, 38, or 39. In some embodiments, the first antigen-binding portion and the third antigen-binding portion each independently comprise a CDR1, a CDR2, and a CDR3 of a single variable domain having the amino acid sequence set forth in SEQ ID NO: 35, 36, or 38. The CDRs are defined, for example, according to Kabat, IMGT, Chothia, Contact, AbM, and/or CCG.

In some embodiments, the first antigen-binding portion and the third antigen-binding portion each independently comprise a CDR1, a CDR2, and a CDR3 of a single variable domain having the amino acid sequence set forth in SEQ ID NO: 35 or 36. In some other embodiments, the first antigen-binding portion and the third antigen-binding portion each independently comprise a CDR1, a CDR2, and a CDR3 of a single variable domain having the amino acid sequence set forth in SEQ ID NO: 36 or 38. The CDRs are defined, for example, according to Kabat, IMGT, Chothia, Contact, AbM, and/or CCG.

In some specific embodiments, the first antigen-binding portion comprises a CDR1, a CDR2, and a CDR3 of a single variable domain having the amino acid sequence set forth in SEQ ID NO: 36, and the third antigen-binding portion comprises a CDR1, a CDR2, and a CDR3 of a single variable domain having the amino acid sequence set forth in SEQ ID NO: 35. In some other specific embodiments, the first antigen-binding portion comprises a CDR1, a CDR2, and a CDR3 of a single variable domain having the amino acid sequence set forth in SEQ ID NO: 36, and the third antigen-binding portion comprises a CDR1, a CDR2, and a CDR3 of a single variable domain having the amino acid sequence set forth in SEQ ID NO: 38. The CDRs are defined, for example, according to Kabat, IMGT, Chothia, Contact, AbM, and/or CCG.

In some embodiments, the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; and the third antigen-binding portion comprises: a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, wherein X₁ is S or T. Further, in some embodiments, the first antigen-binding portion comprises the amino acid sequence set forth in SEQ ID NO: 41, and the third antigen-binding portion comprises the amino acid sequence set forth in SEQ ID NO: 40. In some embodiments, the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 36, and the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 35. In some specific embodiments, the first antigen-binding portion comprises the amino acid sequence set forth in SEQ ID NO: 36, and the third antigen-binding portion comprises the amino acid sequence set forth in SEQ ID NO: 35.

In some other embodiments, the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; and the third antigen-binding portion comprises: a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24. Further, in some embodiments, the first antigen-binding portion comprises the amino acid sequence set forth in SEQ ID NO: 41, and the third antigen-binding portion comprises the amino acid sequence set forth in SEQ ID NO: 42. In some embodiments, the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 36, and the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 38. In some specific embodiments, the first antigen-binding portion comprises the amino acid sequence set forth in SEQ ID NO: 36, and the third antigen-binding portion comprises the amino acid sequence set forth in SEQ ID NO: 38. In some embodiments, the first antigen-binding portion and the third antigen-binding portion bind to different epitopes of c-Met.

The second antigen-binding portion provides the ability to bind to EGFR. In some embodiments, the second antigen-binding portion comprises a heavy chain variable region and a light chain variable region; the heavy chain variable region comprises: an HCDR1 comprising the amino acid sequence set forth in SEQ ID NO: 63, an HCDR2 comprising the amino acid sequence set forth in SEQ ID NO: 64, and an HCDR3 comprising the amino acid sequence set forth in SEQ ID NO: 65; the light chain variable region comprises: an LCDR1 comprising the amino acid sequence set forth in SEQ ID NO: 66, an LCDR2 comprising the amino acid sequence set forth in SEQ ID NO: 67, and an LCDR3 comprising the amino acid sequence set forth in SEQ ID NO: 68.

Further, in some embodiments, the second antigen-binding portion comprises a heavy chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 69, and a light chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 70. In some specific embodiments, the second antigen-binding portion comprises a heavy chain variable region and a light chain variable region; the heavy chain variable region comprises the amino acid sequence set forth in SEQ ID NO: 69, and the light chain variable region comprises the amino acid sequence set forth in SEQ ID NO: 70.

In some embodiments, the multispecific antibody further comprises an Fc domain composed of two Fc polypeptides.

In some specific embodiments, in the multispecific antibody, the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; the third antigen-binding portion comprises: a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; and the second antigen-binding portion comprises a heavy chain variable region and a light chain variable region, the heavy chain variable region comprises: an HCDR1 comprising the amino acid sequence set forth in SEQ ID NO: 63, an HCDR2 comprising the amino acid sequence set forth in SEQ ID NO: 64, and an HCDR3 comprising the amino acid sequence set forth in SEQ ID NO: 65, and the light chain variable region comprises: an LCDR1 comprising the amino acid sequence set forth in SEQ ID NO: 66, an LCDR2 comprising the amino acid sequence set forth in SEQ ID NO: 67, and an LCDR3 comprising the amino acid sequence set forth in SEQ ID NO: 68, wherein X₁ is S or T. In further specific embodiments, the first antigen-binding portion comprises the amino acid sequence set forth in SEQ ID NO: 41, the third antigen-binding portion comprises the amino acid sequence set forth in SEQ ID NO: 40, the heavy chain variable region of the second antigen-binding portion comprises the amino acid sequence set forth in SEQ ID NO: 69, and the light chain variable region thereof comprises the amino acid sequence set forth in SEQ ID NO: 70. In further specific embodiments, the first antigen-binding portion comprises the amino acid sequence set forth in SEQ ID NO: 36, the third antigen-binding portion comprises the amino acid sequence set forth in SEQ ID NO: 35, the heavy chain variable region of the second antigen-binding portion comprises the amino acid sequence set forth in SEQ ID NO: 69, and the light chain variable region thereof comprises the amino acid sequence set forth in SEQ ID NO: 70.

In some other specific embodiments, in the multispecific antibody, the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; the third antigen-binding portion comprises: a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24; and the second antigen-binding portion comprises a heavy chain variable region and a light chain variable region, the heavy chain variable region comprises: an HCDR1 comprising the amino acid sequence set forth in SEQ ID NO: 63, an HCDR2 comprising the amino acid sequence set forth in SEQ ID NO: 64, and an HCDR3 comprising the amino acid sequence set forth in SEQ ID NO: 65, and the light chain variable region comprises: an LCDR1 comprising the amino acid sequence set forth in SEQ ID NO: 66, an LCDR2 comprising the amino acid sequence set forth in SEQ ID NO: 67, and an LCDR3 comprising the amino acid sequence set forth in SEQ ID NO: 68. In further specific embodiments, the first antigen-binding portion comprises the amino acid sequence set forth in SEQ ID NO: 41, the third antigen-binding portion comprises the amino acid sequence set forth in SEQ ID NO: 42, the heavy chain variable region of the second antigen-binding portion comprises the amino acid sequence set forth in SEQ ID NO: 69, and the light chain variable region thereof comprises the amino acid sequence set forth in SEQ ID NO: 70. In further specific embodiments, the first antigen-binding portion comprises the amino acid sequence set forth in SEQ ID NO: 36, the third antigen-binding portion comprises the amino acid sequence set forth in SEQ ID NO: 38, the heavy chain variable region of the second antigen-binding portion comprises the amino acid sequence set forth in SEQ ID NO: 69, and the light chain variable region thereof comprises the amino acid sequence set forth in SEQ ID NO: 70.

In the embodiments, in one optional configuration, the first antigen-binding portion and the third antigen-binding portion are both single variable domains, and the second antigen-binding portion is a Fab; and the first antigen-binding portion, at the C-terminus, is fused to the N-terminus of one of the Fc polypeptides of the Fc domain, the second antigen-binding portion, at the C-terminus of the Fab heavy chain or the Fab light chain, is fused to the N-terminus of the other Fc polypeptide of the Fc domain, and the third antigen-binding portion, at the C-terminus, is fused to the N-terminus of the first antigen-binding portion. For the embodiment, in further more specific embodiments, the multispecific antibody consists of three polypeptide chains, wherein a first polypeptide chain comprises the first antigen-binding portion, the third antigen-binding portion, and one of the Fc polypeptides of the Fc domain described above, a second polypeptide chain comprises the Fab heavy chain of the second antigen-binding portion and the other Fc polypeptide of the Fc domain described above, and a third polypeptide chain is the Fab light chain of the second antigen-binding portion described above.

In some embodiments, the multispecific antibody is trivalent.

In some embodiments, the antibody-drug conjugate is formed by linking a drug-linker having a structure represented by the following formula Ia

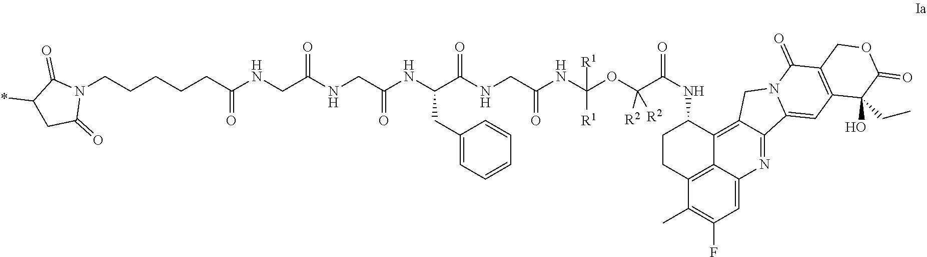

to the multispecific antibody, and the drug-linker is linked to the multispecific antibody at a position indicated by * in the structure represented by formula Ia,
wherein
R¹ and R² are each independently selected from a hydrogen atom and a deuterium atom; preferably, R¹ is a hydrogen atom and R² is a deuterium atom.

The present disclosure provides a method for preparing the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, comprising treating the multispecific antibody targeting EGFR and c-Met under a reducing condition, and then reacting the multispecific antibody targeting EGFR and c-Met with the linker-payload of the present disclosure.

In another aspect, the present disclosure provides a pharmaceutical composition comprising the antibody-drug conjugate, the stereoisomer thereof, the pharmaceutically acceptable salt thereof, or the solvate of the antibody-drug conjugate, the stereoisomer thereof, or the pharmaceutically acceptable salt thereof, and a pharmaceutically acceptable excipient.

In another aspect, the present disclosure provides use of the antibody-drug conjugate, the stereoisomer thereof, the pharmaceutically acceptable salt thereof, or the solvate of the antibody-drug conjugate, the stereoisomer thereof, or the pharmaceutically acceptable salt thereof, or the pharmaceutical composition in preparing a medicament for treating a disease expressing c-Met and/or EGFR.

In another aspect, the present disclosure provides a method for treating a tumor expressing c-Met and/or EGFR, comprising administering to a subject the antibody-drug conjugate, the stereoisomer thereof, the pharmaceutically acceptable salt thereof, or the solvate of the antibody-drug conjugate, the stereoisomer thereof, or the pharmaceutically acceptable salt thereof, or the pharmaceutical composition.

In another aspect, the present disclosure provides the antibody-drug conjugate, the stereoisomer thereof, the pharmaceutically acceptable salt thereof, or the solvate of the antibody-drug conjugate, the stereoisomer thereof, or the pharmaceutically acceptable salt thereof, or the pharmaceutical composition for use as a medicament or for use in treatment.

The antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure targets EGFR and c-Met, and exhibits good anti-tumor effect and/or safety.

### BRIEF DESCRIPTION OF THE DRAWINGS

FIGS. 1A to 1G illustrate binding curves of different anti-human c-Met V_{H}H-Fc chimeric antibodies to target cells with different c-Met expression levels as determined by flow cytometry;

FIG. 2 is a schematic structural view of an exemplary multispecific antibody of the present disclosure;

FIGS. 3A to 3C illustrate binding curves of anti-EGFR/anti-c-Met multispecific antibodies to cells expressing EGFR and c-Met as determined by flow cytometry;

FIG. 4 illustrates the inhibition of c-Met phosphorylation and downstream signaling pathways by anti-EGFR/anti-c-Met multispecific antibodies as determined by Western Blotting, wherein the leftmost lane denotes the marker, the BM denotes amivantamab, the negative denotes hIgG1, the blank denotes no antibody, the HGF “-” or “+” denotes the absence or presence of HGF, respectively, and the p-cMet, p-Akt, p-Erk, and p-EGFR denote phosphorylated c-Met, phosphorylated Akt, phosphorylated Erk, and phosphorylated EGFR, respectively;

FIG. 5 illustrates the inhibition of EGFR phosphorylation and downstream signaling pathways by anti-EGFR/anti-c-Met multispecific antibodies as determined by Western Blotting, wherein the leftmost lane denotes the marker, the BM denotes amivantamab, the negative denotes hIgG1, the blank denotes no antibody, the EGF “-” or “+” denotes the absence or presence of EGF, respectively, and the p-cMet, p-Akt, p-Erk, and p-EGFR denote phosphorylated c-Met, phosphorylated Akt, phosphorylated Erk, and phosphorylated EGFR, respectively;

FIGS. 6A to 6C illustrate curves of anti-EGFR/anti-c-Met multispecific antibodies competing with HGF ligand at different concentrations for binding to c-Met as determined by ELISA;

FIGS. 7A and 7B illustrate the inhibition of proliferation of tumor cells expressing EGFR and c-Met by anti-EGFR/anti-c-Met multispecific antibodies;

FIGS. 8A and 8B illustrate the killing effects of anti-EGFR/anti-c-Met multispecific antibodies on tumor cells expressing EGFR and c-Met;

FIGS. 9A to 9C illustrate the internalization activity of anti-EGFR/anti-c-Met multispecific antibodies in cells expressing EGFR and c-Met as determined by flow cytometry;

FIGS. 10A to 10D illustrate binding curves of V42-DDDXd-6 (1) to cells expressing EGFR and c-Met as determined by flow cytometry, wherein FIG. 10A illustrates the binding curve for HCC827 cells, FIG. 10B illustrates the binding curve for NCI-H292 cells, FIG. 10C illustrates the binding curve for HCA-7 cells, and FIG. 10D illustrates the binding curve for NCI-H441 cells.

FIGS. 11A to 11D illustrate the internalization activity of V42-DDDXd-6 (1) in cells expressing EGFR and c-Met as determined by flow cytometry, wherein FIG. 11A illustrates the internalization activity in HCC827 cells, FIG. 11B illustrates the internalization activity in NCI-H292 cells, FIG. 11C illustrates the internalization activity in HCA-7 cells, and FIG. 11D illustrates the internalization activity in NCI-H441 cells.

FIGS. 12A to 12E illustrate the killing effects of V42-DDDXd-6 (1) on tumor cells expressing EGFR and c-Met, wherein FIG. 12A illustrates the killing effect on HCC827 cells, FIG. 12B illustrates the killing effect on MKN45 cells, FIG. 12C illustrates the killing effect on NCI-H292 cells, FIG. 12D illustrates the killing effect on BxPC3 cells, and FIG. 12E illustrates the killing effect on HCA-7 cells;

FIG. 13 illustrates the bystander effect of V42-DDDXd-6 (1) on EGFR and c-Met-negative cell Jurkat in the presence of HCC827 cells;

FIG. 14 illustrates tumor volume curves of xenograft tumors after administration of V42-DDDXd-6 (1) in NCI-H292 human lung cancer cell nude mouse xenograft tumor model; and

FIG. 15 illustrates tumor volume curves of xenograft tumors after administration of V42-DDDXd-6 (1) in HCA-7 human colon cancer cell nude mouse xenograft tumor model.

### SUMMARY

### Definitions and Description

Unless otherwise stated, the following terms used in the present disclosure shall have the following meanings. A certain term, unless otherwise specifically defined, should not be considered uncertain or unclear, but interpreted according to its common meaning in the art. When referring to a trade name, it is intended to refer to its corresponding commercial product or its active ingredient.

The term “optional” or “optionally” means that the subsequently described event or circumstance may, but does not necessarily, occur. The description includes instances where the event or circumstance occurs and instances where it does not.

The term “substituted” means that any one or more hydrogen atoms on a specific atom are substituted with substituents, as long as the valence of the specific atom is normal and the resulting compound is stable. When the substituent is oxo (i.e., ═O), it means that two hydrogen atoms are substituted; oxo is not possible on an aromatic group. The term “optionally substituted” means that an atom may or may not be substituted with a substituent. Unless otherwise specified, the type and number of the substituent may be arbitrary as long as being chemically achievable.

When any variable (e.g., R) occurs more than once in the constitution or structure of a compound, the definition of the variable in each case is independent. Therefore, for example, if a group is substituted with 2 R, the definition of each R is independent.

The term “sulfydryl” refers to an —SH group.

Unless otherwise stated, the absolute configuration of a stereogenic center is represented by a wedged solid bond ( [symbol] ) and a wedged dashed bond ( [symbol] ).

Unless otherwise specified, when a group has a linkable site, the positions at which the site is linked to other groups may be indicated by an asterisk (*) or a hash symbol (#). For example, the structure of “-(succinimidyl-3-yl-N)-” as used herein is shown in the following formula:

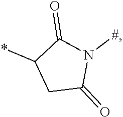

wherein * and #indicate that the carbon atom at position 3 and the nitrogen atom at position 1 of the structure are linked to other groups, respectively.

As used herein, a compound formed by substituting atoms or atom groups in the molecule of the parent compound with other atoms or atom groups is referred to as a “derivative” of the parent compound.

The compounds of the present disclosure may have particular geometric isomeric or stereoisomeric forms. The present disclosure contemplates all such compounds, including cis and trans isomers, levorotatory and dextrorotatory forms, (R)- and(S)-enantiomers, diastereoisomers, (D)-isomers, (L)-isomers, and racemic mixtures and other mixtures thereof, such as enantiomerically or diastereomerically enriched mixtures, all of which fall within the scope of the present disclosure. Additional asymmetric carbon atoms may be present in substituents such as alkyl. All such isomers and mixtures thereof are encompassed within the scope of the present disclosure.

Unless otherwise stated, the term “cis-trans isomer” or “geometric isomer” results from the inability of a double bond or a single bond of a ring carbon atom to rotate freely.

Unless otherwise stated, the term “enantiomer” refers to stereoisomers that are mirror images of each other. Unless otherwise stated, the term “diastereoisomer” refers to stereoisomers whose molecules have two or more chiral centers and are not mirror images of each other.

The compounds and intermediates of the present disclosure may also exist in different tautomeric forms, and all such forms are included within the scope of the present disclosure. The term “tautomer” or “tautomeric form” refers to structural isomers of different energies that can interconvert via a low energy barrier. For example, a proton tautomer (also referred to as prototropic tautomer) includes interconversion via proton transfer, such as keto-enol isomerization and imine-enamine isomerization. A specific example of a proton tautomer is an imidazole moiety where a proton can transfer between two ring nitrogens. A valence tautomer includes the interconversion via recombination of some bonding electrons.

As used herein, the term “bystander effect” refers to an effect in which a cytotoxic drug conjugated to an antibody via a cleavable or non-cleavable linker has the ability to diffuse and penetrate through the cell membrane after release from the antibody and thereby result in killing of adjacent cells. The ability to diffuse and penetrate through the cell membrane is related to the hydrophobicity of the cytotoxic drug or the combination of the cytotoxic drug and the linker. Such cytotoxic drugs may be, for example, an exatecan derivative. The bystander effect may be desirable particularly in tumors with heterogeneous target expression and solid tumors where antibody penetration may be limited.

The term “antibody” is used in its broadest sense and encompasses natural antibodies and artificial antibodies of various structures, including, but not limited to, various antibody structures such as monoclonal antibodies, polyclonal antibodies, multispecific antibodies (e.g., bispecific antibodies or trispecific antibodies), and single-chain antibodies, so long as they exhibit the desired antigen-binding activity.

The term “multispecific” means that the antibody is capable of specifically binding to a plurality of different antigenic determinants, e.g., capable of specifically binding to two or more different antigenic determinants. The antigenic determinant is synonymous with an antigen epitope herein. Generally, a bispecific antibody comprises two antigen-binding sites, each of which is specific for a different antigenic determinant. The different antigenic determinants may be expressed on the same or different cells. The different antigenic determinants may be different due to different antigens (e.g., binding to antigens EGFR and c-Met) or may be present on the same antigen. An antigenic determinant is a special chemical group with a certain composition and structure on the surface or other parts of the antigenic substance molecule, which can specifically bind to its corresponding antibody or sensitized lymphocyte. An example of the antigenic determinant is c-Met, which is an antigenic substance having various structurally defined or structurally undefined antigenic determinants. Antibodies capable of binding to two different antigenic determinants on an antigen are referred to herein as bispecific antibodies. One particular bispecific antibody can, for example, bind to EGFR and c-Met.

The term “N valent antibody” is intended to indicate the presence of N antigen-binding sites in the antibody. For example, “bivalent antibody” or “antibody is bivalent” refers to the presence of two antigen-binding sites in the antibody, and “trivalent antibody” or “antibody is trivalent” refers to the presence of three antigen-binding sites in the antibody. A natural human immunoglobulin molecule typically has two antigen-binding sites, and a Fab typically has a single antigen-binding site. A single variable domain and an scFv typically have a single antigen-binding site.

The term “antigen-binding portion” refers to a polypeptide molecule specifically binding to an antigenic determinant. A specific antigen-binding portion may be a Fab, an scFv, or a single variable domain.

The term “first”, “second”, or “third” used herein with respect to an antigen-binding portion, an antigen, an Fc polypeptide, a peptide linker, a polypeptide chain, and the like is used for ease of distinction when more than one part of each type is present. Unless specifically stated, the use of these terms is not intended to confer a particular order or orientation.

The term “fusion” means that the components (e.g., antigen-binding portions, Fc polypeptides, etc.) are linked either directly or by peptide bonds via one or more peptide linkers. For example, some peptide linkers consist of 1 to 50 amino acids linked by peptide bonds, wherein the amino acids may be selected from 20 naturally occurring amino acids; in a more preferred embodiment, the 1 to 50 amino acids are selected from glycine, alanine, proline, serine, asparagine, glutamine, and lysine.

The term “variable domain” or “variable region” refers to a domain of an antibody that is involved in the binding of the antibody to an antigen. For example, a natural four-chain antibody (e.g., one derived from a human, a murine, etc.) has a heavy chain variable region (also referred to as heavy chain variable domain, VH, or VH domain) and a light chain variable region (also referred to as light chain variable domain, VL, or VL domain), while a heavy-chain antibody derived from an animal such as a camelid or shark has a single variable domain.

In most cases, each variable domain of a natural antibody consists substantially of four “framework regions (FRs)” and three “complementarity determining regions (CDRs)”. The four framework regions are referred to as framework region 1 (or FR1), framework region 2 (or FR2), framework region 3 (or FR3), and framework region 4 (or FR4). The framework regions are separated by three complementarity determining regions referred to in the art and hereinafter as complementarity determining region 1 (or CDR1), complementarity determining region 2 (or CDR2), and complementarity determining region 3 (or CDR3). Thus, the general structure of a variable domain can be represented as follows: FR1-CDR1-FR2-CDR2-FR3-CDR3-FR4. Variable domains impart specificity for an antigen to an antibody by virtue of having an antigen-binding site.

The term “single variable domain” refers to a variable domain capable of specifically binding to an antigen epitope without being paired with other variable domains. The single variable domain typically has three CDRs (CDR1, CDR2, and CDR3) present on a single domain. In some cases, the single variable domain may be a heavy chain variable domain (e.g., VH), so long as it can form a single antigen-binding unit (i.e., a functional antigen-binding unit consisting essentially of a single variable domain; in this way, a single antigen-binding domain can form a functional antigen-binding unit without interacting with another variable domain). Another example of a single variable domain is “V_{H}H domain” of camelids (abbreviated as “V_{H}H” or “V_{H}H”). The term “V_{H}H domain” is used to distinguish these variable domains from the heavy chain variable domains present in conventional four-chain antibodies and the light chain variable domains present in conventional four-chain antibodies. The V_{H}H domain specifically binds to epitopes without additional antigen-binding domains involved (different from the VH or VL domains of conventional four-chain antibodies where VL and VH domains jointly recognize epitopes). The V_{H}H domain is a small, stable, and efficient antigen recognition unit formed from a single domain.

The term “complementarity determining region” (CDR) is also referred to as “hypervariable region” (HVR). A natural four-chain antibody typically comprises six CDRs, among which three are in the heavy chain variable region, i.e., heavy chain CDR1 (HCDR1), heavy chain CDR2 (HCDR2), and heavy chain CDR3 (HCDR3), and three are in the light chain variable region, i.e., light chain CDR1 (LCDR1), light chain CDR2 (LCDR2), and light chain CDR3 (LCDR3). A heavy-chain antibody or a single variable domain typically has three CDRs (CDR1, CDR2, and CDR3).

There are currently many ways to define CDRs. The Kabat scheme defines CDRs based on sequence variability and is the most commonly used scheme. The Chothia scheme defines CDRs based on the positions of structural loops. The AbM scheme, a compromise between the Kabat scheme and the Chothia scheme, is used by the Oxford Molecular's AbM antibody modeling software. The “Contact” defines CDRs based on the analysis of the crystal structure of available complexes. In addition, the CCG definition is also an option. However, it should be noted that boundaries of the CDRs of variable regions of the same antibody defined by different methods may differ, that is, CDR sequences of the variable regions of the same antibody defined by different methods may differ. Thus, when reference is made to an antibody defined by specific CDR sequences, the scope of the antibody also encompasses antibodies defined by CDR sequences under any other definitions (e.g., one of or a combination of several of the definitions such as Kabat, IMGT, Chothia, Contact, AbM, CCG).

The term “Fab” refers to a protein consisting of VH and CH1 domains of the heavy chain and VL and CL domains of the light chain of an immunoglobulin. The Fab herein refers to a Fab molecule in a natural or modified form, i.e., comprising a Fab heavy chain composed of a heavy chain variable region VH and a constant region CH1 (VH-CH1, in a direction from N-terminus to C-terminus), and a Fab light chain composed of a light chain variable region and a constant region CL (VL-CL, in a direction from N-terminus to C-terminus). The modified Fab may be, for example, a Fab in which amino acid substitutions are introduced into the CH1/CL domain or/and the VH/VL domain. In one specific example, the modified Fab may be a Fab in which amino acid substitutions are introduced into the CL domain.

The term “scFv” comprises the VH and VL domains of an immunoglobulin, wherein these domains are present in a single polypeptide chain. In some embodiments, scFv further comprises a peptide linker between the VH and VL domains that enables the scFv to form the required structure for antigen-binding.

The term “Fc domain” or “Fc” is used herein to define the C-terminal region of an immunoglobulin heavy chain, which comprises at least part of the constant region. The term includes natural sequence Fc and variant Fc. The C-terminal lysine (Lys447) of the Fc may or may not be present. Unless otherwise stated, amino acid residues in the Fc or constant region are numbered according to the EU numbering system, also referred to as the EU index, as described in Kabat, E. A. et al., Sequences of Proteins of Immunological Interest, 5th Ed., Public Health Service, National Institutes of Health, Bethesda, MD (1991), NIH Publication 91-3242. As used herein, one of the “Fc polypeptides” of an Fc domain refers to one of the two polypeptides that form a dimeric Fc domain. For example, the Fc polypeptide of an IgG Fc domain comprises IgG CH2 and IgG CH3.

The term “treating” or “treatment” means administering the compound or pharmaceutical composition described herein to prevent, ameliorate, or eliminate a disease or one or more symptoms associated with the disease, including but not limited to: (i) preventing the occurrence of a disease or disease state in a mammal, particularly when such a mammal is predisposed to the disease state but has not yet been diagnosed with it; (ii) inhibiting a disease or disease state, i.e., arresting its progression; (iii) alleviating a disease or disease state, i.e., causing its regression; and (iv) reducing any direct or indirect pathological consequences of a disease or disease state.

The term “therapeutically effective amount” refers to an amount of the compound of the present disclosure for (i) treating or preventing a specific disease, condition, or disorder; (ii) relieving, ameliorating, or eliminating one or more symptoms of a specific disease, condition, or disorder, or (iii) preventing or delaying the onset of one or more symptoms of the specific disease, condition, or disorder described herein. The amount of the antibody-drug conjugate or the pharmaceutical composition of the present disclosure that constitutes a “therapeutically effective amount” may vary depending on factors such as the compound or the pharmaceutical composition and its ability to elicit a desired response in an individual, the disease state and its severity, the mode of administration, and the age, sex, and weight of the mammal to be treated. The therapeutically effective amount may also be determined routinely by those skilled in the art following their knowledge and the present disclosure.

The term “pharmaceutically acceptable” is used herein for those compounds, materials, compositions, and/or dosage forms that are, within the scope of sound medical judgment, suitable for use in contact with the tissues of human beings and animals without excessive toxicity, irritation, allergic response, or other problems or complications, and commensurate with a reasonable benefit/risk ratio.

The term “excipient” refers to any ingredient other than the active ingredient (e.g., the antibody of the present disclosure). The selection of the excipient will largely depend on factors such as the particular mode of administration, the effect of the excipient on solubility and stability, and the nature of the dosage form.

The term “isolated” means that a target compound, such as an antibody or an antigen-binding fragment thereof, V_{H}H, or a nucleic acid, has been isolated from its natural environment.

The terms “Xn” and “Xaa” are equivalent and refer to an unspecified amino acid whose scope is specified by the subsequent definitions in the relevant description.

As used herein, the term “EC₅₀” refers to a concentration of an antibody or ADC that induces 50% of the maximal effective response. The EC₅₀ can be measured by ELISA or FACS analysis or any other method known in the art.

The “K_{D}” refers to the equilibrium dissociation constant, which is derived from the ratio of the dissociation rate constant (k_{d}) to the association rate constant (kₐ) (i.e., k_{d}/kₐ), and is expressed in molar concentration (M). The K_{D} value of an antibody or ADC may be determined using methods well established in the art. A preferred method for determining the K_{D} of an antibody or ADC is surface plasmon resonance (SPR), preferably using a biosensor system such as the Biacore surface plasmon resonance system for analysis.

The term “identity” is also referred to as consistency. The “percent identity (%)” of an amino acid sequence refers to the percentage of amino acid residues in a sequence to be aligned that are identical to those of a specific amino acid sequence as set forth herein when the sequence to be aligned is aligned with the specific amino acid sequence as set forth herein, with gaps introduced, if necessary, to achieve the maximum percent sequence identity and without considering any conservative replacements as part of the sequence identity. The alignment of amino acid sequences for identity can be performed in a variety of ways within the skill in the art. Those skilled in the art can determine suitable parameters for aligning sequences, including any algorithms required to acquire the maximum alignment for the full length of sequences being compared.

The term “subject” includes any human or non-human animal. The term “non-human animal” includes all vertebrates, e.g., mammals and non-mammals, such as non-human primates, sheep, dogs, cats, horses, cows, chickens, amphibians, and reptiles. Preferably, the subject according to the present disclosure is a human. The terms “patient” and “subject” are used interchangeably unless otherwise indicated. The “subjects in need” include those with a disease or condition, those at risk of developing a disease or condition, and those who may have a disease or condition and whose purpose is to prevent, delay, or alleviate the disease or condition.

As used herein, “about” means being within an acceptable error range determined by those of ordinary skill in the art for a specific value, which will depend in part on how the value is measured or determined, i.e., the limitation by the measurement system. For example, “about” may mean being within 1 or more than 1 standard deviation, as practiced in the art. Alternatively, “about” may mean a range of up to ±5%, for example, fluctuating within a specific numerical range given ±2%, ±1%, or ±0.5%. Where a specific value is given in the scope of the present disclosure, unless otherwise stated, “about” should be considered to mean being within an acceptable error range for that specific value. As used herein, unless otherwise stated, the values of the parameters or conditions in a step are all modified by “about” by default.

The terms “comprise”, “comprises”, “comprising”, and equivalents thereof (e.g., contain, contains, containing, include, includes, and including) are to be construed as “including, but not limited to”, meaning that additional unspecified elements, components, and steps may be included in addition to the enumerated elements, components, and steps.

As used herein, unless otherwise specified clearly in the context, singular terms encompass plural referents, and vice versa.

### Antibody-Drug Conjugate (ADC)

The present disclosure provides an antibody-drug conjugate, a stereoisomer thereof, a pharmaceutically acceptable salt thereof, or a solvate of the antibody-drug conjugate, the stereoisomer thereof, or the pharmaceutically acceptable salt thereof, which comprises a multispecific antibody targeting EGFR and c-Met and a cytotoxic drug, wherein the multispecific antibody targeting EGFR and c-Met is conjugated to the cytotoxic drug.

### Multispecific Antibody in ADC

The present disclosure provides an antibody-drug conjugate, a stereoisomer thereof, a pharmaceutically acceptable salt thereof, or a solvate of the antibody-drug conjugate, the stereoisomer thereof, or the pharmaceutically acceptable salt thereof, which comprises a multispecific antibody, wherein the multispecific antibody comprises:
(i) a first antigen-binding portion binding to a first antigen;
(ii) a second antigen-binding portion binding to a second antigen; and
(iii) a third antigen-binding portion binding to the first antigen, wherein the first antigen is c-Met, and the second antigen is EGFR; and the first antigen-binding portion and the third antigen-binding portion are both single variable domains and each independently comprise any one of the following:
(1) a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3:
(2) a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, wherein X₁ is S or T;
(3) a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9;
(4) a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12;
(5) a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15;
(6) a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18;
(7) a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21; or
(8) a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24.

In the present disclosure, the first antigen-binding portion and the third antigen-binding portion are both single variable domains that bind to c-Met. In some embodiments, the first antigen-binding portion comprises the complementarity determining regions described in any one of (1) to (8) above. In some embodiments, the third antigen-binding portion comprises the complementarity determining regions described in any one of (1) to (8) above. In the multispecific antibody, the first antigen-binding portion and the third antigen-binding portion may be identical or different, and any single variable domain described in the present disclosure may be independently selected for combination. In some specific embodiments, the first antigen-binding portion and the third antigen-binding portion are identical. In some other specific embodiments, the first antigen-binding portion and the third antigen-binding portion are different.

Multispecific antibodies constructed with different combinations of the first antigen-binding portion and the third antigen-binding portion with defined CDR features are illustratively listed in Table S1. In some embodiments, the first antigen-binding portion comprises the complementarity determining regions described in (3) above, and the third antigen-binding portion comprises the complementarity determining regions described in (2) above, that is, the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9, and the third antigen-binding portion comprises: a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, wherein X₁ is S or T. In some specific embodiments, the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; and the third antigen-binding portion comprises: a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, wherein X₁ is T. In a more specific embodiment, the first antigen-binding portion comprises the CDR1 set forth in SEQ ID NO: 7, the CDR2 set forth in SEQ ID NO: 8, and the CDR3 set forth in SEQ ID NO: 9; and the third antigen-binding portion comprises the CDR1 set forth in SEQ ID NO: 4, the CDR2 set forth in SEQ ID NO: 5, and the CDR3 set forth in SEQ ID NO: 6, wherein X₁ is T. In some other embodiments, the first antigen-binding portion comprises the complementarity determining regions described in (3) above, and the third antigen-binding portion comprises the complementarity determining regions described in (8) above, that is, the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9, and the third antigen-binding portion comprises: a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24. In a more specific embodiment, the first antigen-binding portion comprises the CDR1 set forth in SEQ ID NO: 7, the CDR2 set forth in SEQ ID NO: 8, and the CDR3 set forth in SEQ ID NO: 9; and the third antigen-binding portion comprises the CDR1 set forth in SEQ ID NO: 22, the CDR2 set forth in SEQ ID NO: 23, and the CDR3 set forth in SEQ ID NO: 24.

TABLE S1
Exemplary multispecific antibodies with defined CDR features in the first
antigen-binding portion and the third antigen-binding portion

| Third antigen- | First antigen-binding portion | | | | | | | |
|---|---|---|---|---|---|---|---|---|
| binding portion | (1) | (2) | (3) | (4) | (5) | (6) | (7) | (8) |
| (1) | (1) + (1) | (1) + (2) | (1) + (3) | (1) + (4) | (1) + (5) | (1) + (6) | (1) + (7) | (1) + (8) |
| (2) | (2) + (1) | (2) + (2) | (2) + (3) | (2) + (4) | (2) + (5) | (2) + (6) | (2) + (7) | (2) + (8) |
| (3) | (3) + (1) | (3) + (2) | (3) + (3) | (3) + (4) | (3) + (5) | (3) + (6) | (3) + (7) | (3) + (8) |
| (4) | (4) + (1) | (4) + (2) | (4) + (3) | (4) + (4) | (4) + (5) | (4) + (6) | (4) + (7) | (4) + (8) |
| (5) | (5) + (1) | (5) + (2) | (5) + (3) | (5) + (4) | (5) + (5) | (5) + (6) | (5) + (7) | (5) + (8) |
| (6) | (6) + (1) | (6) + (2) | (6) + (3) | (6) + (4) | (6) + (5) | (6) + (6) | (6) + (7) | (6) + (8) |
| (7) | (7) + (1) | (7) + (2) | (7) + (3) | (7) + (4) | (7) + (5) | (7) + (6) | (7) + (7) | (7) + (8) |
| (8) | (8) + (1) | (8) + (2) | (8) + (3) | (8) + (4) | (8) + (5) | (8) + (6) | (8) + (7) | (8) + (8) |

Note:
in the table, (X) + (Y) indicates the combination of the third antigen-binding portion and the first antigen-binding portion of the multispecific antibody. For example, (2) + (1) indicates that the third antigen-binding portion of the multispecific antibody comprises the complementarity determining regions described in (2) above, and the first antigen-binding portion comprises the complementarity determining regions described in (1) above.

Further, in some embodiments, the first antigen-binding portion and the third antigen-binding portion each independently comprise an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 25, 26, 27, 28, 29, 30, 31, 32, 33, 34, 35, 36, 37, 38, 39, 40, 41, or 42. In some embodiments, the first antigen-binding portion and the third antigen-binding portion each independently comprise an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 25, 26, 27, 28, 29, 30, 31, 32, 33, 34, 35, 36, 37, 38, or 39.

In some embodiments, the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 25, 26, 27, 28, 29, 30, 31, 32, 33, 34, 35, 36, 37, 38, 39, 40, 41, or 42. In some embodiments, the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 25, 26, 27, 28, 29, 30, 31, 32, 33, 34, 35, 36, 37, 38, or 39. In some specific embodiments, the first antigen-binding portion comprises the amino acid sequence set forth in SEQ ID NO: 25, 26, 27, 28, 29, 30, 31, 32, 33, 34, 35, 36, 37, 38, 39, 40, 41, or 42. In some more specific embodiments, the first antigen-binding portion comprises the amino acid sequence set forth in SEQ ID NO: 25, 26, 27, 28, 29, 30, 31, 32, 33, 34, 35, 36, 37, 38, or 39.

In some embodiments, the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 25, 26, 27, 28, 29, 30, 31, 32, 33, 34, 35, 36, 37, 38, 39, 40, 41, or 42. In some embodiments, the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 25, 26, 27, 28, 29, 30, 31, 32, 33, 34, 35, 36, 37, 38, or 39. In some specific embodiments, the third antigen-binding portion comprises the amino acid sequence set forth in SEQ ID NO: 25, 26, 27, 28, 29, 30, 31, 32, 33, 34, 35, 36, 37, 38, 39, 40, 41, or 42. In some more specific embodiments, the third antigen-binding portion comprises the amino acid sequence set forth in SEQ ID NO: 25, 26, 27, 28, 29, 30, 31, 32, 33, 34, 35, 36, 37, 38, or 39.

In some embodiments, the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 41, and the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 40. In some specific embodiments, the first antigen-binding portion comprises the amino acid sequence set forth in SEQ ID NO: 41, and the third antigen-binding portion comprises the amino acid sequence set forth in SEQ ID NO: 40. In some more specific embodiments, the first antigen-binding portion comprises the amino acid sequence set forth in SEQ ID NO: 27, 36, or 37, and the third antigen-binding portion comprises the amino acid sequence set forth in SEQ ID NO: 26, 33, 34, or 35.

In some embodiments, the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 41, and the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 42. In some specific embodiments, the first antigen-binding portion comprises the amino acid sequence set forth in SEQ ID NO: 41, and the third antigen-binding portion comprises the amino acid sequence set forth in SEQ ID NO: 42. In some more specific embodiments, the first antigen-binding portion comprises the amino acid sequence set forth in SEQ ID NO: 27, 36, or 37, and the third antigen-binding portion comprises the amino acid sequence set forth in SEQ ID NO: 32, 38, or 39.

In one example, the first antigen-binding portion comprises the amino acid sequence set forth in SEQ ID NO: 27, and the third antigen-binding portion comprises the amino acid sequence set forth in SEQ ID NO: 26, 32, 33, 34, 35, 38, or 39.

In one example, the third antigen-binding portion comprises the amino acid sequence set forth in SEQ ID NO: 27, and the first antigen-binding portion comprises the amino acid sequence set forth in SEQ ID NO: 26, 32, 33, 34, 35, 38, or 39.

In one example, the first antigen-binding portion comprises the amino acid sequence set forth in SEQ ID NO: 36, and the third antigen-binding portion comprises the amino acid sequence set forth in SEQ ID NO: 26, 32, 33, 34, 35, 38, or 39.

In one example, the third antigen-binding portion comprises the amino acid sequence set forth in SEQ ID NO: 36, and the first antigen-binding portion comprises the amino acid sequence set forth in SEQ ID NO: 26, 32, 33, 34, 35, 38, or 39.

In one example, the first antigen-binding portion comprises the amino acid sequence set forth in SEQ ID NO: 37, and the third antigen-binding portion comprises the amino acid sequence set forth in SEQ ID NO: 26, 32, 33, 34, 35, 38, or 39.

In one example, the third antigen-binding portion comprises the amino acid sequence set forth in SEQ ID NO: 37, and the first antigen-binding portion comprises the amino acid sequence set forth in SEQ ID NO: 26, 32, 33, 34, 35, 38, or 39.

In one example, the first antigen-binding portion comprises the amino acid sequence set forth in SEQ ID NO: 27, and the third antigen-binding portion comprises the amino acid sequence set forth in SEQ ID NO: 26.

In one example, the third antigen-binding portion comprises the amino acid sequence set forth in SEQ ID NO: 27, and the first antigen-binding portion comprises the amino acid sequence set forth in SEQ ID NO: 26.

In one example, the first antigen-binding portion comprises the amino acid sequence set forth in SEQ ID NO: 36, and the third antigen-binding portion comprises the amino acid sequence set forth in SEQ ID NO: 33.

In one example, the third antigen-binding portion comprises the amino acid sequence set forth in SEQ ID NO: 36, and the first antigen-binding portion comprises the amino acid sequence set forth in SEQ ID NO: 33.

In one example, the first antigen-binding portion comprises the amino acid sequence set forth in SEQ ID NO: 36, and the third antigen-binding portion comprises the amino acid sequence set forth in SEQ ID NO: 34.

In one example, the third antigen-binding portion comprises the amino acid sequence set forth in SEQ ID NO: 36, and the first antigen-binding portion comprises the amino acid sequence set forth in SEQ ID NO: 34.

In one example, the first antigen-binding portion comprises the amino acid sequence set forth in SEQ ID NO: 36, and the third antigen-binding portion comprises the amino acid sequence set forth in SEQ ID NO: 35.

In one example, the third antigen-binding portion comprises the amino acid sequence set forth in SEQ ID NO: 36, and the first antigen-binding portion comprises the amino acid sequence set forth in SEQ ID NO: 35.

In one example, the first antigen-binding portion comprises the amino acid sequence set forth in SEQ ID NO: 36, and the third antigen-binding portion comprises the amino acid sequence set forth in SEQ ID NO: 32.

In one example, the third antigen-binding portion comprises the amino acid sequence set forth in SEQ ID NO: 36, and the first antigen-binding portion comprises the amino acid sequence set forth in SEQ ID NO: 32.

In one example, the first antigen-binding portion comprises the amino acid sequence set forth in SEQ ID NO: 36, and the third antigen-binding portion comprises the amino acid sequence set forth in SEQ ID NO: 38.

In one example, the third antigen-binding portion comprises the amino acid sequence set forth in SEQ ID NO: 36, and the first antigen-binding portion comprises the amino acid sequence set forth in SEQ ID NO: 38.

In one example, the first antigen-binding portion comprises the amino acid sequence set forth in SEQ ID NO: 36, and the third antigen-binding portion comprises the amino acid sequence set forth in SEQ ID NO: 39.

In one example, the third antigen-binding portion comprises the amino acid sequence set forth in SEQ ID NO: 36, and the first antigen-binding portion comprises the amino acid sequence set forth in SEQ ID NO: 39.

In one example, the first antigen-binding portion and the third antigen-binding portion both comprise the amino acid sequence set forth in SEQ ID NO: 25, 26, 27, 28, 29, 30, 31, 32, 33, 34, 35, 36, 37, 38, or 39.

Multispecific antibodies constructed with different combinations of the first antigen-binding portion and the third antigen-binding portion with full-length amino acid sequence features are illustratively listed in Table S2.

TABLE S2
Exemplary multispecific antibodies with defined amino acid sequences in the first antigen-binding
portion and the third antigen-binding portion

| | First antigen-binding portion | | | | | | |
|---|---|---|---|---|---|---|---|
| | 1B-1B6-V1 | 1B-1B6-V2 | 1B-1B6-V3 | 1B-1C7-V1 | 1B-1C7-V2 | 12B04-V1 | 12B04-V2 |
| Third | single | single | single | single | single | single | single |
| antigen-binding | variable | variable | variable | variable | variable | variable | variable |
| portion | domain | domain | domain | domain | domain | domain | domain |
| 1B-1B6-V1 single | 1B-1B6-V1 + | 1B-1B6-V1 + | 1B-1B6-V1 + | 1B-1B6-V1 + | 1B-1B6-V1 + | 1B-1B6-V1 + | 1B-1B6-V1 + |
| variable domain | 1B-1B6- | 1B-1B6- | 1B-1B6- | 1B-1C7- | 1B-1C7- | 12B04- | 12B04-V2 |
| | V1 single | V2 single | V3 single | V1 single | V2 single | V1 single | single |
| | variable | variable | variable | variable | variable | variable | variable |
| | domains | domains | domains | domains | domains | domains | domains |
| 1B-1B6-V2 single | 1B-1B6-V2 + | 1B-1B6-V2 + | 1B-1B6-V2 + | 1B-1B6-V2 + | 1B-1B6-V2 + | 1B-1B6-V2 + | 1B-1B6-V2 + |
| variable domain | 1B-1B6- | 1B-1B6- | 1B-1B6- | 1B-1C7- | 1B-1C7- | 12B04- | 12B04-V2 |
| | V1 single | V2 single | V3 single | V1 single | V2 single | V1 single | single |
| | variable | variable | variable | variable | variable | variable | variable |
| | domains | domains | domains | domains | domains | domains | domains |
| 1B-1B6-V3 single | 1B-1B6-V3 + | 1B-1B6-V3 + | 1B-1B6-V3 + | 1B-1B6-V3 + | 1B-1B6-V3 + | 1B-1B6-V3 + | 1B-1B6-V3 + |
| variable domain | 1B-1B6- | 1B-1B6- | 1B-1B6- | 1B-1C7- | 1B-1C7- | 12B04- | 12B04-V2 |
| | V1 single | V2 single | V3 single | V1 single | V2 single | V1 single | single |
| | variable | variable | variable | variable | variable | variable | variable |
| | domains | domains | domains | domains | domains | domains | domains |
| 1B-1C7-V1 single | 1B-1C7-V1 + | 1B-1C7-V1 + | 1B-1C7-V1 + | 1B-1C7-V1 + | 1B-1C7-V1 + | 1B-1C7-V1 + | 1B-1C7-V1 + |
| variable domain | 1B-1B6- | 1B-1B6- | 1B-1B6- | 1B-1C7- | 1B-1C7- | 12B04- | 12B04-V2 |
| | V1 single | V2 single | V3 single | V1 single | V2 single | V1 single | single |
| | variable | variable | variable | variable | variable | variable | variable |
| | domains | domains | domains | domains | domains | domains | domains |
| 1B-1C7-V2 single | 1B-1C7-V2 + | 1B-1C7-V2 + | 1B-1C7-V2 + | 1B-1C7-V2 + | 1B-1C7-V2 + | 1B-1C7-V2 + | 1B-1C7-V2 + |
| variable domain | 1B-1B6- | 1B-1B6- | 1B-1B6- | 1B-1C7- | 1B-1C7- | 12B04- | 12B04-V2 |
| | V1 single | V2 single | V3 single | V1 single | V2 single | V1 single | single |
| | variable | variable | variable | variable | variable | variable | variable |
| | domains | domains | domains | domains | domains | domains | domains |
| 12B04-V1 single | 12B04-V1 + | 12B04-V1 + | 12B04-V1 + | 12B04-V1 + | 12B04-V1 + | 12B04-V1 + | 12B04-V1 + |
| variable domain | 1B-1B6- | 1B-1B6- | 1B-1B6- | 1B-1C7- | 1B-1C7- | 12B04- | 12B04-V2 |
| | V1 single | V2 single | V3 single | V1 single | V2 single | V1 single | single |
| | variable | variable | variable | variable | variable | variable | variable |
| | domains | domains | domains | domains | domains | domains | domains |
| 12B04-V2 single | 12B04-V2 + | 12B04-V2 + | 12B04-V2 + | 12B04-V2 + | 12B04-V2 + | 12B04-V2 + | 12B04-V2 + |
| variable domain | 1B-1B6- | 1B-1B6- | 1B-1B6- | 1B-1C7- | 1B-1C7- | 12B04- | 12B04-V2 |
| | V1 single | V2 single | V3 single | V1 single | V2 single | V1 single | single |
| | variable | variable | variable | variable | variable | variable | variable |
| | domains | domains | domains | domains | domains | domains | domains |

Note:
in the table, (X) + (Y) indicates the combination of the third antigen-binding portion and the first antigen-binding portion of the multispecific antibody. For example, “1B-1B6-V1 + 1B-1C7-V1 single variable domains” indicates that the third antigen-binding portion of the multispecific antibody comprises the amino acid sequence of the 1B-1B6-V1 single variable domain, and the first antigen-binding portion comprises the amino acid sequence of the 1B-1C7-V1 single variable domain; the amino acid sequences of the single variable domains referred to in the table are shown in Table S3.

TABLE S3
CDRs and full-length amino acid sequences of
single variable domains (SEQ ID NO.)

| | | | | Full |
|---|---|---|---|---|
| Name | CDR1 | CDR2 | CDR3 | length |
| 1B-3B11 single | 1 | 2 | 3 | 25 |
| variable domain | | | | |
| 1B-1B6 single | 4 | 5, X₁ is S | 6 | 26 |
| variable domain | | | | |
| 1B-1B6-V1 single | | | | 33 |
| variable domain | | | | |
| 1B-1B6-V2 single | | | | 34 |
| variable domain | | | | |
| 1B-1B6-V3 single | 4 | 5, X₁ is T | 6 | 35 |
| variable domain | | | | |
| 1B-1C7 single | 7 | | | 27 |
| variable domain | | | | |
| 1B-1C7-V1 single | | 8 | 9 | 36 |
| variable domain | | | | |
| 1B-1C7-V2 single | | | | 37 |
| variable domain | | | | |
| 1B-1A8 single | 10 | 11 | 12 | 28 |
| variable domain | | | | |
| 1B-1B2 single | 13 | 14 | 15 | 29 |
| variable domain | | | | |
| 3B-1C7 single | 16 | 17 | 18 | 30 |
| variable domain | | | | |
| 4&5B-2F01 single | 19 | 20 | 21 | 31 |
| variable domain | | | | |
| 4&5C-12B04 single | 22 | 23 | | 32 |
| variable domain | | | | |
| 12B04-V1 single | | | 24 | 38 |
| variable domain | | | | |
| 12B04-V2 single | | | | 39 |
| variable domain | | | | |

In the present disclosure, the first antigen-binding portion may be derived from a camelid or be humanized. The third antigen-binding portion may be derived from a camelid or be humanized. Humanization may reduce the immunogenicity, and in some embodiments, both the first antigen-binding portion and the third antigen-binding portion are humanized. In some embodiments, both the first antigen-binding portion and the third antigen-binding portion are derived from a camelid.

In the multispecific antibody of the present disclosure, the second antigen-binding portion provides the ability to target EGFR. The second antigen-binding portion may be a Fab, an scFv, or an ScFab (single-chain Fab). In some embodiments, the second antigen-binding portion is a Fab binding to EGFR.

In some embodiments, the second antigen-binding portion is murine, chimeric, or humanized.

In some embodiments, the second antigen-binding portion comprises: an HCDR1 comprising the amino acid sequence set forth in SEQ ID NO: 63, an HCDR2 comprising the amino acid sequence set forth in SEQ ID NO: 64, an HCDR3 comprising the amino acid sequence set forth in SEQ ID NO: 65, an LCDR1 comprising the amino acid sequence set forth in SEQ ID NO: 66, an LCDR2 comprising the amino acid sequence set forth in SEQ ID NO: 67, and an LCDR3 comprising the amino acid sequence set forth in SEQ ID NO: 68. In some embodiments, the second antigen-binding portion comprises: an HCDR1 having the amino acid sequence set forth in SEQ ID NO: 63, an HCDR2 having the amino acid sequence set forth in SEQ ID NO: 64, an HCDR3 having the amino acid sequence set forth in SEQ ID NO: 65, an LCDR1 having the amino acid sequence set forth in SEQ ID NO: 66, an LCDR2 having the amino acid sequence set forth in SEQ ID NO: 67, and an LCDR3 having the amino acid sequence set forth in SEQ ID NO: 68. In some embodiments, the second antigen-binding portion comprises a heavy chain variable region and a light chain variable region, wherein the heavy chain variable region comprises: an HCDR1 comprising the amino acid sequence set forth in SEQ ID NO: 63, an HCDR2 comprising the amino acid sequence set forth in SEQ ID NO: 64, and an HCDR3 comprising the amino acid sequence set forth in SEQ ID NO: 65, and the light chain variable region comprises: an LCDR1 comprising the amino acid sequence set forth in SEQ ID NO: 66, an LCDR2 comprising the amino acid sequence set forth in SEQ ID NO: 67, and an LCDR3 comprising the amino acid sequence set forth in SEQ ID NO: 68. In some embodiments, the second antigen-binding portion comprises a heavy chain variable region and a light chain variable region, wherein the heavy chain variable region comprises: an HCDR1 having the amino acid sequence set forth in SEQ ID NO: 63, an HCDR2 having the amino acid sequence set forth in SEQ ID NO: 64, and an HCDR3 having the amino acid sequence set forth in SEQ ID NO: 65, and the light chain variable region comprises: an LCDR1 having the amino acid sequence set forth in SEQ ID NO: 66, an LCDR2 having the amino acid sequence set forth in SEQ ID NO: 67, and an LCDR3 having the amino acid sequence set forth in SEQ ID NO: 68.

In one specific embodiment, the second antigen-binding portion comprises: an HCDR1, an HCDR2, and an HCDR3 in a heavy chain variable region having the amino acid sequence set forth in SEQ ID NO: 69, and an LCDR1, an LCDR2, and an LCDR3 in a light chain variable region having the amino acid sequence set forth in SEQ ID NO: 70. In one specific embodiment, the heavy chain variable region comprises the HCDR1, the HCDR2, and the HCDR3 of the variable region set forth in SEQ ID NO: 69, and the light chain variable region comprises the LCDR1, the LCDR2, and the LCDR3 of the variable region set forth in SEQ ID NO: 70. The CDRs are defined, for example, according to Kabat, IMGT, Chothia, Contact, AbM, and/or CCG.

In some embodiments, the second antigen-binding portion comprises a heavy chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 69, and a light chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 70. In some embodiments, the heavy chain variable region of the second antigen-binding portion comprises the amino acid sequence set forth in SEQ ID NO: 69, and the light chain variable region of the second antigen-binding portion comprises the amino acid sequence set forth in SEQ ID NO: 70. In some embodiments, for the second antigen-binding portion, the amino acid sequence of the heavy chain variable region is set forth in SEQ ID NO: 69, and the amino acid sequence of the light chain variable region is set forth in SEQ ID NO: 70.

The multispecific antibody of the present disclosure may have no Fc domain, and the first antigen-binding portion, the second antigen-binding portion, and the third antigen-binding portion are fused via suitable linkers. The multispecific antibody of the present disclosure may have an Fc domain, and the Fc domain may prolong the half-life, provide the Fc domain-related effector, and the like.

### Multispecific Antibody Configurations

The antigen-binding portions in the multispecific antibody of the present disclosure may be fused to one another in a variety of configurations. In some embodiments, the multispecific antibody further comprises (iv) an Fc domain composed of two Fc polypeptides.

In some embodiments, the third antigen-binding portion and the first antigen-binding portion are fused to each other, optionally via a peptide linker. In some more specific embodiments, the third antigen-binding portion, at the C-terminus, is fused to the N-terminus of the first antigen-binding portion. Further, in some embodiments, the first antigen-binding portion, at the C-terminus, is fused to the N-terminus of one of the Fc polypeptides of the Fc domain, the second antigen-binding portion is a Fab, and the second antigen-binding portion, at the C-terminus of the Fab heavy chain or the Fab light chain, is fused to the N-terminus of the other Fc polypeptide of the Fc domain.

In one specific embodiment, the first antigen-binding portion and the third antigen-binding portion are both single variable domains, and the second antigen-binding portion is a Fab; the first antigen-binding portion, at the C-terminus, is fused to the N-terminus of one of the Fc polypeptides of the Fc domain, the second antigen-binding portion, at the C-terminus of the Fab heavy chain, is fused to the N-terminus of the other Fc polypeptide of the Fc domain, and the third antigen-binding portion, at the C-terminus, is fused to the N-terminus of the first antigen-binding portion. Such configurations are schematically depicted in FIG. 2. For the embodiment, in further more specific embodiments, the multispecific antibody has three polypeptide chains, wherein a first polypeptide chain comprises the first antigen-binding portion, the third antigen-binding portion, and one of the Fc polypeptides of the Fc domain described above, a second polypeptide chain comprises the Fab heavy chain of the second antigen-binding portion and the other Fc polypeptide of the Fc domain described above, and a third polypeptide chain is the Fab light chain of the second antigen-binding portion described above.

In the embodiments described above, the antigen-binding portions of the multispecific antibody are operably linked, e.g., fused directly or via various peptide linkers (e.g., peptide linkers comprising one or more amino acids, typically about 1 to 50 amino acids) or hinges as described herein or known in the art, which can be properly selected by those skilled in the art.

The third antigen-binding portion may be fused to the first antigen-binding portion either directly or via a peptide linker. In one embodiment, the third antigen-binding portion is fused to the first antigen-binding portion via a peptide linker.

The peptide linkers may be each independently any suitable, e.g., electrically charged and/or flexible, linker polypeptide. In specific embodiments, the peptide linker consists of 1 to 50 amino acids linked by peptide bonds, wherein the amino acids may be selected from 20 naturally occurring amino acids; in a more preferred embodiment, the 1 to 50 amino acids are selected from glycine, alanine, proline, serine, asparagine, glutamine, and lysine. Thus, exemplary peptide linkers may be polyglycines (particularly (Gly) 4 and (Gly) 5), poly(Gly-Ser), (Gly)₃AsnGlySer (Gly)₂, (Gly)₃Cys (Gly)₄, GlyProAsnGlyGly, or those disclosed in Table 4 of Patent No. WO2019195535, etc.

In some embodiments, the peptide linker may be a peptide linker consisting of glycine and serine. In some embodiments, the peptide linker may comprise 0, 1, 2, 3, 4, 5, 6, 7, 8, 9, 10, 11, 12, 13, 14, 15, 16, 17, 18, 19, 20, or more amino acids. In some embodiments, the peptide linker is a peptide linker comprising GGGGS as a unit. In some embodiments, the peptide linker comprising GGGGS as a unit is (GGGGS) n, wherein n is any number between 1 and 10, i.e., 1, 2, 3, 4, 5, 6, 7, 8, 9, or 10, or any range defined by any two of the aforementioned numbers, e.g., 1 to 5, 2 to 5, 3 to 6, 2 to 4, or 1 to 4. In some specific embodiments, the peptide linker is a linker polypeptide comprising GGGGS (SEQ ID NO: 91), (GGGGS) 2, (GGGGS)₃, or (GGGGS) 4. In some specific embodiments, the third antigen-binding portion is fused to the first antigen-binding portion via a peptide linker GGGGS (SEQ ID NO: 91) or (GGGGS) 2.

When fused to an Fc, it is typically fused via a hinge region. In one embodiment, the first antigen-binding portion is fused to one of the Fc polypeptides of the Fc domain via a first hinge, and the second antigen-binding portion is fused to the other Fc polypeptide of the Fc domain via a second hinge. In some embodiments, the first hinge and the second hinge can form a covalent bond, such as a disulfide bond. The first hinge or/and the second hinge may comprise amino acids of the hinge region of a human IgG, and the hinge region of the human IgG comprises a natural hinge region or a variant thereof. In some embodiments, the first hinge or/and the second hinge comprises amino acids of the hinge region of a human IgG1. In some embodiments, the first hinge or/and the second hinge comprises amino acids of the hinge region of a human IgG4. In some specific embodiments, the first hinge comprises GEPKSSDKTHTCPPCP (SEQ ID NO: 89), and the second hinge comprises EPKSCDKTHTCPPCP (SEQ ID NO: 90).

### Fc Domain of Multispecific Antibody

The Fc domain of the multispecific antibody consists of a pair of polypeptide chains comprising a heavy chain domain of an immunoglobulin molecule. For example, the Fc domain of immunoglobulin G (IgG) molecule is a dimer, and each of the Fc polypeptides comprises CH2 and CH3 of the IgG heavy chain constant regions. The two Fc polypeptides of the Fc domain are capable of stable association with each other. The C-terminal lysine of the Fc polypeptide may be present or deleted. In one embodiment, the multispecific antibody of the present disclosure comprises one Fc domain.

In some embodiments, the Fc domain of the multispecific antibody is an IgG Fc domain. In some embodiments, the Fc domain is an IgG1 Fc domain. In some embodiments, the Fc domain is a human Fc domain. In some specific embodiments, the Fc domain is a human IgG1 Fc domain.

In some embodiments, the Fc domain comprises a modification, such as an amino acid substitution. The modification may be, for example, a modification that promotes heterodimerization, a modification that alters the binding ability to Protein A, etc.

In some embodiments, the Fc comprises a modification that promotes heterodimerization.

The multispecific antibody of the present disclosure comprises different antigen-binding portions fused to one or the other of the two Fc polypeptides in the Fc domain. Therefore, the two Fc polypeptides are typically comprised in two different polypeptide chains. Several possible combinations of the two polypeptides are generated by recombinant co-expression and subsequent dimerization of these polypeptides. In order to increase the yield and purity of the multispecific antibody in recombinant production, it would be advantageous to introduce a modification that promotes the binding of the desired polypeptides into the Fc domain of the multispecific antibody. Thus, in specific embodiments, the Fc domain comprises an amino acid substitution that promotes the association of the two Fc polypeptides of the Fc domain.

The site for the most extensive protein-protein interaction between the two Fc polypeptides of the human IgG Fc domain is in the CH3 domain of the Fc domain. Thus, in one embodiment, the modification is in the CH3 domain of the Fc domain.

In specific embodiments, the modification is a so-called “knob-into-hole” modification, which comprises a “knob” modification in one of the two Fc polypeptides of the Fc domain and a “hole” modification in the other one of the two Fc polypeptides of the Fc domain. Generally, the method involves introducing a protuberance (“knob”) at the interface of one Fc polypeptide and a corresponding cavity (“hole”) at the interface of the other Fc polypeptide, such that the protuberance can be positioned in the cavity to promote heterodimer formation and to interfere with homodimer formation. The protuberance is constructed by substituting a small amino acid side chain from the interface of one Fc polypeptide with a larger side chain (e.g., tyrosine, tryptophan, etc.). The complementary cavity having the same or similar size as the protuberance is produced in the interface of the other Fc polypeptide by substituting a large amino acid side chain with a smaller amino acid side chain (e.g., alanine or threonine, etc.).

Thus, in specific embodiments, an amino acid residue is substituted with an amino acid residue having a larger side chain volume in the CH3 domain of one Fc polypeptide of the multispecific antibody, thereby producing a protuberance within the CH3 domain of the Fc polypeptide that can be positioned in a cavity within the CH3 domain of the other Fc polypeptide, and an amino acid residue is substituted with an amino acid residue having a smaller side chain volume in the CH3 domain of the other Fc polypeptide, thereby producing a cavity within the CH3 domain of the Fc polypeptide.

In some embodiments, according to the EU numbering, one Fc polypeptide of the Fc domain comprises amino acid substitutions 354C or/and 366Y/W, and the other Fc polypeptide comprises amino acid substitutions 349C, 366S, 368A, or/and 407T/V. In some specific embodiments, according to the EU numbering, one of the Fc polypeptides of the Fc domain comprises amino acid substitutions 354C and 366Y/W, and the other Fc polypeptide comprises amino acid substitutions 349C, 366S, 368A, and 407T/V. In some more specific embodiments, according to the EU numbering, one of the Fc polypeptides of the Fc domain comprises amino acid substitutions 354C and 366W, and the other Fc polypeptide comprises amino acid substitutions 349C, 366S, 368A, and 407V. In the above embodiments, the Fc may be an Fc of a human IgG1. In one specific embodiment, according to the EU numbering, one of the Fc polypeptides of the Fc domain comprises amino acid substitutions S354C and T366W, and the other Fc polypeptide comprises amino acid substitutions Y349C, T366S, L368A, and Y407V.

In some embodiments, the Fc domain comprises a modification that reduces or eliminates the binding of the CH3 region of one Fc polypeptide in the Fc domain to Protein A (from Staphylococcus aureus). In some embodiments, the Fc domain comprises an amino acid substitution that reduces or eliminates the binding of the CH3 region of one Fc polypeptide in the Fc domain to Protein A. In some embodiments, according to the EU numbering, the Fc domain comprises amino acid substitution(s): (a) 435R or (b) 435R and 436F that occur only in one Fc polypeptide but not in the other Fc polypeptide. In some embodiments, according to the EU numbering, the Fc domain comprises amino acid substitution(s): (a) 435R or (b) 435R and 436F that occur only in one of the Fc polypeptides. In some specific embodiments, according to the EU numbering, the Fc domain comprises amino acid substitutions H435R and Y436F that occur only in one of the Fc polypeptides. In some specific embodiments, according to the EU numbering, the Fc domain comprises an amino acid substitution H435R that occurs only in one of the Fc polypeptides. In the above embodiment, the Fc is an IgG1 Fc, particularly a human IgG1 Fc.

In the multispecific antibody of the present disclosure, the Fc domain may comprise (i) a modification that promotes heterodimerization and/or (ii) a modification that reduces or eliminates the binding of the CH3 region of one Fc polypeptide in the Fc domain to Protein A. In some embodiments, the Fc comprises (i) a modification that promotes heterodimerization and (ii) a modification that reduces or eliminates the binding of the CH3 region of one Fc polypeptide in the Fc domain to Protein A. For example, in one specific embodiment, according to the EU numbering, the Fc domain comprises the following groups of amino acid substitutions:
i. 349C, 366S, 368A, 407T/V, 354C, and 366Y/W, wherein amino acid substitutions 354C and 366Y/W are on the same Fc polypeptide, and are not on the same Fc polypeptide as the other amino acid substitutions in (i); and
ii. (a) 435R or (b) 435R and 436F that occur only in one of the Fc polypeptides.

In one more specific embodiment, according to the EU numbering, one of the Fc polypeptides of the Fc domain comprises amino acid substitutions Y349C, T366S, L368A, Y407V, H435R, and Y436F, and the other Fc polypeptide comprises amino acid substitutions S354C and T366W. In such a specific embodiment, the Fc is an IgG1 Fc, particularly a human IgG1 Fc.

In another more specific embodiment, according to the EU numbering, one of the Fc polypeptides of the Fc domain comprises amino acid substitutions Y349C, T366S, L368A, Y407V, and H435R, and the other Fc polypeptide comprises amino acid substitutions S354C and T366W. In such a specific embodiment, the Fc is an IgG1 Fc, particularly a human IgG1 Fc.

In the context of the present disclosure, amino acid substitutions are represented as: original amino acid-position-substituted amino acid, where the amino acid residues are represented by three-letter (Xaa) or single-letter (X) codes, and the original amino acid can be omitted. Thus, for example, the “H435R” or “435R” means that amino acid H or the original amino acid at position 435 is substituted with amino acid R. More than 1 substituted amino acid may be contained. For example, T366Y/W means that amino acid T at position 366 is substituted with amino acid Y or W.

In some embodiments, the Fc domain does not comprise fucose.

In some embodiments, the multispecific antibody of the present disclosure is afucosylated. The interaction of the multispecific antibody with FcγRIIIa can be improved by defucosylation, thereby enhancing the ADCC effect of the antibody. Methods of producing multispecific antibodies with little or no fucose at the glycosylation site in the Fc domain without altering the amino acid sequence are known in the art, for example, adjusting the composition of the medium containing the expressing cells, or knocking out fucose expression-related genes such as FUT8 in the expressing cells.

In some embodiments, the multispecific antibody described in the present disclosure is trivalent, that is, the first antigen-binding portion, the second antigen-binding portion, and the third antigen-binding portion each provide monovalent binding to the corresponding antigen.

In some embodiments, the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 71, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

In some embodiments, the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 73, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

In some embodiments, the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 75, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

In some embodiments, the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 77, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

In some embodiments, the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 79, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

In some embodiments, the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 81, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

In some embodiments, the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 83, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

In some embodiments, the multispecific antibody consists of three polypeptide chains, wherein one polypeptide chain comprises the amino acid sequence set forth in SEQ ID NO: 71, another polypeptide chain comprises the amino acid sequence set forth in SEQ ID NO: 85, and the other polypeptide chain comprises the amino acid sequence set forth in SEQ ID NO: 87.

In some embodiments, the multispecific antibody consists of three polypeptide chains, wherein one polypeptide chain comprises the amino acid sequence set forth in SEQ ID NO: 73, another polypeptide chain comprises the amino acid sequence set forth in SEQ ID NO: 85, and the other polypeptide chain comprises the amino acid sequence set forth in SEQ ID NO: 87.

In some embodiments, the multispecific antibody consists of three polypeptide chains, wherein one polypeptide chain comprises the amino acid sequence set forth in SEQ ID NO: 75, another polypeptide chain comprises the amino acid sequence set forth in SEQ ID NO: 85, and the other polypeptide chain comprises the amino acid sequence set forth in SEQ ID NO: 87.

In some embodiments, the multispecific antibody consists of three polypeptide chains, wherein one polypeptide chain comprises the amino acid sequence set forth in SEQ ID NO: 77, another polypeptide chain comprises the amino acid sequence set forth in SEQ ID NO: 85, and the other polypeptide chain comprises the amino acid sequence set forth in SEQ ID NO: 87.

In some embodiments, the multispecific antibody consists of three polypeptide chains, wherein one polypeptide chain comprises the amino acid sequence set forth in SEQ ID NO: 79, another polypeptide chain comprises the amino acid sequence set forth in SEQ ID NO: 85, and the other polypeptide chain comprises the amino acid sequence set forth in SEQ ID NO: 87.

In some embodiments, the multispecific antibody consists of three polypeptide chains, wherein one polypeptide chain comprises the amino acid sequence set forth in SEQ ID NO: 81, another polypeptide chain comprises the amino acid sequence set forth in SEQ ID NO: 85, and the other polypeptide chain comprises the amino acid sequence set forth in SEQ ID NO: 87.

In some embodiments, the multispecific antibody consists of three polypeptide chains, wherein one polypeptide chain comprises the amino acid sequence set forth in SEQ ID NO: 83, another polypeptide chain comprises the amino acid sequence set forth in SEQ ID NO: 85, and the other polypeptide chain comprises the amino acid sequence set forth in SEQ ID NO: 87.

The present disclosure provides an exemplary trivalent multispecific antibody.

In one example, the multispecific antibody consists of three polypeptide chains, and the amino acid sequences of the three polypeptide chains are set forth in SEQ ID NO: 71, SEQ ID NO: 85, and SEQ ID NO: 87, respectively. In some embodiments, the nucleotide sequences encoding the three polypeptide chains are set forth in SEQ ID NO: 72, SEQ ID NO: 86, and SEQ ID NO: 88, respectively.

In one example, the multispecific antibody consists of three polypeptide chains, and the amino acid sequences of the three polypeptide chains are set forth in SEQ ID NO: 73, SEQ ID NO: 85, and SEQ ID NO: 87, respectively. In some embodiments, the nucleotide sequences encoding the three polypeptide chains are set forth in SEQ ID NO: 74, SEQ ID NO: 86, and SEQ ID NO: 88, respectively.

In one example, the multispecific antibody consists of three polypeptide chains, and the amino acid sequences of the three polypeptide chains are set forth in SEQ ID NO: 75, SEQ ID NO: 85, and SEQ ID NO: 87, respectively. In some embodiments, the nucleotide sequences encoding the three polypeptide chains are set forth in SEQ ID NO: 76, SEQ ID NO: 86, and SEQ ID NO: 88, respectively.

In one example, the multispecific antibody consists of three polypeptide chains, and the amino acid sequences of the three polypeptide chains are set forth in SEQ ID NO: 77, SEQ ID NO: 85, and SEQ ID NO: 87, respectively.

In some embodiments, the nucleotide sequences encoding the three polypeptide chains are set forth in SEQ ID NO: 78, SEQ ID NO: 86, and SEQ ID NO: 88, respectively.

In one example, the multispecific antibody consists of three polypeptide chains, and the amino acid sequences of the three polypeptide chains are set forth in SEQ ID NO: 79, SEQ ID NO: 85, and SEQ ID NO: 87, respectively.

In some embodiments, the nucleotide sequences encoding the three polypeptide chains are set forth in SEQ ID NO: 80, SEQ ID NO: 86, and SEQ ID NO: 88, respectively.

In one example, the multispecific antibody consists of three polypeptide chains, and the amino acid sequences of the three polypeptide chains are set forth in SEQ ID NO: 81, SEQ ID NO: 85, and SEQ ID NO: 87, respectively.

In some embodiments, the nucleotide sequences encoding the three polypeptide chains are set forth in SEQ ID NO: 82, SEQ ID NO: 86, and SEQ ID NO: 88, respectively.

In one example, the multispecific antibody consists of three polypeptide chains, and the amino acid sequences of the three polypeptide chains are set forth in SEQ ID NO: 83, SEQ ID NO: 85, and SEQ ID NO: 87, respectively.

In some embodiments, the nucleotide sequences encoding the three polypeptide chains are set forth in SEQ ID NO: 84, SEQ ID NO: 86, and SEQ ID NO: 88, respectively.

### ADC

In some specific embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the multispecific antibody comprises a first antigen-binding portion, a second antigen-binding portion, and a third antigen-binding portion, wherein the first antigen-binding portion comprises the CDR1 set forth in SEQ ID NO: 7, the CDR2 set forth in SEQ ID NO: 8, and the CDR3 set forth in SEQ ID NO: 9; the second antigen-binding portion comprises an HCDR1 having the amino acid sequence set forth in SEQ ID NO: 63, an HCDR2 having the amino acid sequence set forth in SEQ ID NO: 64, an HCDR3 having the amino acid sequence set forth in SEQ ID NO: 65, an LCDR1 having the amino acid sequence set forth in SEQ ID NO: 66, an LCDR2 having the amino acid sequence set forth in SEQ ID NO: 67, and an LCDR3 having the amino acid sequence set forth in SEQ ID NO: 68; and the third antigen-binding portion comprises the CDR1 set forth in SEQ ID NO: 4, the CDR2 set forth in SEQ ID NO: 5, and the CDR3 set forth in SEQ ID NO: 6, wherein X₁ is T.

In some specific embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the multispecific antibody comprises a first antigen-binding portion, a second antigen-binding portion, and a third antigen-binding portion, wherein the first antigen-binding portion comprises a CDR1, a CDR2, and a CDR3 of a single variable domain having the amino acid sequence set forth in SEQ ID NO: 36; the second antigen-binding portion comprises an HCDR1, an HCDR2, and an HCDR3 in a heavy chain variable region having the amino acid sequence set forth in SEQ ID NO: 69, and an LCDR1, an LCDR2, and an LCDR3 in a light chain variable region having the amino acid sequence set forth in SEQ ID NO: 70; and the third antigen-binding portion comprises a CDR1, a CDR2, and a CDR3 of a single variable domain having the amino acid sequence set forth in SEQ ID NO: 35. The CDRs are defined, for example, according to Kabat, IMGT, Chothia, Contact, AbM, and/or CCG.

In some specific embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the multispecific antibody comprises a first antigen-binding portion, a second antigen-binding portion, and a third antigen-binding portion, wherein the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 36; the second antigen-binding portion comprises a heavy chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 69, and a light chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 70; and the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 35.

In some specific embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the multispecific antibody comprises a first antigen-binding portion, a second antigen-binding portion, and a third antigen-binding portion, wherein the first antigen-binding portion comprises the amino acid sequence set forth in SEQ ID NO: 36, the second antigen-binding portion comprises a heavy chain variable region having the amino acid sequence set forth in SEQ ID NO: 69 and a light chain variable region having the amino acid sequence set forth in SEQ ID NO: 70, and the third antigen-binding portion comprises the amino acid sequence set forth in SEQ ID NO: 35.

In some specific embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 77, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

In some specific embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the multispecific antibody consists of three polypeptide chains, wherein one polypeptide chain comprises the amino acid sequence set forth in SEQ ID NO: 77, another polypeptide chain comprises the amino acid sequence set forth in SEQ ID NO: 85, and the other polypeptide chain comprises the amino acid sequence set forth in SEQ ID NO: 87.

In some specific embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the multispecific antibody consists of three polypeptide chains, the amino acid sequences of which are set forth in SEQ ID NO: 77, SEQ ID NO: 85, and SEQ ID NO: 87, respectively.

In some embodiments, the multispecific antibody may be modifiable, for example, comprising one or more amino acid insertions, deletions, or substitutions. Herein, the modified multispecific antibody still retains the activity of specifically binding to EGFR (e.g., human EGFR and/or monkey EGFR) and c-Met (e.g., human c-Met and/or monkey c-Met). In some embodiments, the C-terminal lysine of the heavy chain constant region of the multispecific antibody may be present or deleted.

In the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the cytotoxic drug is conjugated to the multispecific antibody via a linker. In some specific embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, each cytotoxic drug is conjugated to the multispecific antibody via one linker. The linker of the present disclosure may be linked to the multispecific antibody by any method known in the art. In some preferred embodiments, the linker is linked to the multispecific antibody via sulfydryl and/or amino. In some more preferred embodiments, the linker is linked to the multispecific antibody via sulfydryl.

In some embodiments, the linker is a cleavable linker or a non-cleavable linker. In some embodiments, the linker is a cleavable linker, which may be, for example, a low pH-dependent degradation type (including a hydrazone bond, a carbonate bond, and the like), a proteolytic type (including a peptide-based bond), a high glutathione concentration-dependent degradation type (including a disulfide bond), or the like. The cleavable linker may be cleaved within the target cell, thereby releasing the cytotoxic drug. In some other embodiments, the linker is a non-cleavable linker, which may be, for example, maleimidocaproyl, or the like.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the multispecific antibody is conjugated to one or more cytotoxic drugs, and the cytotoxic drugs may be, for example, alkaloids, antimetabolites, anti-tumor antibiotics, alkylating agents, platinum-based drugs, or the like. In some embodiments, the cytotoxic drug is a cytotoxic drug acting on DNA. In some embodiments, the cytotoxic drug acting on DNA includes, but is not limited to, calicheamicins, duocarmycins, pyrrolobenzodiazepines (PBDs), topoisomerase I inhibitors, and topoisomerase II inhibitors.

In some embodiments, the cytotoxic drug is a topoisomerase I inhibitor. In some embodiments, the cytotoxic drug is a camptothecin topoisomerase I inhibitor. In some embodiments, the cytotoxic drug is SN-38, an SN-38 derivative, exatecan, or an exatecan derivative. In some specific embodiments, the cytotoxic drug is an exatecan derivative. In some specific embodiments, the cytotoxic drug is an exatecan derivative and has a deuterated modification.

In some embodiments, the cytotoxic drug is linked to the linker via a functional group, and the antibody-drug conjugate is hydrolyzed in tumor cells to form a free cytotoxic drug, thereby exerting an anti-tumor effect.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate is formed by linking a drug-linker having a structure represented by the following formula Ia

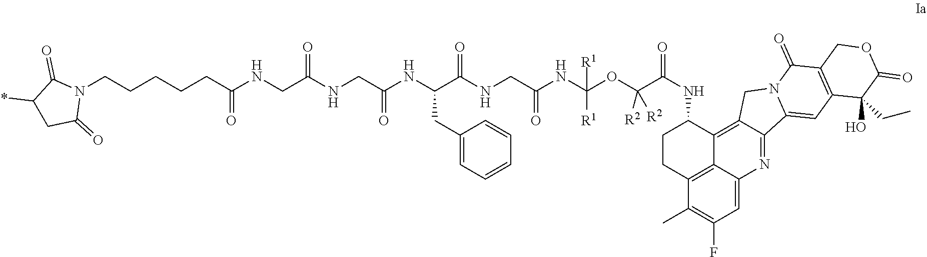

to the multispecific antibody, and the drug-linker is linked to the multispecific antibody at a position indicated by * in the structure represented by formula Ia,
wherein R¹ and R² are each independently selected from a hydrogen atom and a deuterium atom. In some embodiments, the drug-linker is linked to the disulfide bond site of the multispecific antibody at the position indicated by * in the structure represented by formula Ia, preferably linking via a thioether bond.

In some embodiments, R¹ is a hydrogen atom and R² is a deuterium atom.

In some embodiments, the antibody-drug conjugate is formed by linking a drug-linker having a structure represented by the following formula Ia-1

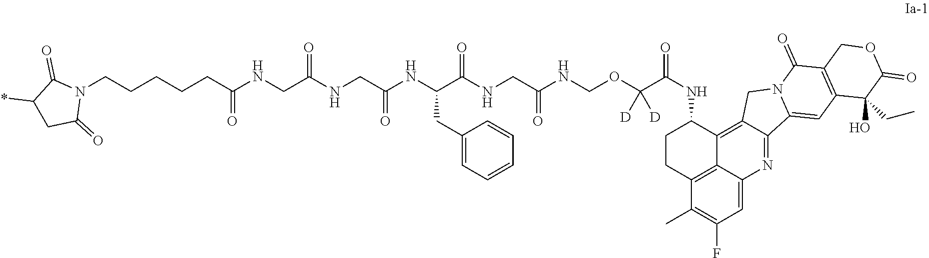 [SMILES: *C1CC(=O)N(CCCCCC(=O)NCC(=O)NCC(=O)N[C@@H](Cc2ccccc2)C(=O)NCC(=O)NCOC([2H])([2H])C(=O)N[C@H]2CCc3c(C)c(F)cc4nc5c(c2c34)Cn2c-5cc3c(c2=O)COC(=O)[C@]3(O)CC)C1=O]

to the multispecific antibody, and the drug-linker is linked to the multispecific antibody at a position indicated by * in the structure represented by formula Ia-1. In some embodiments, the drug-linker is linked to the disulfide bond site of the multispecific antibody at the position indicated by * in the structure represented by formula Ia, preferably linking via a thioether bond.

In the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the number of cytotoxic drugs linked to the multispecific antibody may vary such that the antibody-drug conjugate (ADC) may be heterogeneous, that is, the antibody-drug conjugate comprises multispecific antibodies linked to different numbers of cytotoxic drugs. For example, one multispecific antibody molecule is linked to 0 (i.e., no cytotoxic drug), 1, 2, 3, 4, 5, or 6 or more cytotoxic drug molecules.

By controlling the ratio of the multispecific antibodies linked to different numbers of cytotoxic drugs described above, antibody-drug conjugates having different drug-to-antibody ratios (DARs), stereoisomers thereof, pharmaceutically acceptable salts thereof, or solvates of the antibody-drug conjugates, the stereoisomers thereof, or the pharmaceutically acceptable salts thereof can be produced. It should be understood that the DAR is the average number of linked cytotoxic drugs for each multispecific antibody in the antibody-drug conjugate, the stereoisomer thereof, the pharmaceutically acceptable salt thereof, or the solvate of the antibody-drug conjugate, the stereoisomer thereof, or the pharmaceutically acceptable salt thereof. For example, a “DAR of 6” refers to that the antibody-drug conjugate, the stereoisomer thereof, or the pharmaceutically acceptable salt thereof, or the solvate of the antibody-drug conjugate, the stereoisomer thereof, or the pharmaceutically acceptable salt thereof comprises a heterogeneous mixture in which each multispecific antibody is linked to the same or different numbers of cytotoxic drugs (for example, each multispecific antibody is linked to 0, 1, 2, 3, 4, 5, and/or 6 cytotoxic drugs), but the average number of cytotoxic drugs linked to each multispecific antibody is 6.

In some of the above embodiments, the DAR of the antibody-drug conjugate is 1 to 10, 2 to 8, 2 to 6, 4 to 7, 5 to 6, or 5.5 to 6. In some of the above embodiments, the DAR of the antibody-drug conjugate is 5 to 6. In some of the above embodiments, the DAR of the antibody-drug conjugate is 5.5 to 6. In some of the above embodiments, the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate may also be represented by the structure shown in formula II below:

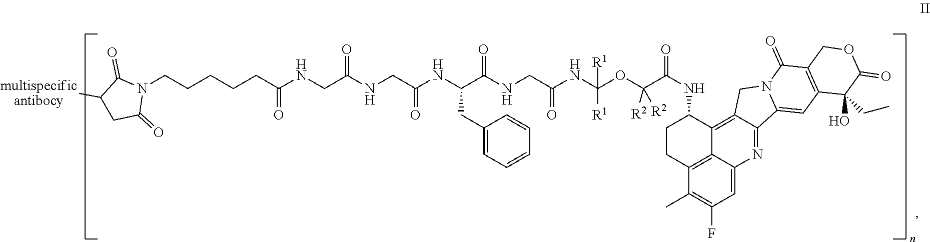

wherein the multispecific antibody is defined as the multispecific antibody of the present disclosure; and
R¹ and R² are each independently selected from a hydrogen atom and a deuterium atom. In some embodiments, R¹ is a hydrogen atom and R² is a deuterium atom.

In some embodiments, position 3 of -(succinimidyl-3-yl-N)- in formula II is linked to the multispecific antibody via a thioether bond. n is synonymous with DAR and represents the average number of cytotoxic drugs linked to each multispecific antibody. In some embodiments, n is 1 to 10, 2 to 8, 2 to 6, 4 to 7, 5 to 6, or 5.5 to 6. In some embodiments, n is 5 to 6. In some embodiments, n is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, the antibody-drug conjugate may be represented by the structure shown in formula II-1 below:

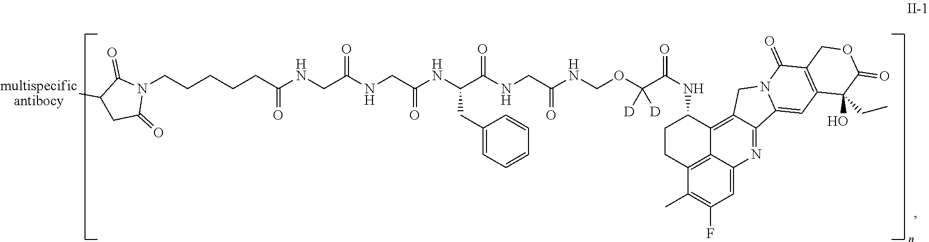

wherein the multispecific antibody is defined as the multispecific antibody of the present disclosure, and n is 1 to 10, 2 to 8, 2 to 6, 4 to 7, 5 to 6, or 5.5 to 6. In some embodiments, n is 3.5 to 5. In some embodiments, n is 5 to 6. In some embodiments, n is 5.5 to 6. In some embodiments, n is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate is formed by linking a drug-linker having a structure represented by formula Ia-1 to the multispecific antibody, and the drug-linker is linked to the multispecific antibody at a position indicated by * in the structure represented by formula Ia-1; and the multispecific antibody comprises a first antigen-binding portion, a second antigen-binding portion, and a third antigen-binding portion, wherein the first antigen-binding portion comprises the CDR1 set forth in SEQ ID NO: 7, the CDR2 set forth in SEQ ID NO: 8, and the CDR3 set forth in SEQ ID NO: 9; the second antigen-binding portion comprises an HCDR1 having the amino acid sequence set forth in SEQ ID NO: 63, an HCDR2 having the amino acid sequence set forth in SEQ ID NO: 64, an HCDR3 having the amino acid sequence set forth in SEQ ID NO: 65, an LCDR1 having the amino acid sequence set forth in SEQ ID NO: 66, an LCDR2 having the amino acid sequence set forth in SEQ ID NO: 67, and an LCDR3 having the amino acid sequence set forth in SEQ ID NO: 68; and the third antigen-binding portion comprises the CDR1 set forth in SEQ ID NO: 4, the CDR2 set forth in SEQ ID NO: 5, and the CDR3 set forth in SEQ ID NO: 6, wherein X₁ is T. In some such embodiments, the DAR of the antibody-drug conjugate is 1 to 10, 2 to 8, 2 to 6, 4 to 7, 5 to 6, or 5.5 to 6; preferably, the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate is formed by linking a drug-linker having a structure represented by formula Ia-1 to the multispecific antibody, and the drug-linker is linked to the multispecific antibody at a position indicated by * in the structure represented by formula Ia-1; and the multispecific antibody comprises a first antigen-binding portion, a second antigen-binding portion, and a third antigen-binding portion, wherein the first antigen-binding portion comprises a CDR1, a CDR2, and a CDR3 of a single variable domain having the amino acid sequence set forth in SEQ ID NO: 36; the second antigen-binding portion comprises an HCDR1, an HCDR2, and an HCDR3 in a heavy chain variable region having the amino acid sequence set forth in SEQ ID NO: 69, and an LCDR1, an LCDR2, and an LCDR3 in a light chain variable region having the amino acid sequence set forth in SEQ ID NO: 70; and the third antigen-binding portion comprises a CDR1, a CDR2, and a CDR3 of a single variable domain having the amino acid sequence set forth in SEQ ID NO: 35. The CDRs are defined, for example, according to Kabat, IMGT, Chothia, Contact, AbM, and/or CCG. In some such embodiments, the DAR of the antibody-drug conjugate is 1 to 10, 2 to 8, 2 to 6, 4 to 7, 5 to 6, or 5.5 to 6; preferably, the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate is formed by linking a drug-linker having a structure represented by formula Ia-1 to the multispecific antibody, and the drug-linker is linked to the multispecific antibody at a position indicated by * in the structure represented by formula Ia-1; and the multispecific antibody comprises a first antigen-binding portion, a second antigen-binding portion, and a third antigen-binding portion, wherein the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 36; the second antigen-binding portion comprises a heavy chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 69, and a light chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 70; and the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 35. In some such embodiments, the DAR of the antibody-drug conjugate is 1 to 10, 2 to 8, 2 to 6, 4 to 7, 5 to 6, or 5.5 to 6; preferably, the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate is formed by linking a drug-linker having a structure represented by formula Ia-1 to the multispecific antibody, and the drug-linker is linked to the multispecific antibody at a position indicated by * in the structure represented by formula Ia-1; and the multispecific antibody comprises a first antigen-binding portion, a second antigen-binding portion, and a third antigen-binding portion, wherein the first antigen-binding portion comprises the amino acid sequence set forth in SEQ ID NO: 36, the second antigen-binding portion comprises a heavy chain variable region having the amino acid sequence set forth in SEQ ID NO: 69 and a light chain variable region having the amino acid sequence set forth in SEQ ID NO: 70, and the third antigen-binding portion comprises the amino acid sequence set forth in SEQ ID NO: 35. In some such embodiments, the DAR of the antibody-drug conjugate is 1 to 10, 2 to 8, 2 to 6, 4 to 7, 5 to 6, or 5.5 to 6; preferably, the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate is formed by linking a drug-linker having a structure represented by formula Ia-1 to the multispecific antibody, and the drug-linker is linked to the multispecific antibody at a position indicated by * in the structure represented by formula Ia-1; and the multispecific antibody consists of three polypeptide chains, wherein one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 77, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87. In some such embodiments, the DAR of the antibody-drug conjugate is 1 to 10, 2 to 8, 2 to 6, 4 to 7, 5 to 6, or 5.5 to 6; preferably, the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate is formed by linking a drug-linker having a structure represented by formula Ia-1 to the multispecific antibody, and the drug-linker is linked to the multispecific antibody at a position indicated by * in the structure represented by formula Ia-1; and the multispecific antibody consists of three polypeptide chains, wherein one polypeptide chain comprises the amino acid sequence set forth in SEQ ID NO: 77, another polypeptide chain comprises the amino acid sequence set forth in SEQ ID NO: 85, and the other polypeptide chain comprises the amino acid sequence set forth in SEQ ID NO: 87. In some such embodiments, the DAR of the antibody-drug conjugate is 1 to 10, 2 to 8, 2 to 6, 4 to 7, 5 to 6, or 5.5 to 6; preferably, the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the antibody-drug conjugate is formed by linking a drug-linker having a structure represented by formula Ia-1 to the multispecific antibody, and the drug-linker is linked to the multispecific antibody at a position indicated by * in the structure represented by formula Ia-1; and the multispecific antibody consists of three polypeptide chains, the amino acid sequences of which are set forth in SEQ ID NO: 77, SEQ ID NO: 85, and SEQ ID NO: 87, respectively. In some such embodiments, the DAR of the antibody-drug conjugate is 1 to 10, 2 to 8, 2 to 6, 4 to 7, 5 to 6, or 5.5 to 6; preferably, the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, in the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, the DAR of the antibody-drug conjugate is 5 to 6, 5.5 to 6, or 6, and the mass fraction of the antibody-drug conjugate in which each multispecific antibody is linked to 6 cytotoxic drugs is not less than 40%, not less than 45%, not less than 50%, not less than 65%, not less than 70%, not less than 75%, not less than 80%, not less than 85%, not less than 90%, not less than 95%, or not less than 99%.

In some embodiments, the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure exhibits one of or a combination of more of the following properties:
(1) binding to human c-Met, preferably binding to human c-Met with a Kp value of 6E-10 M or less;
(2) binding to human EGFR;
(3) binding to monkey c-Met;
(4) binding to monkey EGFR;
(5) internalization in cells expressing c-Met and/or EGFR;
(6) having killing activity against tumor cells expressing c-Met and/or EGFR; and
(7) having a bystander effect.

In some embodiments, the antibody-drug conjugate, the stereoisomer thereof, the pharmaceutically acceptable salt thereof, or the solvate of the antibody-drug conjugate, the stereoisomer thereof, or the pharmaceutically acceptable salt thereof has the properties (1) to (7).

The antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure achieves excellent anti-tumor effect and/or safety. In some embodiments, the antibody-drug conjugate, the stereoisomer thereof, the pharmaceutically acceptable salt thereof, or the solvate of the antibody-drug conjugate, the stereoisomer thereof, or the pharmaceutically acceptable salt thereof exhibits good killing activity against a variety of tumor cells expressing EGFR and/or c-Met. In some embodiments, the antibody-drug conjugate, the stereoisomer thereof, the pharmaceutically acceptable salt thereof, or the solvate of the antibody-drug conjugate, the stereoisomer thereof, or the pharmaceutically acceptable salt thereof has good in vivo anti-tumor activity. In some embodiments, the antibody-drug conjugate, the stereoisomer thereof, the pharmaceutically acceptable salt thereof, or the solvate of the antibody-drug conjugate, the stereoisomer thereof, or the pharmaceutically acceptable salt thereof has excellent safety. In some embodiments, the antibody-drug conjugate, the stereoisomer thereof, the pharmaceutically acceptable salt thereof, or the solvate of the antibody-drug conjugate, the stereoisomer thereof, or the pharmaceutically acceptable salt thereof is less prone to aggregation.

### Pharmaceutical Composition

The present disclosure provides a pharmaceutical composition comprising the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure. In some embodiments, the pharmaceutical composition further comprises pharmaceutically acceptable excipients. The pharmaceutically acceptable excipient includes, for example, an excipient, a diluent, an encapsulating material, a filler, a buffering agent, or other reagents.

### Use

The present disclosure provides use of the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure. The present disclosure further provides use of the pharmaceutical composition of the present disclosure.

In one aspect, the present disclosure provides use of the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure in preparing a medicament for treating a disease expressing c-Met and/or EGFR.

In one aspect, the present disclosure provides use of the pharmaceutical composition of the present disclosure in preparing a medicament for treating a disease expressing c-Met and/or EGFR.

In one aspect, the present disclosure provides use of the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, and one or more additional therapeutic agents in preparing a medicament for treating a disease expressing c-Met and/or EGFR.

In one aspect, the present disclosure provides use of the pharmaceutical composition of the present disclosure and one or more additional therapeutic agents in preparing a medicament for treating a disease expressing c-Met and/or EGFR.

In one aspect, the present disclosure provides a method for treating a disease expressing c-Met and/or EGFR, comprising administering to a subject the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure. In some embodiments, the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure is administered to a subject in a therapeutically effective amount.

In one aspect, the present disclosure provides a method for treating a disease expressing c-Met and/or EGFR, comprising administering to a subject the pharmaceutical composition of the present disclosure. In some embodiments, the pharmaceutical composition of the present disclosure is administered to a subject in a therapeutically effective amount.

In one aspect, the present disclosure provides a method for treating a disease expressing c-Met and/or EGFR, comprising administering to a subject the antibody-drug conjugate, the stereoisomer thereof, or the pharmaceutically acceptable salt thereof, or the solvate of the antibody-drug conjugate, the stereoisomer thereof, or the pharmaceutically acceptable salt thereof of the present disclosure, and one or more additional therapeutic agents. In some embodiments, the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, and one or more additional therapeutic agents are administered to a subject in a therapeutically effective amount.

In one aspect, the present disclosure provides a method for treating a disease expressing c-Met and/or EGFR, comprising administering to a subject the pharmaceutical composition of the present disclosure and one or more additional therapeutic agents. In some embodiments, the pharmaceutical composition of the present disclosure and one or more additional therapeutic agents are administered to a subject in a therapeutically effective amount.

In some embodiments, the disease expressing c-Met and/or EGFR is a tumor. In some embodiments, the additional therapeutic agent may be an anti-tumor therapeutic agent known in the art.

In some embodiments, the method comprises contacting tumor cells with the antibody-drug conjugate, the stereoisomer thereof, the pharmaceutically acceptable salt thereof, or the solvate of the antibody-drug conjugate, the stereoisomer thereof, or the pharmaceutically acceptable salt thereof, or the pharmaceutical composition, thereby killing the tumor cells or inhibiting growth of the tumor cells.

In some embodiments, the method comprises contacting tumor cells with the antibody-drug conjugate, the stereoisomer thereof, the pharmaceutically acceptable salt thereof, or the solvate of the antibody-drug conjugate, the stereoisomer thereof, or the pharmaceutically acceptable salt thereof, or the pharmaceutical composition, and simultaneously or sequentially contacting the tumor cells with one or more additional therapeutic agents, thereby killing the tumor cells or inhibiting growth of the tumor cells.

In some embodiments, administration of the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, or the pharmaceutical composition of the present disclosure to a subject can kill tumor cells or inhibit tumor cell growth. In some embodiments, administration of a therapeutically effective amount of the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, or the pharmaceutical composition of the present disclosure to a subject can kill tumor cells or inhibit tumor cell growth.

In some embodiments, administration of the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, and one or more additional therapeutic agents to a subject can kill tumor cells or inhibit tumor cell growth. In some embodiments, administration of a therapeutically effective amount of the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, and one or more additional therapeutic agents to a subject can kill tumor cells or inhibit tumor cell growth. In some embodiments, administration of the pharmaceutical composition of the present disclosure and one or more additional therapeutic agents to a subject can kill tumor cells or inhibit tumor cell growth. In some embodiments, administration of a therapeutically effective amount of the pharmaceutical composition of the present disclosure and one or more additional therapeutic agents to a subject can kill tumor cells or inhibit tumor cell growth.

In some embodiments, the tumor is a c-Met- and/or EGFR-positive tumor. In some embodiments, the tumor is epithelial cell carcinoma, squamous cell carcinoma, glioblastoma, breast cancer, ovarian cancer, lung cancer, lung adenocarcinoma, colorectal cancer, anal cancer, prostate cancer, kidney cancer, liver cancer, bladder cancer, head and neck cancer, gastric cancer, pancreatic cancer, skin cancer, oral cancer, pharyngeal cancer, nasal cancer, tongue cancer, esophageal cancer, testicular cancer, vaginal cancer, cervical cancer, spleen cancer, testicular cancer, thyroid cancer, salivary gland cancer, and/or thymus cancer. In some embodiments, the lung cancer includes non-small cell lung cancer (NSCLC) and small cell lung cancer (SCLC).

### Kit

The present disclosure provides a kit comprising the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, or the pharmaceutical composition of the present disclosure.

The present disclosure describes a kit comprising the antibody-drug conjugate, the stereoisomer thereof, the pharmaceutically acceptable salt thereof, or the solvate of the antibody-drug conjugate, the stereoisomer thereof, or the pharmaceutically acceptable salt thereof, or the pharmaceutical composition. The kit can be used for implementing use of the antibody-drug conjugate, the stereoisomer thereof, the pharmaceutically acceptable salt thereof, or the solvate of the antibody-drug conjugate, the stereoisomer thereof, or the pharmaceutically acceptable salt thereof provided by the present disclosure, or use of the pharmaceutical composition provided by the present disclosure, or other use. In some embodiments, the kit may comprise the antibody-drug conjugate, the stereoisomer thereof, the pharmaceutically acceptable salt thereof, or the solvate of the antibody-drug conjugate, the stereoisomer thereof, or the pharmaceutically acceptable salt thereof, or the pharmaceutical composition; optionally, the kit may further comprise an instruction for use. The kit may further comprise other materials as required from commercial and user perspectives, for example, other buffers, diluents, needles, syringes, and the like.

### Linker-Payload

In some aspects, the present disclosure provides a linker-payload having a structure represented by formula I below:

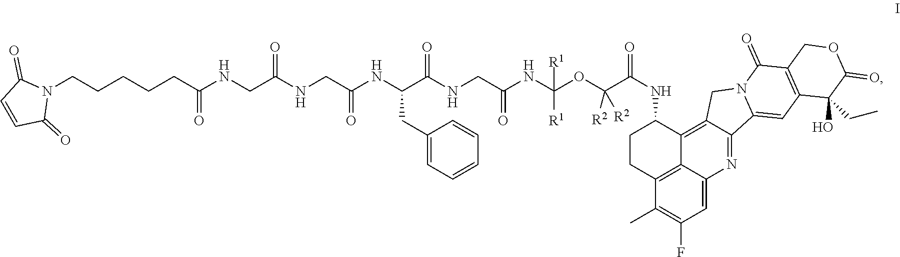

wherein R¹ and R² are each independently selected from a hydrogen atom and a deuterium atom.

In some embodiments, R¹ is a hydrogen atom and R² is a deuterium atom.

In one specific embodiment, the present disclosure provides a linker-payload having a structure represented by formula I-1 below:

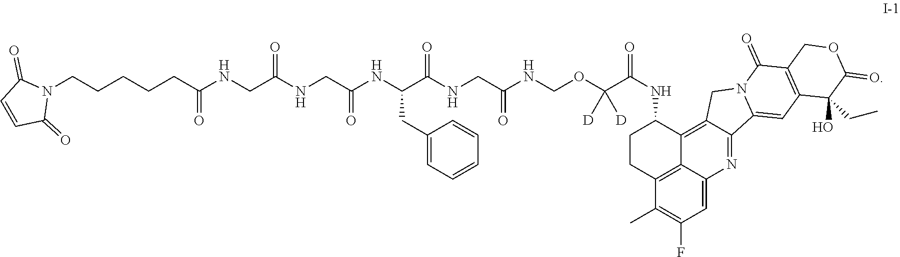 [SMILES: [2H]C([2H])(OCNC(=O)CNC(=O)[C@H](Cc1ccccc1)NC(=O)CNC(=O)CNC(=O)CCCCCN1C(=O)C=CC1=O)C(=O)N[C@H]1CCc2c(C)c(F)cc3nc4c(c1c23)Cn1c-4cc2c(c1=O)COC(=O)[C@]2(O)CC]

### Preparation Method

The present disclosure provides a method for preparing the antibody-drug conjugate, the stereoisomer thereof, the pharmaceutically acceptable salt thereof, or the solvate of the antibody-drug conjugate, the stereoisomer thereof, or the pharmaceutically acceptable salt thereof of the present disclosure, comprising treating the multispecific antibody under a reducing condition, and then reacting the multispecific antibody with the linker-payload selected from the structure represented by formula I, wherein R¹ and R² are each independently selected from a hydrogen atom and a deuterium atom. In some embodiments, R¹ is a hydrogen atom and R² is a deuterium atom.

In some embodiments, the method comprises treating the multispecific antibody under a reducing condition, and then reacting the multispecific antibody with the linker-payload selected from the structure represented by formula I-1.

In some embodiments, the reducing condition is in the presence of TCEP.

### DETAILED DESCRIPTION

The present disclosure further provides the following specific embodiments, which, however, are not intended to limit the scope of the present disclosure:

Embodiment 1. An antibody-drug conjugate, a stereoisomer thereof, a pharmaceutically acceptable salt thereof, or a solvate of the antibody-drug conjugate, the stereoisomer thereof, or the pharmaceutically acceptable salt thereof, comprising a multispecific antibody, wherein the multispecific antibody comprises
(i) a first antigen-binding portion binding to a first antigen;
(ii) a second antigen-binding portion binding to a second antigen; and
(iii) a third antigen-binding portion binding to the first antigen,
wherein the first antigen is c-Met, and the second antigen is EGFR; and the first antigen-binding portion and the third antigen-binding portion are both single variable domains and each independently comprise any one of the following:
(1) a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, X₁ being selected from S and T, preferably T;
(2) a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9;
(3) a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
(4) a CDR1 comprising the amino acid sequence set forth in SEQ ID NO: 10, a CDR2 comprising the amino acid sequence set forth in SEQ ID NO: 11, and a CDR3 comprising the amino acid sequence set forth in SEQ ID NO: 12;
(5) a CDR1 comprising the amino acid sequence set forth in SEQ ID NO: 13, a CDR2 comprising the amino acid sequence set forth in SEQ ID NO: 14, and a CDR3 comprising the amino acid sequence set forth in SEQ ID NO: 15;
(6) a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18;
(7) a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21; or
(8) a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24.

Embodiment 2. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 1, wherein the first antigen-binding portion and the third antigen-binding portion each independently comprise any one of the following:
(1) a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, X₁ being selected from S and T, preferably T;
(2) a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO:

9; or
(8) a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24.

Embodiment 3. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 2, wherein the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; and the third antigen-binding portion comprises: a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6, wherein X₁ is selected from S and T; preferably, X₁ is T.

Embodiment 4. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 2, wherein the first antigen-binding portion comprises: a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9; and the third antigen-binding portion comprises: a CDR1 comprising the amino acid sequence set forth in SEQ ID NO: 22, a CDR2 comprising the amino acid sequence set forth in SEQ ID NO: 23, and a CDR3 comprising the amino acid sequence set forth in SEQ ID NO: 24.

Embodiment 5. An antibody-drug conjugate, a stereoisomer thereof, a pharmaceutically acceptable salt thereof, or a solvate of the antibody-drug conjugate, the stereoisomer thereof, or the pharmaceutically acceptable salt thereof, comprising a multispecific antibody, wherein the multispecific antibody comprises
(i) a first antigen-binding portion binding to a first antigen;
(ii) a second antigen-binding portion binding to a second antigen; and
(iii) a third antigen-binding portion binding to the first antigen, wherein the first antigen is c-Met, and the second antigen is EGFR; and the first antigen-binding portion and the third antigen-binding portion are both single variable domains and each independently comprise a CDR1, a CDR2, and a CDR3 of a single variable domain having the amino acid sequence set forth in SEQ ID NO: 35, 36, 26, 27, 25, 28, 29, 30, 31, 32, 33, 34, 37, 38, or 39.

Embodiment 6. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 5, wherein the first antigen-binding portion and the third antigen-binding portion each independently comprise a CDR1, a CDR2, and a CDR3 of a single variable domain having the amino acid sequence set forth in SEQ ID NO: 35, 36, or 38.

Embodiment 7. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 6, wherein the first antigen-binding portion comprises a CDR1, a CDR2, and a CDR3 of a single variable domain having the amino acid sequence set forth in SEQ ID NO: 36, and the third antigen-binding portion comprises a CDR1, a CDR2, and a CDR3 of a single variable domain having the amino acid sequence set forth in SEQ ID NO: 35.

Embodiment 8. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 6, wherein the first antigen-binding portion comprises a CDR1, a CDR2, and a CDR3 of a single variable domain having the amino acid sequence set forth in SEQ ID NO: 36, and the third antigen-binding portion comprises a CDR1, a CDR2, and a CDR3 of a single variable domain having the amino acid sequence set forth in SEQ ID NO: 38.

Embodiment 9. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 8, wherein the first antigen-binding portion and the third antigen-binding portion each independently comprise an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 40, 41, 35, 36, 26, 27, 25, 28, 29, 30, 31, 32, 33, 34, 37, 38, 39, or 42. Embodiment 10. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 9, wherein the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 41.

Embodiment 11. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 10, wherein the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 27, 36, or 37; preferably, the first antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 36.

Embodiment 12. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 11, wherein the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 40.

Embodiment 13. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 12, wherein the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 26, 33, 34, or 35; preferably, the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 35.

Embodiment 14. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 11, wherein the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 42. Embodiment 15. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 14, wherein the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 32, 38, or 39; preferably, the third antigen-binding portion comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 38.

Embodiment 16. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 8, wherein the first antigen-binding portion and the third antigen-binding portion are selected from any one of the following:
(1) the first antigen-binding portion comprising the amino acid sequence set forth in SEQ ID NO: 36, and the third antigen-binding portion comprising the amino acid sequence set forth in SEQ ID NO: 35;
(2) the first antigen-binding portion comprising the amino acid sequence set forth in SEQ ID NO: 27, and the third antigen-binding portion comprising the amino acid sequence set forth in SEQ ID NO: 26;
(3) the first antigen-binding portion comprising the amino acid sequence set forth in SEQ ID NO: 36, and the third antigen-binding portion comprising the amino acid sequence set forth in SEQ ID NO: 33;
(4) the first antigen-binding portion comprising the amino acid sequence set forth in SEQ ID NO: 36, and the third antigen-binding portion comprising the amino acid sequence set forth in SEQ ID NO: 34;
(5) the first antigen-binding portion comprising the amino acid sequence set forth in SEQ ID NO: 36, and the third antigen-binding portion comprising the amino acid sequence set forth in SEQ ID NO: 32;
(6) the third antigen-binding portion comprising the amino acid sequence set forth in SEQ ID NO: 36, and the first antigen-binding portion comprising the amino acid sequence set forth in SEQ ID NO: 38; or
(7) the third antigen-binding portion comprising the amino acid sequence set forth in SEQ ID NO: 36, and the first antigen-binding portion comprising the amino acid sequence set forth in SEQ ID NO: 39.

Embodiment 17. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 16, wherein the single variable domain is derived from a camelid or is humanized.

Embodiment 18. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 17, wherein the second antigen-binding portion is a Fab, an scFv, or an scFab.

Embodiment 19. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 18, wherein the second antigen-binding portion comprises: an HCDR1 comprising the amino acid sequence set forth in SEQ ID NO: 63, an HCDR2 comprising the amino acid sequence set forth in SEQ ID NO: 64, an HCDR3 comprising the amino acid sequence set forth in SEQ ID NO: 65, an LCDR1 comprising the amino acid sequence set forth in SEQ ID NO: 66, an LCDR2 comprising the amino acid sequence set forth in SEQ ID NO: 67, and an LCDR3 comprising the amino acid sequence set forth in SEQ ID NO: 68.

Embodiment 20. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 18, wherein the second antigen-binding portion comprises an HCDR1, an HCDR2, and an HCDR3 in a heavy chain variable region having the amino acid sequence set forth in SEQ ID NO: 69, and an LCDR1, an LCDR2, and an LCDR3 in a light chain variable region having the amino acid sequence set forth in SEQ ID NO: 70.

Embodiment 21. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 20, wherein the second antigen-binding portion comprises a heavy chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 69, and a light chain variable region having an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 70.

Embodiment 22. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 21, wherein the heavy chain variable region of the second antigen-binding portion comprises the amino acid sequence set forth in SEQ ID NO: 69, and the light chain variable region of the second antigen-binding portion comprises the amino acid sequence set forth in SEQ ID NO: 70.

Embodiment 23. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 22, wherein the second antigen-binding portion is murine, chimeric, or humanized.

Embodiment 24. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 23, wherein the third antigen-binding portion and the first antigen-binding portion are fused to each other, optionally via a peptide linker.

Embodiment 25. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 24, wherein the third antigen-binding portion, at the C-terminus, is fused to the N-terminus of the first antigen-binding portion.

Embodiment 26. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 25, wherein the multispecific antibody further comprises
(iv) an Fc domain composed of two Fc polypeptides.

Embodiment 27. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 26, wherein the first antigen-binding portion, at the C-terminus, is fused to the N-terminus of one of the Fc polypeptides of the Fc domain, the second antigen-binding portion is a Fab, and the second antigen-binding portion, at the C-terminus of the Fab heavy chain, is fused to the N-terminus of the other Fc polypeptide of the Fc domain.

Embodiment 28. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 26 or 27, wherein the Fc domain is an IgG Fc domain, preferably an IgG1 Fc domain.

Embodiment 29. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 28, wherein the IgG Fc domain is a human IgG Fc domain, preferably a human IgG1 Fc domain.

Embodiment 30. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 26 to 29, wherein the Fc domain comprises an amino acid substitution that promotes association of the two Fc polypeptides of the Fc domain.

Embodiment 31. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 30, wherein according to the EU numbering, one of the Fc polypeptides of the Fc domain comprises amino acid substitutions 354C and 366Y/W, and the other Fc polypeptide comprises amino acid substitutions 349C, 366S, 368A, and 407T/V.

Embodiment 32. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 26 to 31, wherein the Fc domain comprises an amino acid substitution that reduces or eliminates binding of a CH3 region of one Fc polypeptide in the Fc domain to Protein A.

Embodiment 33. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 32, wherein according to the EU numbering, the Fc domain comprises amino acid substitutions (a) 435R or (b) 435R and 436F occurring in only one of the Fc polypeptides.

Embodiment 34. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 26 to 29, wherein according to the EU numbering, one of the Fc polypeptides of the Fc domain comprises amino acid substitutions: 349C, 366S, 368A, 407V, 435R, and 436F, and the other Fc polypeptide comprises amino acid substitutions: 354C and 366W.

Embodiment 35. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 26 to 29, wherein according to the EU numbering, one of the Fc polypeptides of the Fc domain comprises amino acid substitutions: 349C, 366S, 368A, 407V, and 435R, and the other Fc polypeptide comprises amino acid substitutions: 354C and 366W.

Embodiment 36. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 35, wherein the multispecific antibody is trivalent.

Embodiment 37. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 36, wherein the multispecific antibody consists of three polypeptide chains, wherein:
(1) one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 77, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87;
(2) one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 71, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87;
(3) one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 73, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87;
(4) one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 75, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87;
(5) one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 79, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87;
(6) one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 81, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87; or
(7) one polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 83, another polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 85, and the other polypeptide chain comprises an amino acid sequence having at least 85%, 86%, 87%, 88%, 89%, 90%, 91%, 92%, 93%, 94%, 95%, 96%, 97%, 98%, 99%, or 100% identity to the amino acid sequence set forth in SEQ ID NO: 87.

Embodiment 38. An antibody-drug conjugate, a stereoisomer thereof, a pharmaceutically acceptable salt thereof, or a solvate of the antibody-drug conjugate, the stereoisomer thereof, or the pharmaceutically acceptable salt thereof, comprising a multispecific antibody, and the multispecific antibody consists of three polypeptide chains, wherein:

(1) one polypeptide chain comprises the amino acid sequence set forth in SEQ ID NO: 77, another polypeptide chain comprises the amino acid sequence set forth in SEQ ID NO: 85, and the other polypeptide chain comprises the amino acid sequence set forth in SEQ ID NO: 87;
(2) one polypeptide chain comprises the amino acid sequence set forth in SEQ ID NO: 71, another polypeptide chain comprises the amino acid sequence set forth in SEQ ID NO: 85, and the other polypeptide chain comprises the amino acid sequence set forth in SEQ ID NO: 87;
(3) one polypeptide chain comprises the amino acid sequence set forth in SEQ ID NO: 73, another polypeptide chain comprises the amino acid sequence set forth in SEQ ID NO: 85, and the other polypeptide chain comprises the amino acid sequence set forth in SEQ ID NO: 87;
(4) one polypeptide chain comprises the amino acid sequence set forth in SEQ ID NO: 75, another polypeptide chain comprises the amino acid sequence set forth in SEQ ID NO: 85, and the other polypeptide chain comprises the amino acid sequence set forth in SEQ ID NO: 87;
(5) one polypeptide chain comprises the amino acid sequence set forth in SEQ ID NO: 79, another polypeptide chain comprises the amino acid sequence set forth in SEQ ID NO: 85, and the other polypeptide chain comprises the amino acid sequence set forth in SEQ ID NO: 87;
(6) one polypeptide chain comprises the amino acid sequence set forth in SEQ ID NO: 81, another polypeptide chain comprises the amino acid sequence set forth in SEQ ID NO: 85, and the other polypeptide chain comprises the amino acid sequence set forth in SEQ ID NO: 87; or
(7) one polypeptide chain comprises the amino acid sequence set forth in SEQ ID NO: 83, another polypeptide chain comprises the amino acid sequence set forth in SEQ ID NO: 85, and the other polypeptide chain comprises the amino acid sequence set forth in SEQ ID NO: 87.

Embodiment 39. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 38, the multispecific antibody is afucosylated.

Embodiment 40. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 39, wherein the antibody-drug conjugate is formed by linking a drug-linker having a structure represented by the following formula Ia

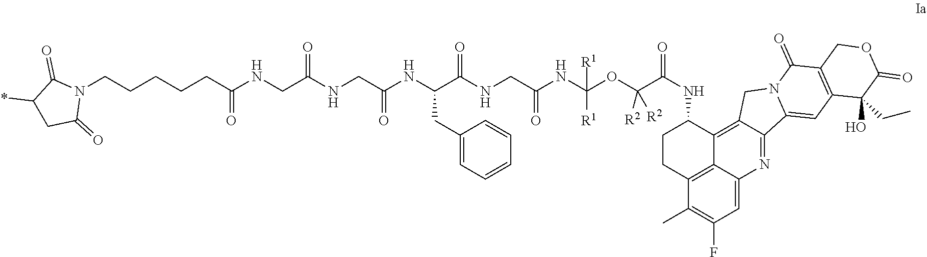

to the multispecific antibody, and the drug-linker is linked to the multispecific antibody at a position indicated by * in the structure represented by formula Ia,
wherein
R¹ and R² are each independently selected from a hydrogen atom and a deuterium atom.

Embodiment 41. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to Embodiment 40, wherein R¹ is a hydrogen atom, and R² is a deuterium atom.

Embodiment 42. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 41, wherein the DAR of the antibody-drug conjugate is 1 to 10, 2 to 8, 2 to 6, 4 to 7, 5 to 6, or 5.5 to 6.

Embodiment 43. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 41, wherein the DAR of the antibody-drug conjugate is 2, 3, 4, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

Embodiment 44. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 43, wherein the antibody-drug conjugate, the stereoisomer thereof, the pharmaceutically acceptable salt thereof, or the solvate of the antibody-drug conjugate, the stereoisomer thereof, or the pharmaceutically acceptable salt thereof exhibits one of or a combination of more of the following properties:
(1) binding to human c-Met, preferably binding to human c-Met with a Kp value of 6E-10 M or less;
(2) binding to human EGFR;
(3) binding to monkey c-Met;
(4) binding to monkey EGFR;
(5) internalization in cells expressing c-Met and/or EGFR;
(6) having killing activity against tumor cells expressing c-Met and/or EGFR; and
(7) having a bystander effect.

Embodiment 45. A pharmaceutical composition, comprising the antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 44; optionally, the pharmaceutical composition further comprises a pharmaceutically acceptable excipient.

Embodiment 46. A method for preparing the antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 40 to 44, comprising treating the multispecific antibody under a reducing condition, and then reacting the multispecific antibody with a linker-payload selected from a structure represented by formula I, wherein the structure represented by formula I is as follows:

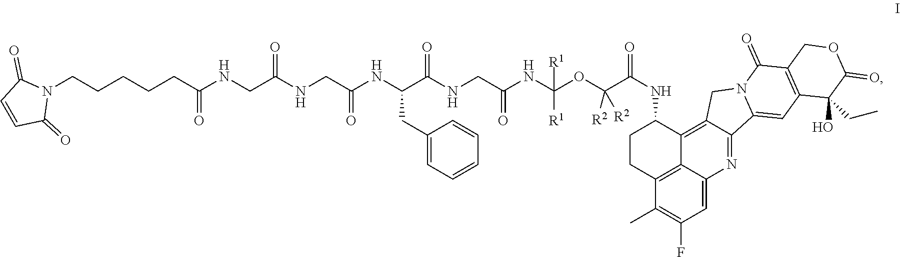

wherein R¹ and R² are each independently selected from a hydrogen atom and a deuterium atom.

Embodiment 47. The method according to Embodiment 46, wherein R¹ is a hydrogen atom and R² is a deuterium atom.

Embodiment 48. Use of the antibody-drug conjugate, the stereoisomer thereof, or the pharmaceutically acceptable salt thereof, or the solvate of the antibody-drug conjugate, the stereoisomer thereof, or the pharmaceutically acceptable salt thereof according to any one of Embodiments 1 to 44, or the pharmaceutical composition according to Embodiment 45 in preparing a medicament for treating a disease expressing c-Met and/or EGFR.

Embodiment 49. Use of the antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 44, or the pharmaceutical composition according to Embodiment 45, and one or more additional therapeutic agents in preparing a medicament for treating a disease expressing c-Met and/or EGFR.

Embodiment 50. The use according to Embodiment 48 or 49, wherein the disease expressing c-Met and/or EGFR is a tumor.

Embodiment 51. The use according to Embodiment 49 or 50, wherein the additional therapeutic agent is an anti-tumor therapeutic agent.

Embodiment 52. The use according to Embodiment 50 or 51, wherein the tumor is a c-Met- and/or EGFR-positive tumor; preferably, the tumor is epithelial cell carcinoma, squamous cell carcinoma, glioblastoma, breast cancer, ovarian cancer, lung cancer, lung adenocarcinoma, colorectal cancer, anal cancer, prostate cancer, kidney cancer, liver cancer, bladder cancer, head and neck cancer, gastric cancer, pancreatic cancer, skin cancer, oral cancer, pharyngeal cancer, nasal cancer, tongue cancer, esophageal cancer, testicular cancer, vaginal cancer, cervical cancer, spleen cancer, testicular cancer, thyroid cancer, salivary gland cancer, and/or thymus cancer.

Embodiment 53. A method for treating a disease expressing c-Met and/or EGFR, comprising administering to a subject the antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 44, or the pharmaceutical composition according to Embodiment 45.

Embodiment 54. The method according to Embodiment 53, wherein the disease expressing c-Met and/or EGFR is a tumor.

Embodiment 55. The method according to Embodiment 54, wherein the method comprises contacting tumor cells with the antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 44, or the pharmaceutical composition according to Embodiment 45, thereby killing the tumor cells or inhibiting growth of the tumor cells.

Embodiment 56. The method according to any one of Embodiments 53 to 55, wherein the method further comprises administering to the subject one or more additional therapeutic agents.

Embodiment 57. The method according to Embodiment 56, wherein the additional therapeutic agent is an anti-tumor therapeutic agent.

Embodiment 58. The method according to any one of Embodiments 54 to 57, wherein the tumor is a c-Met- and/or EGFR-positive tumor; preferably, the tumor is epithelial cell carcinoma, squamous cell carcinoma, glioblastoma, breast cancer, ovarian cancer, lung cancer, lung adenocarcinoma, colorectal cancer, anal cancer, prostate cancer, kidney cancer, liver cancer, bladder cancer, head and neck cancer, gastric cancer, pancreatic cancer, skin cancer, oral cancer, pharyngeal cancer, nasal cancer, tongue cancer, esophageal cancer, testicular cancer, vaginal cancer, cervical cancer, spleen cancer, testicular cancer, thyroid cancer, salivary gland cancer, and/or thymus cancer.

Embodiment 59. A kit, comprising the antibody-drug conjugate, the stereoisomer thereof, the pharmaceutically acceptable salt thereof, or the solvate of the antibody-drug conjugate, the stereoisomer thereof, or the pharmaceutically acceptable salt thereof according to any one of Embodiments 1 to 44, or the pharmaceutical composition according to Embodiment 45; optionally, the kit further comprises an instruction for use.

For clarity, the present disclosure is further described with the following examples, which are, however, not intended to limit the scope of the present disclosure. The reagents used in the present disclosure are commercially available and can be used without further purification. The amivantamab used in the examples was purchased from Janssen, with NDC No. 57894-501-01. RAA22/B09-57 used in the examples was prepared according to conventional methods for antibody production. Vectors were constructed first, followed by transfection into eukaryotic cells, purification, and expression. The sequences are derived from SEQ ID NO: 59 (anti-EGFR-HC), SEQ ID NO: 61 (anti-EGFR-LC), SEQ ID NO: 60 (anti-c-Met-HC), and SEQ ID NO: 62 (anti-c-Met-LC) in the patent publication US20230183358A1.

### Example 1. Construction of Anti-Human c-Met V_{H}H Phage Display Library

A recombinant human c-Met-Fc fusion protein (SinoBiological, Cat. No. 10692-H02H) was mixed and emulsified with complete Freund's adjuvant in a volume ratio of 1:1. A bactrian camel was first immunized by subcutaneous multipoint injection, and then boosted with the recombinant human c-Met-Fc fusion protein mixed and emulsified with incomplete Freund's adjuvant in a volume ratio of 1:1 at intervals of 2 weeks. The serum was collected after the fourth or fifth immunization and assayed for anti-human c-Met antibody titer. After multiple rounds of immunization, the peripheral blood of the bactrian camel was collected, and peripheral blood mononuclear cells (PBMCs) were isolated. The total RNA of the PBMCs was extracted and then reversely transcribed into cDNA, and the variable region (V_{H}H) sequence of the camel antibody was amplified by nested PCR.

The V_{H}H coding fragment obtained from the amplification was digested with PstI/NotI endonuclease and inserted into a phagemid vector pMECS (NTCC Plasmid Vector, Strain, Cell and Gene Collection, Cat. No. pMECS) to construct a recombinant vector, which was then transferred into Escherichia coli TG1 (Lucigen, Cat. No. 60502-1) by electroporation to construct a library stock. The library stock was amplified to logarithmic growth phase. Library amplification was performed by adding M13KO7 helper phages (New England Biolabs, Cat. No. N0315S), with shaking overnight at 200 rpm at 28 °C. The bacterial liquid was centrifuged, and the supernatant was collected. ¼ supernatant volume of a PEG6000/NaCl solution (20% PEG6000 (w/v), 2.5 M NaCl) was added. The mixture was incubated on ice for 1-2 h to settle the phages and centrifuged. The settled phages were collected and resuspended in PBS, and then 20% glycerol was added. The suspension was preserved at −80 °C as a V_{H}H phage display library.

### Example 2. Anti-Human c-Met V_{H}H Screening

The V_{H}H phage display library was subjected to solid-phase panning, and the single clones obtained from the panning were cultured. Expression was induced by isopropyl-β-D-thiogalactoside (IPTG), and the supernatant was prepared.

The picked clones were subjected to positive identification by indirect ELISA for human c-Met-His (SinoBiological, Cat. No. 10692-H08H). Positive clones that bind only to human c-Met-His with relatively high signal values were selected for preservation and sequenced. Positive clones 1B-1B2, 1B-3B11, 1B-1C7, 1B-1B6, 1B-1A8, 3B-1C7, 4&5B-2F01, and 4&5C-12B04 were selected via the screening. As determined by sequence analysis, the amino acid sequence of the V_{H}H of 1B-3B11 is set forth in SEQ ID NO: 25, the amino acid sequence of the V_{H}H of 1B-1B6 is set forth in SEQ ID NO: 26, the amino acid sequence of the V_{H}H of 1B-1C7 is set forth in SEQ ID NO: 27, the amino acid sequence of the V_{H}H of 1B-1A8 is set forth in SEQ ID NO: 28, and the amino acid sequence of the V_{H}H of 1B-1B2 is set forth in SEQ ID NO: 29; the amino acid sequence of the V_{H}H of 3B-1C7 is set forth in SEQ ID NO: 30; the amino acid sequence of the V_{H}H of 4&5B-2F01 is set forth in SEQ ID NO: 31; the amino acid sequence of the V_{H}H of 4&5C-12B04 is set forth in SEQ ID NO: 32.

### Example 3. Preparation of Anti-Human c-Met V_{H}H-Fc Chimeric Antibodies

The V_{H}H sequences of the positive clones obtained from the screening were linked to a human Fc region to construct V_{H}H-Fc chimeric antibodies. Specifically, the V_{H}H sequences sequenced in Example 2 were inserted into a pcDNA3.1 (+) eukaryotic expression vector containing a human IgG1 Fc region. These V_{H}H-Fc chimeric antibodies were expressed using an Expifectamine™ CHO Transfection Kit transient transfection and expression system (Thermo Fisher Scientific Inc., Cat. No. A29129). The sequences of VL1016-069 and VH1016-069 in Patent No. US20200079872A1 were separately inserted into a pcDNA3.1 (+) eukaryotic expression vector containing a human IgG1 constant region (the amino acid sequence is SEQ ID NO: 92), and a chimeric antibody 1016-069 was expressed as a control using the same method.

As determined by sequence analysis, the amino acid sequence of 1B-3B11-Fc is set forth in SEQ ID NO: 43, the amino acid sequence of 1B-1B6-Fc is set forth in SEQ ID NO: 44, the amino acid sequence of 1B-1C7-Fc is set forth in SEQ ID NO: 45, the amino acid sequence of 1B-1A8-Fc is set forth in SEQ ID NO: 46, the amino acid sequence of 1B-1B2-Fc is set forth in SEQ ID NO: 47, the amino acid sequence of 3B-1C7-Fc is set forth in SEQ ID NO: 48, the amino acid sequence of chimeric antibody 4&5B-2F01-Fc is set forth in SEQ ID NO: 49, and the amino acid sequence of chimeric antibody 4&5C-12B04-Fc is set forth in SEQ ID NO: 50.

### Example 4. Affinity of Anti-Human V_{H}H-Fc Chimeric Antibodies for Human and Cynomolgus c-Met

4.1 Determination of Affinity of Antibodies for Human and Cynomolgus c-Met by Surface Plasmon Resonance

The anti-human c-Met V_{H}H-Fc chimeric antibodies were tested for affinity using a biomolecular interaction analysis system (GE, Biacore T200 or Biacore 8K). An anti-hIgG (Fc) antibody (GE, Cat. No. BR-1008-39) was amino-coupled to a CM5 sensor chip. The anti-human c-Met V_{H}H-Fc chimeric antibodies were diluted to 1 μg/mL with a running buffer (137 mM NaCl, 2.7 mM KCl, 10 mM Na₂HPO₄·12H₂O, 1.8 mM KH₂PO₄, 0.05% surfactant P-20 (w/v), pH 7.4), and the capture was conducted by passing the dilutions through experimental channels at a flow rate of 30 μL/min. Human c-Met-His (SinoBiological, Cat. No. 10692-H08H) or cynomolgus c-Met-His (SinoBiological, Cat. No. 90304-C08H) was diluted with the running buffer to 100 nM, 50 nM, 25 nM, 12.5 nM, 6.25 nM, and 3.125 nM, and the association was conducted for 200 s by passing the dilutions at a flow rate of 50 μL/min. Then dissociation was conducted for 600-1400 s by stopping sample injection. The data were analyzed with software and fitted using a Langmuir 1:1 model. The association rate constant kₐ (1/Ms), the dissociation rate constant k_{d} (1/s), and the equilibrium dissociation constant K_{D} (M) were calculated. The results are shown in Tables 1-1 and 1-2. 1B-1B2-Fc, 1B-3B11-Fc, 1B-1C7-Fc, 1B-1B6-Fc, 1B-1A8-Fc, 3B-1C7-Fc, 4&5B-2F01-Fc, and 4&5C-12B04-Fc all exhibited high affinity for human c-Met protein and cross-reactions with cynomolgus c-Met protein.

TABLE 1-1
Affinity of V_{H}H-Fc chimeric antibodies for human c-Met and cynomolgus c-Met

| V_{H}H-Fc | | | | | | |
|---|---|---|---|---|---|---|
| Chimeric | Human c-Met-His | | | Cynomolgus c-Met-His | | |
| antibody | kₐ (1/Ms) | k_{d} (1/s) | K_{D} (M) | kₐ (1/Ms) | k_{d} (1/s) | K_{D} (M) |
| 1B-3B11-Fc | 5.03E+04 | 6.81E−05 | 1.35E−09 | 4.35E+04 | 2.88E−05 | 6.64E−10 |
| 1B-1B2-Fc | 1.41E+05 | 5.60E−04 | 3.99E−09 | 7.84E+04 | 2.15E−04 | 2.74E−09 |
| 1B-1C7-Fc | 2.21E+05 | 4.43E−05 | 2.00E−10 | 2.12E+05 | 3.99E−05 | 1.88E−10 |
| 1B-1B6-Fc | 8.44E+04 | 6.96E−04 | 8.25E−09 | 5.07E+04 | 3.47E−04 | 6.85E−09 |
| 1B-1A8-Fc | 1.73E+05 | 1.12E−04 | 6.48E−10 | 1.30E+05 | 8.80E−05 | 6.77E−10 |
| 3B-1C7-Fc | 1.76E+05 | 5.56E−05 | 3.17E−10 | 2.89E+05 | 7.35E−05 | 2.54E−10 |

TABLE 1-2
Affinity of V_{H}H-Fc chimeric antibodies for human c-Met and cynomolgus c-Met

| V_{H}H-Fc | | | | | | |
|---|---|---|---|---|---|---|
| Chimeric | Human c-Met-His | | | Cynomolgus c-Met-His | | |
| antibody | kₐ (1/Ms) | k_{d} (1/s) | K_{D} (M) | kₐ (1/Ms) | k_{d} (1/s) | K_{D} (M) |
| 4&5B-2F01-Fc | 9.51E+04 | 6.44E−05 | 6.76E−10 | 5.21E+04 | 5.44E−04 | 1.04E−08 |
| 4&5C-12B04-Fc | 1.74E+05 | 8.96E−05 | 5.16E−10 | 1.45E+05 | 7.22E−05 | 4.98E−10 |
| 1016-069 | 2.83E+05 | 2.52E−04 | 8.91E−10 | 4.30E+05 | 1.97E−04 | 4.59E−10 |

### 4.2 Assay of Binding of Antibodies to Cells by Flow Cytometry

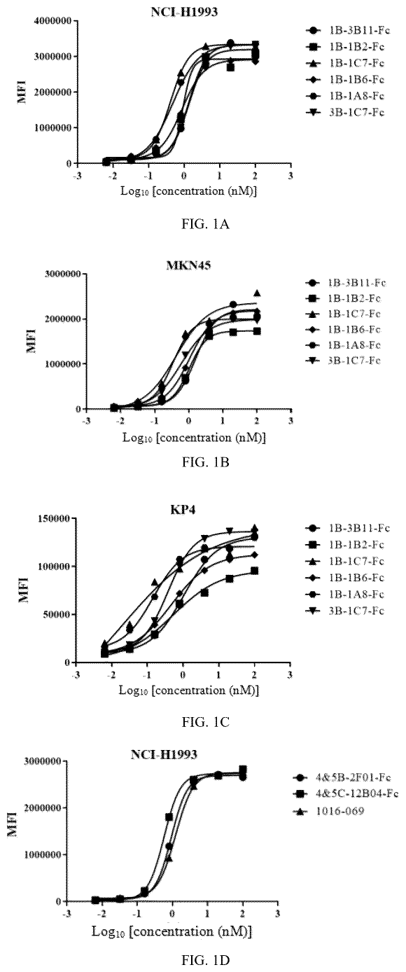

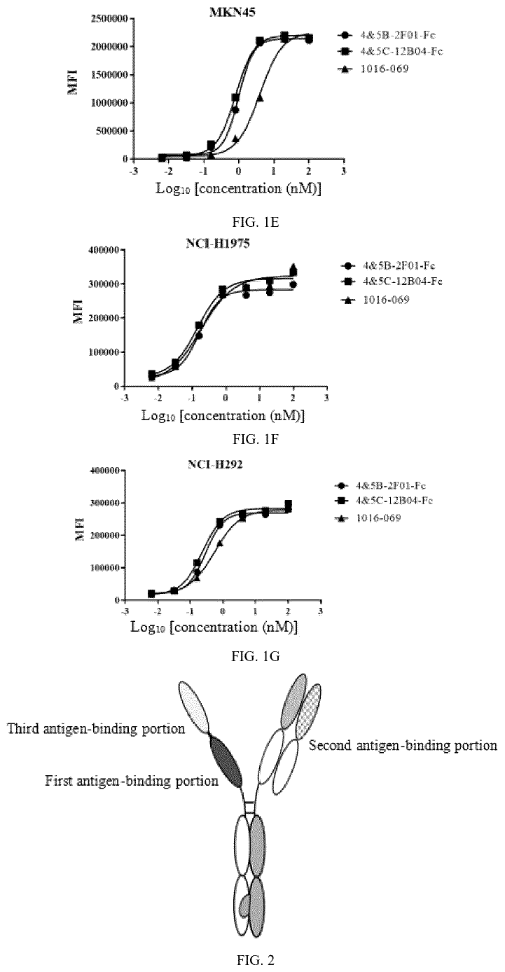

The binding of the anti-human c-Met V_{H}H-Fc chimeric antibodies to target cells with different c-Met expression levels was determined by flow cytometry. The NCI-H1993 cells (BeNa Culture Collection, Cat. No. BNCC342186) are human lung adenocarcinoma cells with a high c-Met expression level, the MKN45 cells (Nanjing Cobioer Biosciences Co., Ltd., Cat. No. CBP60488) are human gastric cancer cells with a medium c-Met expression level, the KP4 cells (Nanjing Cobioer Biosciences Co., Ltd., Cat. No. CBP60541) are human pancreatic cancer cells with a low c-Met expression level, the NCI-H1975 cells (BeNa Culture Collection, Cat. No. BNCC100690) are human non-small cell lung adenocarcinoma cells with a low to medium c-Met expression level, and the NCI-H292 cells (BeNa Culture Collection, Cat. No. BNCC100671) are human epidermal lung cancer cells with a low to medium c-Met expression level. After 2×10⁵ target cells were incubated with serially diluted anti-human c-Met V_{H}H-Fc chimeric antibodies (serially diluted 5-fold from the initial concentration of 100 nM to yield 7 concentrations) on ice for 1 h, the cells were washed. A PE-labeled anti-human IgG Fc antibody (Jackson Immuno Research, Cat. No. 109-116-170) was added, and the mixture was incubated on ice for 0.5 h. The cells were washed and then tested using a flow cytometer (Thermo Fisher Scientific Inc., Attune NXT). The results are shown in FIGS. 1A to 1G and Tables 2-1 and 2-2. 1B-1B2-Fc, 1B-3B11-Fc, 1B-1C7-Fc, 1B-1B6-Fc, 1B-1A8-Fc, 3B-1C7-Fc, 4&5B-2F01-Fc, and 4&5C-12B04-Fc all exhibited high binding ability to target cells with different c-Met expression levels, among which 4&5B-2F01-Fc and 4&5C-12B04-Fc were superior to the control 1016-069.

TABLE 2-1
Binding of anti-human c-Met V_{H}H-Fc
chimeric antibodies to target cells

| V_{H}H-FC | NCI-H1993 | MKN45 | |
|---|---|---|---|
| Chimeric | cell | cell | KP4 cell |
| antibody | EC₅₀ (nM) | EC₅₀ (nM) | EC₅₀ (nM) |
| 1B-3B11-Fc | 1.333 | 1.615 | 1.049 |
| 1B-1B2-Fc | 0.910 | 1.034 | 0.526 |
| 1B-1C7-Fc | 0.407 | 0.421 | 0.019 |
| 1B-1B6-Fc | 0.854 | 1.056 | 0.471 |
| 1B-1A8-Fc | 0.477 | 0.345 | 0.143 |
| 3B-1C7-Fc | 1.352 | 0.592 | 0.375 |

TABLE 2-2
Binding of anti-human c-Met V_{H}H-Fc chimeric antibodies to target cells

| VHH-Fc | NCI-H1993 | MKN45 | NCI-H1975 | NCI-H292 |
|---|---|---|---|---|
| Chimeric | cell | cell | cell | cell |
| antibody | EC₅₀ (nM) | EC₅₀ (nM) | EC₅₀ (nM) | EC₅₀ (nM) |
| 4&5B-2F01-Fc | 0.924 | 0.988 | 0.166 | 0.292 |
| 4&5C-12B04-Fc | 0.574 | 0.809 | 0.144 | 0.236 |
| 1016-069 | 1.217 | 3.950 | 0.185 | 0.550 |

### Example 5. Epitope Difference Analysis Between Different Clones of Anti-Human c-Met V_{H}H

Epitope competition relationship analysis was performed using a biomolecular interaction system (Fortebio, Cat. No. Octet RED96). An Anti-Penta-HIS (HIS1K) sensor (Fortebio, Cat. No. 18-5120) was used, and the c-Met-His protein (SinoBiological, Cat. No. 10692-H08H) was diluted to about 5 μg/mL using a running buffer. The sensor was immersed in the diluted antigen sample. The immobilization height was controlled to about 1 nm by adjusting the binding time. Then the antigen sample interacted with a first antibody A and a second antibody B in sequence, and the B antibody binding signal was detected to determine whether the two antibodies recognize the same epitope. The results are shown in Tables 3-1, 3-2, and 3-3. The determination criteria were: a value >60% indicated that the 2 antibodies do not compete at all; a value between 20% and 60% indicated a partial competition of the 2 antibodies (there may be a crossover at the epitopes); a value <20% indicated a complete competition of the 2 antibodies. If the self-reaction signal (underlined) was <20%, the data were determined valid

As can be seen from the data in Table 3-1, the two antibodies 1B-3B11-Fc and 1B-1A8-Fc exhibited a complete competition; 1B-3B11-Fc and 1B-1A8-Fc demonstrated no significant competitive relationship with the other three candidate antibodies, suggesting different epitopes. Thus, the antibodies 1B-3B11-Fc and 1B-1B2-Fc, 1B-3B11-Fc and 1B-1C7-Fc, 1B-3B11-Fc and 1B-1B6-Fc, 1B-1A8-Fc and 1B-1B2-Fc, 1B-1A8-Fc and 1B-1C7-Fc, 1B-1A8-Fc and 1B-1B6-Fc, 1B-1B2-Fc and 1B-1C7-Fc, 1B-1B2-Fc and 1B-1B6-Fc, and 1B-1C7-Fc and 1B-1B6-Fc can simultaneously bind to different epitopes of the c-Met antigen. As can be seen from the data in Table 3-2, the two antibodies 3B-1C7-Fc and 1B-1B2-Fc exhibited a complete competition. Thus, by combining the data from Table 3-1, it can be seen that: the antibodies 1B-1A8-Fc and 3B-1C7-Fc, 3B-1C7-Fc and 1B-1C7-Fc, and 3B-1C7-Fc and 1B-1B6-Fc can simultaneously bind to different epitopes of the c-Met antigen. As can be seen from the data in Table 3-3, 4&5B-2F01-Fc and 4&5C-12B04-Fc exhibited a complete competition with 1016-069.

TABLE 3-1
Epitope difference analysis

| | 1B- | 1B- | 1B- | 1B- | 1B- |
|---|---|---|---|---|---|
| AB# | 3B11-Fc | 1B2-Fc | 1C7-Fc | 1B6-Fc | 1A8-Fc |
| 1B-3B11-Fc | 9.83% | 132.28% | 118.99% | 126.72% | 8.16% |
| 1B-1B2-Fc | 120.47% | 7.44% | 120.97% | 122.01% | 117.16% |
| 1B-1C7-Fc | 91.78% | 87.01% | 0.00% | 97.85% | 91.06% |
| 1B-1B6-Fc | 125.54% | 119.93% | 118.44% | 13.35% | 125.90% |
| 1B-1A8-Fc | 20.31% | 122.78% | 116.44% | 122.38% | 17.76% |

TABLE 3-2
Epitope difference analysis

| | 1B- | 1B- | 1B- | 1B- | 3B- |
|---|---|---|---|---|---|
| AB# | 3B11-Fc | 1B2-Fc | 1C7-Fc | 1B6-Fc | 1C7-Fc |
| 1B-3B11-Fc | 8.85% | 100.96% | 94.39% | 136.09% | 135.70% |
| 1B-1B2-Fc | 126.48% | 0.12% | 91.08% | 120.10% | 7.11% |
| 1B-1C7-Fc | 126.48% | 95.31% | 7.51% | 126.45% | 122.76% |
| 1B-1B6-Fc | 133.73% | 122.60% | 121.83% | 5.41% | 117.22% |
| 3B-1C7-Fc | 137.10% | 12.03% | 141.34% | 161.78% | 13.40% |

TABLE 3-3
Epitope difference analysis

| AB# | 4&5B-2F01-Fc | 4&5C-12B04-Fc | 1016-069 |
|---|---|---|---|
| 4&5B-2F01-Fc | −4.7% | −3.6% | 0.8% |
| 4&5C-12B04-Fc | 4.4% | −1.0% | 3.0% |
| 1016-069 | −1.9% | 1.5% | 1.2% |

### Example 6. Construction, Expression, Purification, and Affinity Assay of Anti-Human c-Met V H H-Fc Humanized Antibodies

The anti-human c-Met V_{H}H-Fc chimeric antibodies 1B-1B6-Fc, 1B-1C7-Fc, and 4&5C-12B04-Fc were subjected to humanization. The humanized V_{H}H sequences were inserted into a pcDNA3.1 (+) eukaryotic expression vector containing a human IgG1 constant region. These V_{H}H-Fc humanized antibodies were expressed using the Expifectamine™ CHO Transfection Kit transient transfection and expression system (Thermo Fisher Scientific Inc., Cat. No. A29129). Among them, the humanization of chimeric antibody 1B-1B6-Fc gave three humanized antibodies 1B-1B6-V1, 1B-1B6-V2, and 1B-1B6-V3 with full-length amino acid sequences set forth in SEQ ID NOs: 51, 53, and 55, respectively, and full-length nucleotide sequences set forth in SEQ ID NOs: 52, 54, and 56, respectively; the humanization of chimeric antibody 1B-1C7-Fc gave two humanized antibodies 1B-1C7-V1 and 1B-1C7-V2 with full-length amino acid sequences set forth in SEQ ID NOs: 57 and 59, respectively, and full-length nucleotide sequences set forth in SEQ ID NOs: 58 and 60, respectively; the humanization of chimeric antibody 4&5C-12B04-Fc gave two humanized V_{H}Hs: 12B04-V1 and 12B04-V2, with amino acid sequences set forth in SEQ ID NOs: 38 and 39, respectively.

Through the surface plasmon resonance technology, the affinity of the acquired humanized antibodies and chimeric antibodies for human c-Met protein was detected on a biomolecular interaction analysis system (GE, Biacore 8K). An anti-hIgG (Fc) antibody (GE, Cat. No. BR-1008-39) was amino-coupled to a CM5 sensor chip. The anti-human c-Met V_{H}H-Fc chimeric antibodies were diluted to 2 μg/mL with a running buffer (137 mM NaCl, 2.7 mM KCl, 10 mM Na₂HPO₄·12H₂O, 1.8 mM KH₂PO₄, 0.05% surfactant P-20 (w/v), pH 7.4), and the capture was conducted for 90 s by passing the dilutions through experimental channels at a flow rate of 30 μL/min. Human c-Met-His protein (SinoBiological, Cat. No. 10692-H08H) was diluted with the running buffer to 100 nM, 50 nM, 25 nM, 12.5 nM, 6.25 nM, and 3.125 nM, and the association was conducted at a flow rate of 50 μL/min. The association curve was recorded.

The affinity data of the humanized antibodies (1B-1B6-V1, 1B-1B6-V2, 1B-1B6-V3, 1B-1C7-V1, and 1B-1C7-V2) and the chimeric antibodies (1B-1B6-Fc and 1B-1C7-Fc) for human c-Met protein are shown in Table 4.

TABLE 4
Binding affinity of anti-human c-Met V_{H}H-Fc chimeric antibodies
and humanized antibodies for human c-Met-His

| | kₐ (1/Ms) | k_{d} (1/s) | K_{D} (M) |
|---|---|---|---|
| 1B-1C7-Fc | 1.39E+05 | 3.11E−05 | 2.24E−10 |
| 1B-1C7-V1 | 3.68E+05 | 1.30E−02 | 3.54E−08 |
| 1B-1C7-V2 | 3.36E+05 | 1.52E−03 | 4.51E−09 |
| 1B-1B6-Fc | 6.72E+04 | 7.91E−04 | 1.18E−08 |
| 1B-1B6-V1 | 4.83E+05 | 1.11E−02 | 2.31E−08 |
| 1B-1B6-V2 | 1.18E+05 | 2.05E−03 | 1.74E−08 |
| 1B-1B6-V3 | 1.11E+05 | 2.03E−03 | 1.82E−08 |

The assay demonstrated that 1B-1B6-Fc and its humanized antibodies and 1B-1C7-Fc and its humanized antibodies can specifically bind to c-Met-His protein.

### Example 7. Construction, Expression, and Purification of Anti-EGFR/Anti-c-Met Multispecific Antibodies

The anti-EGFR/anti-c-Met multispecific antibody was prepared by connecting 2 humanized V_{H}H molecules targeting c-Met in series. The multispecific antibody Fc domain was subjected to knob-into-hole engineering (according to the EU numbering, mutations Y349C, T366S, L368A, and Y407V were introduced into one of the Fc polypeptides, and mutations S354C and T366W were introduced into the other Fc polypeptide), and a mutation that hinders the binding to Protein A was further introduced into one of the Fc polypeptides of the Fc domain (according to the EU numbering, H435R and Y436F, or H435R).

The anti-EGFR/anti-c-Met multispecific antibodies were constructed according to the configuration shown in FIG. 2, and designated as V12, V41, V35, V42, V67, V73, and V74, respectively, wherein the second antigen-binding portion was an antigen-binding domain binding to EGFR in a Fab form. Specifically, the anti-EGFR/anti-c-Met multispecific antibody had three polypeptide chains. One polypeptide chain (designated as anti-c-Met-V_{H}H-Fc) contained two antigen-binding domains binding to c-Met connected in series (derived from chimeric antibodies 1B-1B6-Fc, 1B-1C7-Fc, and 4&5C-12B04-Fc, and their humanized antibodies, as shown in Table 5), and for V12, V41, V35, V42, V67, V73, and V74, this polypeptide chain had the amino acid sequences set forth in SEQ ID NOs: 71, 73, 75, 77, 79, 81, and 83, respectively, and the nucleotide sequences set forth in SEQ ID NOs: 72, 74, 76, 78, 80, 82, and 84, respectively. The other two polypeptide chains formed an antigen-binding domain binding to EGFR in a Fab form (the variable region sequence binding to EGFR is derived from Patent No. CN100497389C), in which one polypeptide chain was designated as anti-EGFR-HC-Fc (with its amino acid sequence set forth in SEQ ID NO: 85 and nucleotide sequence set forth in SEQ ID NO: 86), and the other polypeptide chain was designated as anti-EGFR-LC (with its amino acid sequence set forth in SEQ ID NO: 87 and nucleotide sequence set forth in SEQ ID NO: 88). The nucleotide sequences encoding anti-c-Met-V_{H}H-Fc, anti-EGFR-HC-Fc, and anti-EGFR-LC were separately inserted into pcDNA3.1 (+) eukaryotic expression vectors to give expression vectors expressing the corresponding polypeptide chains. The expression vectors described above were co-transfected into FUT8 gene-knockout CHO-S cells (designated as CHO FUT8-cells) in a transfection ratio of vector anti-c-Met-V_{H}H-Fc: vector anti-EGFR-HC-Fc: vector anti-EGFR-LC=1.5:1:1.5 using a CHOgro® high-yield expression system (Cat. No. MIR 6270), with a transfected cell density of 4×10⁶ cells/mL. The culture was continued until day 10 after transfection, and the cell culture supernatant was collected by centrifugation. The protein purification was performed by Protein A affinity chromatography, CHT chromatography, and gel chromatography using the ÄKTA pure protein purification system (GE Healthcare). The protein concentration was measured using a UV-Vis spectrophotometer (NanoDrop One C, Thermo Scientific). Electrophoresis (reducing SDS-PAGE), molecular weight (TOF MS), and sequence analysis confirmed that multispecific antibodies with expected structures and sequences were obtained.

TABLE 5
Sources of components of antigen-binding domains binding to
c-Met in anti-EGFR/anti-c-Met multispecific antibodies

| | Third antigen-binding | First antigen-binding |
|---|---|---|
| Name | portion | portion |
| V12 | 1B-1B6 | 1B-1C7 |
| V41 | 1B-1B6-V1 | 1B-1C7-V1 |
| V35 | 1B-1B6-V2 | 1B-1C7-V1 |
| V42 | 1B-1B6-V3 | 1B-1C7-V1 |
| V67 | 4&5C-12B04 | 1B-1C7-V1 |
| V73 | 12B04-V1 | 1B-1C7-V1 |
| V74 | 12B04-V2 | 1B-1C7-V1 |

### Example 8. Affinity of Anti-EGFR/Anti-c-Met Multispecific Antibodies for Human c-Met

Referring to the method in Example 6, the binding of chimeric multispecific antibodies V12 and V67 and humanized multispecific antibodies V35, V41, V42, V73, and V74 to human c-Met protein was detected by surface plasmon resonance. The tests showed that as shown in Table 6, all the antibodies can specifically bind to human c-Met-His protein, and the affinity of the humanized multispecific antibodies is comparable to that of the chimeric multispecific antibodies.

TABLE 6
Binding affinity of anti-EGFR/anti-c-Met
multispecific antibodies for human c-Met-His

| | kₐ (1/Ms) | k_{d} (1/s) | K_{D} (M) |
|---|---|---|---|
| V12 | 1.42E+05 | 4.62E−05 | 3.25E−10 |
| V41 | 9.70E+04 | 3.57E−05 | 3.68E−10 |
| V35 | 1.05E+05 | 3.34E−05 | 3.18E−10 |
| V42 | 1.00E+05 | 4.39E−05 | 4.38E−10 |
| V67 | 2.54E+05 | 7.01E−05 | 2.76E−10 |
| V73 | 1.97E+05 | 9.03E−05 | 4.58E−10 |
| V74 | 2.34E+05 | 1.28E−04 | 5.48E−10 |

### Example 9. Binding of Anti-EGFR/Anti-c-Met Multispecific Antibodies to Tumor Cells Expressing EGFR and c-Met

The binding of the anti-EGFR/anti-c-Met multispecific antibodies (including amivantamab, V42, and V73) to A431 cells expressing EGFR and c-Met (high EGFR expression and low c-Met expression, from Center of Excellence in Molecular Cell Science, CAS), NCI-H1975 cells (medium EGFR expression and low to medium c-Met expression, from BeNa Culture Collection), and NCI-H1993 cells (medium EGFR expression and high c-Met expression, from BeNa Culture Collection) was analyzed by flow cytometry, with an hIgG1 (Biointron, Cat. No. B117901) as the negative control.

A431 cells, NCI-H1975 cells, and NCI-H1993 cells in the logarithmic growth phase were adjusted to a viable cell density of 5×10⁶ to 1×10⁷ cells/mL using an RPMI medium (Hyclon, Cat. No. SH30809.01) containing 2% of fetal bovine serum, and seeded in 96-well U-bottom cell culture plates (Costar, Cat. No. 3799) at 50 μL/well. Different concentrations of the anti-EGFR/anti-c-Met multispecific antibodies were prepared in the medium described above, with a highest concentration of 500 nM followed by 5-fold serial dilutions to yield a total of 10 concentration levels. The antibodies at different concentrations were added to the 96-well cell culture plate described above at 50 μL/well. The mixture was mixed thoroughly and incubated at 4 °C for 1 h. The cells were washed with a pre-cooled running buffer (MACS, Cat. No. 130-091-221), and the supernatant was discarded. A pre-cooled fluorescent labeled goat anti-human IgG antibody (Jackson, Cat. No. 109-116-170) was added, and the cells were resuspended at 100 μL/well, mixed thoroughly, and incubated at 4 °C for 30 min. After washing, the pre-cooled running buffer was added at 40 μL/well to resuspend the cells. The mixture was mixed thoroughly, and the cells were collected while flowing on the iQue3 flow cytometer. The data were analyzed using software.

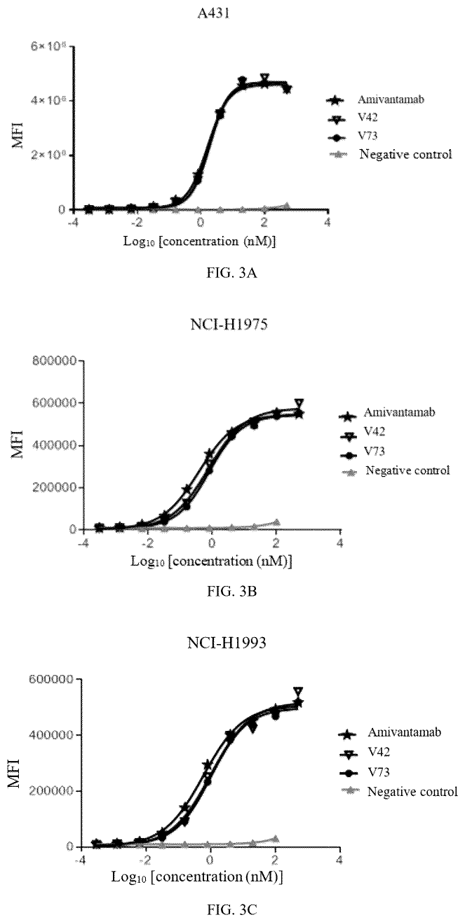

Table 7 and FIGS. 3A to 3C show the binding ability of the anti-EGFR/anti-c-Met multispecific antibodies to A431 cells, NCI-H1975 cells, and NCI-H1993 cells. The results demonstrated that the binding ability of V42 and V73 to the three cell lines was substantially comparable to that of amivantamab.

TABLE 7
The binding EC₅₀ values and maximum binding amounts of the anti-EGFR/
anti-c-Met multispecific antibodies to EGFR and c-Met on the surface of tumor cells

| | A431 | | NCI-H1975 | | NCI-H1993 | |
|---|---|---|---|---|---|---|
| | EC₅₀ (nM) | Top (MFI) | EC₅₀ (nM) | Top (MFI) | EC₅₀ (nM) | Top (MFI) |
| V42 | 1.852 | 4423651 | 0.755 | 600865 | 1.029 | 557298 |
| V73 | 1.905 | 4467149 | 0.7941 | 554727 | 1.018 | 520132 |
| Amivantamab | 1.62 | 4461530 | 0.3776 | 550125 | 0.5957 | 518725 |
| Negative control | / | 168103 | / | 38741 | / | 31204 |

“/” denotes that the curve could not give a value by fitting.

### Example 10. Inhibition of HGF-Stimulated c-Met Phosphorylation and Downstream Signaling Pathways by Anti-EGFR/Anti-c-Met Multispecific Antibodies

The inhibition of HGF stimulation-induced c-Met phosphorylation and downstream signaling pathways by anti-EGFR/anti-c-Met multispecific antibodies (including amivantamab, V42, and V73) was analyzed by Western Blotting, with an hIgG1 (Biointron, Cat. No. B117901) as the negative control.

The coding sequences of HGF-a (with its amino acid sequence set forth in SEQ ID NO: 61) and HGF-β (with its amino acid sequence set forth in SEQ ID NO: 62) were separately inserted into pcDNA3.1 (+) eukaryotic expression vectors, expressed using the Expifectamine™ CHO Transfection Kit transient transfection and expression system (Thermo Fisher Scientific Inc., Cat. No. A29129), and purified using the Ni Sepharose excel nickel column (GE, Cat. No. 17-3712-01) to give HGF.

A549 cells (medium EGFR expression and medium c-Met expression, non-small cell lung cancer, from Cell Resource Center, Institute of Basic Medical Sciences, Chinese Academy of Medical Sciences) in the logarithmic growth phase were collected, counted, and then adjusted to a viable cell density of 4×10⁵ cells/mL using an RPMI medium (Hyclon, Cat. No. SH30809.01) containing 10% of fetal bovine serum. The cells were added to 6-well cell culture plates at 2 mL/well. The plates were incubated in a cell incubator at 37 °C with 5% CO₂ for 6 h, and then the medium was discarded. The RPMI medium was added at 1 mL/well, and the cells were starved overnight. The 6-well cell culture plates after overnight starvation were taken out, and the original medium was discarded. The anti-EGFR/anti-c-Met multispecific antibodies at a final concentration of 100 nM, or both HGF at a final concentration of 100 ng/ml and the anti-EGFR/anti-c-Met multispecific antibodies at a final concentration of 100 nM, were added. The plates were incubated in a cell incubator at 37 °C with 5% CO₂ for 15 min. The cell culture plates were placed on ice and washed with pre-cooled PBS. A lysis buffer containing a protease inhibitor and a phosphatase inhibitor was added for lysis for 30 min. The protein was collected and quantified by the BCA method. The c-Met phosphorylation and downstream signaling pathways were detected by Western Blotting.

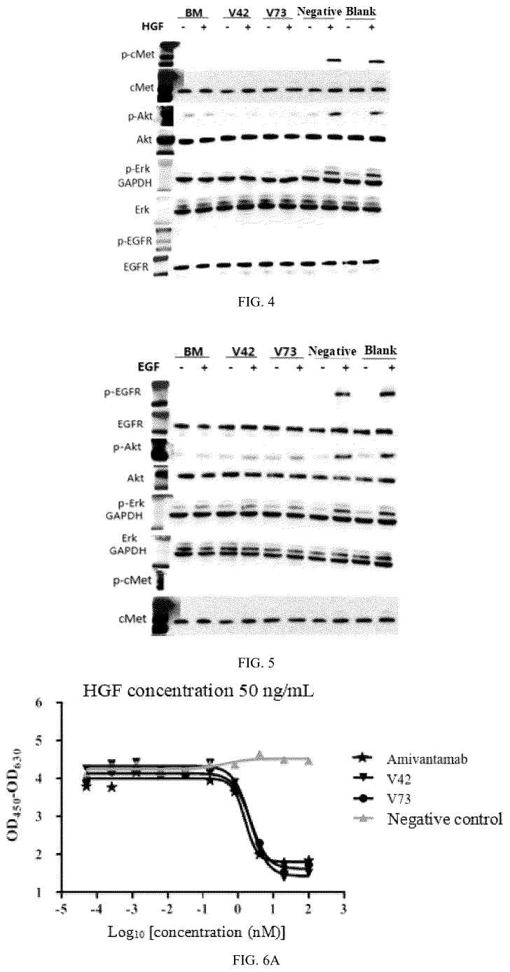

FIG. 4 shows the inhibitory effects of anti-EGFR/anti-c-Met multispecific antibodies on c-Met phosphorylation and downstream signaling pathways. The results demonstrated that the inhibitory effects of V42 and V73 on HGF-stimulated c-Met phosphorylation and downstream signaling pathways were comparable to those of amivantamab (BM in FIG. 4).

### Example 11. Inhibition of EGF-Stimulated EGFR Phosphorylation and Downstream Signaling Pathways by Anti-EGFR/Anti-c-Met Multispecific Antibodies

The inhibition of EGF stimulation-induced EGFR phosphorylation and downstream signaling pathways by anti-EGFR/anti-c-Met multispecific antibodies (including amivantamab, V42, and V73) was analyzed by Western Blotting, with an hIgG1 (Biointron, Cat. No. B117901) as the negative control.

A549 cells in the logarithmic growth phase were collected, counted, and then adjusted to a viable cell density of 4×10⁵ cells/mL using a complete medium. The cells were added to 6-well cell culture plates at 2 mL/well. The plates were incubated in a cell incubator at 37 °C with 5% CO₂ for 6 h, and then the complete medium was discarded. An RPMI medium (Hyclon, Cat. No. SH30809.01) was added at 1 mL/well, and the cells were starved overnight. The 6-well cell culture plates after overnight starvation were taken out, and the original medium was discarded. The anti-EGFR/anti-c-Met multispecific antibodies at a final concentration of 200 nM, or both EGF (R&D, Cat. No. 236-EG) at a final concentration of 40 ng/ml and the anti-EGFR/anti-c-Met multispecific antibodies at a final concentration of 200 nM, were added. The plates were incubated in a cell incubator at 37° C. with 5% CO₂ for 15 min. The cell culture plates were placed on ice and washed with pre-cooled PBS. A lysis buffer containing a protease inhibitor and a phosphatase inhibitor was added for lysis for 30 min. The protein was collected and quantified by the BCA method. The EGFR phosphorylation and downstream signaling pathways were detected by Western Blotting.

FIG. 5 shows the inhibitory effects of anti-EGFR/anti-c-Met multispecific antibodies on EGFR phosphorylation and downstream signaling pathways. The results demonstrated that the inhibitory effects of V42 and V73 on EGF-stimulated EGFR phosphorylation and downstream signaling pathways were comparable to those of amivantamab (BM in FIG. 5).

### Example 12. Activity of Anti-EGFR/Anti-c-Met Multispecific Antibodies in Competing with HGF Ligand for Binding to c-Met

The activity of the anti-EGFR/anti-c-Met multispecific antibodies (including amivantamab, V42, and V73) in competing with the HGF ligand for binding to c-Met was analyzed by ELISA, with an hIgG1 (Biointron, Cat. No. B117901) as the negative control.

2.0 mg of biotin (Thermo, Cat. No. 20217) was dissolved in 590 μL of DMSO (Sigma, Cat. No. D2650). The mixture was mixed thoroughly. The biotin solution was added in a ratio of 2.7 μL of the biotin solution per 100 μL of HGF protein (prepared according to Example 10, at a concentration of 2 mg/mL). The mixture was mixed and incubated at room temperature for 40 min to prepare biotin-labeled HGF. A high-adsorption 96-well plate coated with c-Met-His (Corning, Cat. No. 9018) incubated overnight at 4 °C was taken out, and the solution in the plate was discarded. The plate was washed 3 times with PBST, blocked with 3% BSA at room temperature for 2 h, and then washed 3 times with PBST. An equal-volume mixture solution of biotin-labeled HGF and anti-EGFR/anti-c-Met multispecific antibodies (the final concentration of the anti-EGFR/anti-c-Met multispecific antibodies was 100 nM, and the final concentration of the HGF protein was 50 ng/mL, 10 ng/ml, or 1 ng/ml) was added, and the mixture was incubated at room temperature for 2 h. The plate was washed 3 times with PBST before an HRP-Avidin antibody (Invitrogen, Cat. No. 18-4100-51) was added. The plate was then incubated at room temperature for 1 h. The plate was washed 5 times with PBST before TMB (Thermo, 00-4201-56) was added. The mixture was incubated at room temperature in the dark for 10 min. A 1 M sulfuric acid stop solution was added, and the mixture was left to stand at room temperature for 5 min. The absorbance values at 450 nm were read on a microplate reader with 630 nm as the reference wavelength.

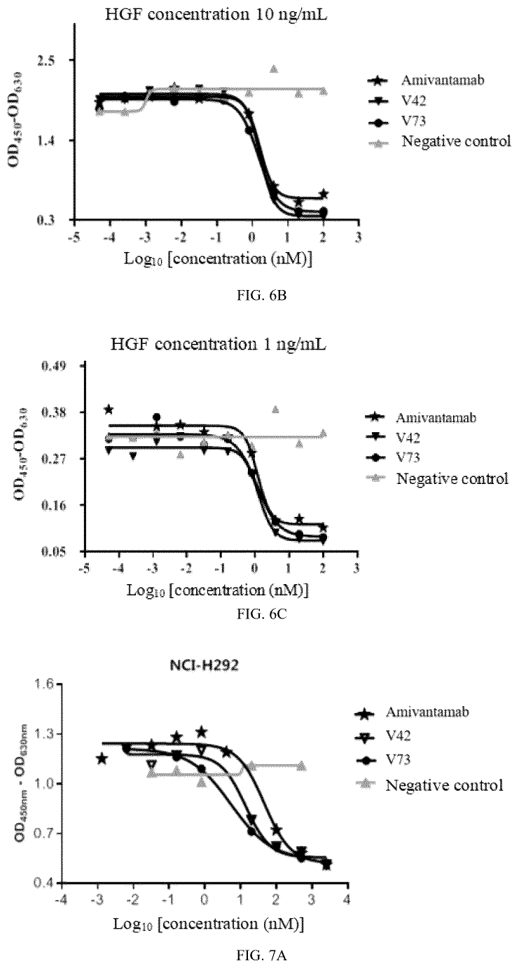

FIGS. 6A to 6C show the activity of anti-EGFR/anti-c-Met multispecific antibodies in competing with HGF ligand for binding to c-Met. The results demonstrated that the activity of V42 and V73 in competing with HGF ligand for binding to c-Met was substantially comparable to that of amivantamab.

### Example 13. Inhibitory Activity of Anti-EGFR/Anti-c-Met Multispecific Antibodies Against Tumor Cell Proliferation

The inhibitory activity of the anti-EGFR/anti-c-Met multispecific antibodies (including amivantamab, V42, and V73) against tumor cell proliferation was analyzed on a microplate reader, with an hIgG1 (Biointron, Cat. No. B117901) as the negative control.

NCI-H292 cells (medium EGFR expression and low to medium c-Met expression, from BeNa Culture Collection) and KP4 (medium EGFR expression and low c-Met expression, from Cobioer Biosciences) in the logarithmic growth phase were taken, adjusted to a viable cell density of 1.5×10⁴ to 2× 10⁴ cells/mL using an RPMI medium (Hyclon, Cat. No. SH30809.01) containing 2% of fetal bovine serum, and added to a 96-well plate (Costar, Cat. No. 3599) at 100 μL/well. The anti-EGFR/anti-c-Met multispecific antibodies were prepared in an RPMI medium, with a highest concentration of 2500 nM followed by 5-fold serial dilutions to yield a total of 10 concentration levels for NCI-H292 cells, and a highest concentration of 555 nM followed by 3-fold serial dilutions to yield a total of 8 concentration levels for KP4 cells. The antibodies at different concentrations were added to the 96-well cell culture plate at 100 μL/well. The plate was incubated in a thermostat incubator at 37° C. with 5% CO₂. For NCI-H292 cells, 50 μL of HGF was added to the culture medium at a final concentration of 1 ng/mL, and the plate was incubated for 144 h. For KP4, the plate was incubated for 120 h. A CCK8 working solution was added to the cell culture plate at 20 μL/well, and the plate was incubated in a thermostat incubator at 37 °C with 5% CO₂ for 2 h. The absorbance values at 450 nm were read on a microplate reader with 630 nm as the reference wavelength.

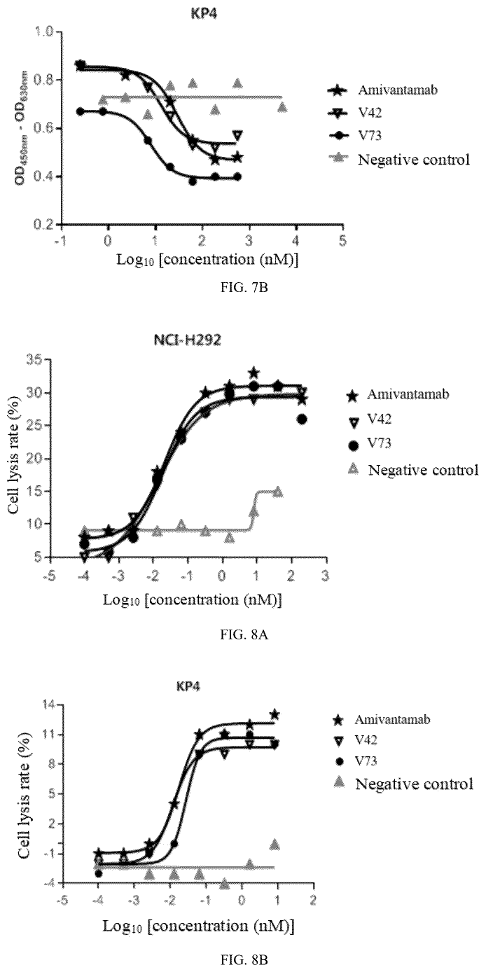

FIGS. 7A and 7B, and Table 8 show the inhibitory activity of anti-EGFR/anti-c-Met multispecific antibodies against tumor cell proliferation. The results demonstrated that in NCI-H292 cells and KP4 cells, the inhibitory activity of V73 and V42 against tumor cell proliferation was superior to that of amivantamab.

TABLE 8
IC₅₀ values for inhibiting the proliferation of
NCI-H292 and KP4 cells by the antibodies

| | NCI-H292 | KP4 |
|---|---|---|
| | IC₅₀ (nM) | IC₅₀ (nM) |
| V42 | 13.72 | 13.09 |
| V73 | 5.356 | 7.985 |
| Amivantamab | 45.78 | 28.96 |

### Example 14. ADCC Effects of Anti-EGFR/Anti-c-Met Multispecific Antibodies on Tumor Cells

The antibody-dependent cell-mediated cytotoxicity (ADCC) effects of the anti-EGFR/anti-c-Met multispecific antibodies (including amivantamab, V42, and V73) on tumor cells were explored using human PBMCs (peripheral blood mononuclear cells), with an hIgG1 (Biointron, Cat. No. B117901) as the negative control. NCI-H292 and KP4 cells in the logarithmic growth phase were taken, and adjusted to a viable cell density of 3×10⁵ cells/mL with an RPMI medium (Hyclon, Cat. No. SH30809.01) containing 2% of fetal bovine serum to give target cells. The anti-EGFR/anti-c-Met multispecific antibodies were prepared in an RPMI medium, with a highest concentration of 8-200 nM followed by 5-fold serial dilutions to yield a total of 8-10 concentration levels. PBMC cells were thawed, counted, and adjusted to a PBMC viable cell density of 1.5×10⁶ cells/mL with an RPMI medium to give effector cells. A treatment group (50 μL of target cells+100 μL of effector cells+50 μL of antibody), a target cell group (50 μL of target cells+150 μL of culture medium), an effector cell group (100 μL of effector cells+100 μL of culture medium), a target+ effector cell group (50 μL of target cells+100 μL of effector cells+50 μL of culture medium), a blank control group (200 μL of culture medium), a lysis buffer control group (200 μL of culture medium+20 μL of lysis buffer), and a target cell maximum release group (50 μL of target cells+150 μL of culture medium+20 μL of lysis buffer) were set. The groups were added to a 96-well plate (Costar, Cat. No. 3599) in an effector-to-target cell ratio of 10:1. The cells were incubated in a thermostat incubator at 37 °C with 5% CO₂ for 24 h. The cell lysis rates were measured using a CytoTox96® nonradioactive cytotoxicity assay kit (Promega, Cat. No. G1780). Finally, the absorbance at 490 nm was measured on a microplate reader.

$$Cell lysis rate ( \% ) = \frac{{OD}_{Treatment group} - {OD}_{Target + effector cell group}}{\begin{matrix}{OD}_{Target cell maximum release group} - \\ {OD}_{Target cell group}\end{matrix}} \times 100 \%$$

FIGS. 8A and 8B, and Table 9 show the ADCC effects of anti-EGFR/anti-c-Met multispecific antibodies on tumor cells.

TABLE 9
Lysis EC₅₀ values and maximum lysis rates of
the antibodies to NCI-H292 and KP4 cells

| | NCI-H292 | | KP4 | |
|---|---|---|---|---|
| | EC₅₀ | Maximum | EC₅₀ | Maximum |
| | (nM) | lysis rate | (nM) | lysis rate |
| V42 | 0.01346 | 30% | 0.01166 | 11% |
| V73 | 0.01813 | 31% | 0.02699 | 11% |
| Amivantamab | 0.02105 | 33% | 0.01708 | 13% |

### Example 15. Internalization Activity of Anti-EGFR/Anti-c-Met Multispecific Antibodies in Tumor Cells

The internalization activity of the anti-EGFR/anti-c-Met multispecific antibodies (including amivantamab, V42, and V73) in A431 cells expressing EGFR and c-Met, NCI-H1975 cells, and NCI-H441 cells (medium EGFR expression and medium c-Met expression, from BeNa Culture Collection) was analyzed by flow cytometry, with an hIgG1 (Biointron, Cat. No. B117901) as the negative control.

A431 cells, NCI-H1975 cells, and NCI-H441 cells in the logarithmic growth phase were separately adjusted to a viable cell density of 2× 10⁶ cells/mL using a complete medium, and seeded in 96-well V-bottom cell culture plates (Costar, Cat. No. 3894) at 20 μL/well. A complex of the anti-EGFR/anti-c-Met multispecific antibody and the Antibody Internalization Human Reagent (Sartorius, Cat. No. 90564) was prepared according to the instructions of the Antibody Internalization Human Reagent, such that the final concentration of the anti-EGFR/anti-c-Met multispecific antibody was 100-167 nM. The mixture was incubated at 37 °C for 15 min, and 5-fold serially diluted to yield a total of 10 concentration levels. The complex solution was added to the cell culture plate at 20 μL/well. The mixture was mixed thoroughly and then incubated in a thermostat incubator at 37 °C with 5% CO₂ for 2 h. The changes in fluorescence values were detected in the RLI channel of the iQue3 flow cytometer, and the data were analyzed using software.

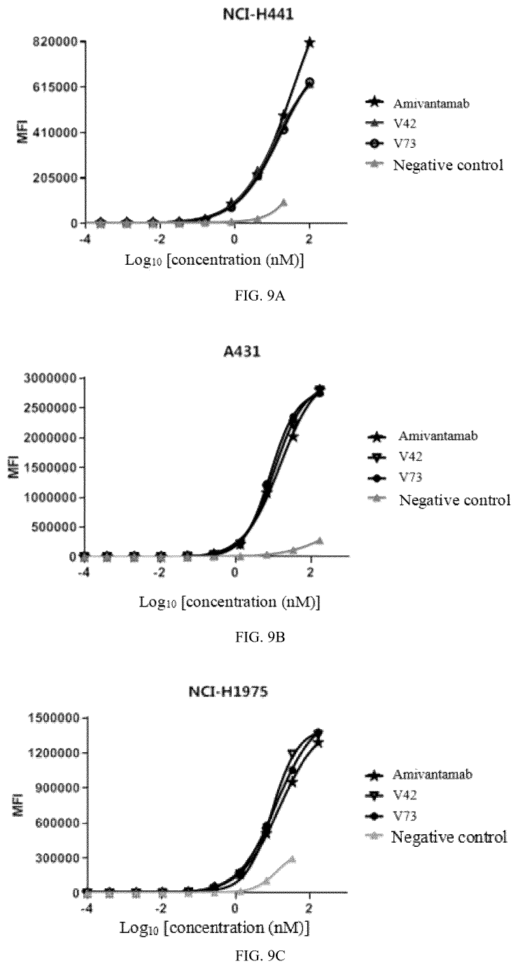

FIGS. 9A to 9C and Table 10 show the internalization activity of the anti-EGFR/anti-c-Met multispecific antibodies in A431 cells, NCI-H1975 cells, and NCI-H441 cells.

TABLE 10
Internalization activity of anti-EGFR/anti-c-Met multispecific
antibodies in A431 cells, NCI-H1975 cells, and NCI-H441 cells

| | NCI-H441 | | A431 | | NCI-H1975 | |
|---|---|---|---|---|---|---|
| | Top (MFI) | EC₅₀ (nM) | Top (MFI) | EC₅₀ (nM) | Top (MFI) | Top (MFI) |
| V42 | 11.81 | 629221 | 10.54 | 2797694 | 9.662 | 1356725 |
| V73 | 16.72 | 638113.5 | 8.333 | 2745828 | 12.43 | 1376844 |
| Amivantamab | 35.63 | 815804 | 15.06 | 2799466.5 | 15.16 | 1290047 |
| Negative control | / | 97506 | / | 117841.5 | / | 110471 |

### Example 16. Pharmacodynamic Evaluation of Anti-EGFR/Anti-c-Met Multispecific Antibodies in U-87 MG Human Neuroglioma Nude Mouse Subcutaneous Xenograft Tumor Model

SPF-grade female BALB/c-nu nude mice (purchased from Beijing HFK Bioscience Co., Ltd.) were subcutaneously grafted with U-87 MG cells (ATCC HTB-14TM). After the tumor grew to 100-150 mm³, the mice were divided into a modeling group (normal saline), V42 groups, and V73 groups, with 8 mice in each group. The day of the first dose was day 0 (DO). The drug (0.25 mg/kg or 0.75 mg/kg) was administered intravenously (i.v.) twice a week for a total of 6 doses at an injection volume of 0.1 mL/10 g mouse body weight. The tumors were measured twice weekly with a vernier caliper. The pharmacodynamic evaluation was conducted as per the tumor growth inhibition rate (TGI).

The measurement parameters and the calculation formulas are as follows:
Tumor volume (mm³)=½×(a×b²) (where a represents the long diameter and b represents the short diameter of the tumor).
Relative tumor proliferation rate T/C (%)=(T−T₀)/(C−C₀)×100%,
TGI (%)=100%−T/C, where T and C are the tumor volumes of the treatment and modeling groups, respectively, at the end of the study; T₀ and C₀ are the tumor volumes of the treatment and modeling groups, respectively, at the start of the study.

If the tumor volume was smaller than the tumor volume at the start of the study, i.e., T<T₀ or C<C₀, it was defined as partial regression (PR) of the tumor. When the tumor regressed, TGI (%)=100−(T−T₀)/T₀×100%. The results of the measurement parameters are shown in Table 11. In the U-87 MG human neuroglioma nude mouse subcutaneous xenograft tumor model, V42 and V73 both exhibited significant anti-tumor effects.

TABLE 11
Effect of anti-EGFR/anti-c-Met multispecific antibodies on U-87 MG
human neuroglioma subcutaneous xenograft tumors in nude mice

| | Tumor volume | Tumor volume | | | |
|---|---|---|---|---|---|
| | on D0 | on D20 | | | |
| | (mm³) | (mm³) | D20 | D20 | |
| Group | (Mean ± SEM) | (Mean + SEM) | T/C | TGI | P value |
| Modeling group | 115.4 ± 1.7 | 2014 ± 234.4 | — | — | — |
| V42 group (0.25 mg/kg) | 115.7 ± 1.5 | 1352.6 ± 228.4 | 65% | 35% | 0.062 |
| V42 group (0.75 mg/kg) | 115.8 ± 1.4 | 210.9 ± 38.4 | 5% | 95% | <0.001 |
| V73 group (0.25 mg/kg) | 116.1 ± 1.5 | 1774.3 ± 134.4 | 87% | 13% | 0.388 |
| V73 group (0.75 mg/kg) | 116.6 ± 1.2 | 207.7 ± 69 | 5% | 95% | <0.001 |

P value is determined by comparison with the modeling group.

### Example 17: Preparation of Linker-Payload MC-GGFG-Deuterated DXd (MC-GGFG-DDDXd)

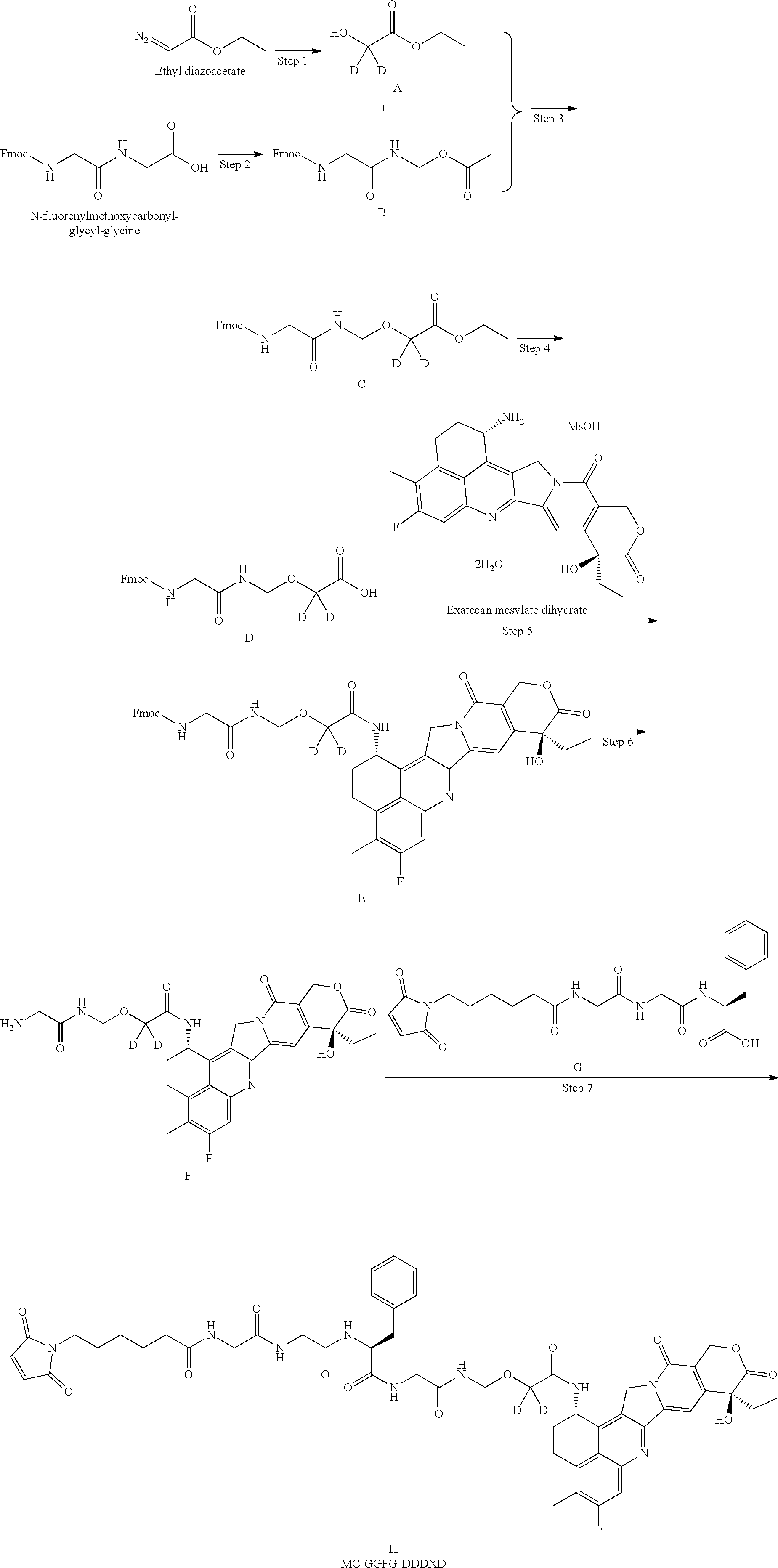

### Step 1: Synthesis of Intermediate A

80 g of ethyl diazoacetate was added to a 3 L single-neck flask under a nitrogen atmosphere, and 800 mL of dichloromethane and 800 mL of 1% deuterated acetic acid solution (8 g of deuterated acetic acid dissolved in 800 mL of deuterium oxide) were added. The single-neck flask was stirred at room temperature for 75 h under light-shielding conditions. Liquid separation was performed. The organic phase was collected, and the aqueous phase was extracted twice with dichloromethane (200 mL×2). The organic phases were combined. The combined organic phase was washed with 200 mL of deuterium oxide. The resulting organic phase was dried over anhydrous sodium sulfate and filtered to remove sodium sulfate. The filtrate was concentrated to dryness under reduced pressure at 20 °C to obtain 49.25 g of intermediate A. CAS of intermediate A: 1356471-71-0.

### Step 2: Synthesis of Intermediate B

50 g of N-fluorenylmethoxycarbonyl-glycyl-glycine was weighed and added to a 2 L round-bottom flask, and 750 mL of tetrahydrofuran and 150 mL of glacial acetic acid were added. The mixture was stirred at 40 °C for 20 min, and 100 g of lead tetraacetate was added. The mixture was heated to 80 °C and then reacted for 3 h. The reaction solution was cooled to room temperature and filtered under vacuum. The filter cake was washed with 250 mL of ethyl acetate. The filtrate was concentrated to dryness and dissolved with 330 mL of dichloromethane and 670 mL of ethyl acetate to obtain the organic phase. The organic phase was washed 3 times with 30% aqueous potassium bicarbonate (500 mL×3), then dried over anhydrous sodium sulfate, and filtered. The filtrate was concentrated to dryness under reduced pressure, and dissolved with 100 mL of dichloromethane. Then, 100 mL of n-hexane was added. The mixture was stirred at room temperature until a solid was precipitated, and then 300 mL of a mixed solution of n-hexane and dichloromethane (n-hexane: dichloromethane=1:1) was added. The mixture was stirred overnight. The reaction solution was filtered. The filter cake was dried in a vacuum oven at 40 °C for 4 h to obtain 37.3 g of intermediate B. LC-MS (ESI) m/z: 391.09 [M+Na]⁺; CAS of intermediate B: 1599440 June 8.

### Step 3: Synthesis of Intermediate C

78 g of intermediate B was weighed and added to a 3000 mL single-neck flask, and 800 mL of dichloromethane and 45 g of intermediate A were added thereto. The 3000 mL single-neck flask was placed in an ice-water bath to be cooled to 0 °C. 16 g of lithium tert-butoxide was dissolved in 400 mL of dichloromethane to prepare a lithium tert-butoxide solution, which was then added to a 3000 mL single-neck flask and reacted at 0 °C for 3 h. The reaction solution was warmed to room temperature, and 800 mL of water was added. The mixture was stirred. Liquid separation was performed. The organic phase was collected, and the aqueous phase was extracted with 400 mL of dichloromethane. The organic phases were combined. The combined organic phase was washed once with 800 mL of saturated brine, dried over anhydrous sodium sulfate, and filtered under vacuum. The filtrate was concentrated under reduced pressure and subjected to silica gel column chromatography (petroleum ether:ethyl acetate=3:1) to give 61 g of intermediate C. LC-MS (ESI) m/z: 437.34 [M+Na]⁺; CAS of intermediate C: 2760715-83-9.

### Step 4: Synthesis of Compound D

31.2 g of intermediate C was added to 270 mL of deuterated methanol and 70 mL of deuterium oxide. The mixture was stirred in an ice bath, and then 5.5 g of NaOH was added. The mixture was warmed to room temperature and stirred overnight. 300 mL of ethyl acetate and 300 mL of water were added to the reaction solution for extraction. 20 mL of glacial acetic acid was added to the aqueous layer to adjust the pH to 2-3, and a solid was precipitated. The mixture was filtered under vacuum to obtain 20.3 g of compound D. ¹H-NMR (500 MHz, DMSO-d6) δ 8.70 (t, J=6.6 Hz, 1H), 7.89 (d, J=7.5 Hz, 2H), 7.72 (d, J=7.5 Hz, 2H), 7.57 (t, J=6.0 Hz, 1H), 7.42 (t, J=7.5 Hz, 2H), 7.34 (t, J=7.5 Hz, 2H), 4.61 (d, J=6.6 Hz, 2H), 4.30 (d, J=7.1 Hz, 2H), 4.23 (1H, m), 3.64 (d, J=6.0 Hz, 2H); LC-MS (ESI) m/z: 409.08 [M+Na]⁺.

### Step 5: Synthesis of Compound E

2.0 g of exatecan mesylate dihydrate and 1.63 g of compound D were weighed and added to a 100 mL round-bottom flask. 40 mL of N,N-dimethylformamide was added. The mixture was stirred. The reaction solution was cooled to 0 °C. 2.0 g of O-(7-azabenzotriazol-1-yl)-N,N,N′,N′-tetramethyluronium hexafluorophosphate and 1.82 g of N,N-diisopropylethylamine were added in sequence. The mixture was reacted at 0 °C for 3 h. The reaction solution was poured into 120 mL of ice water. The mixture was stirred for 1 h. The reaction solution was filtered. The filter cake was dissolved with dichloromethane. The reaction solution was subjected to silica gel column chromatography (100 g of 100-200 mesh silica gel, dichloromethane:methanol=30:1, 2 L) to obtain 2.6 g of compound E. ¹H-NMR (500 MHz, DMSO-d₆), δ 8.78 (t, J=6.6 Hz, 1H), 8.47 (d, J=9.0 Hz, 1H), 7.86 (d, J=7.5 Hz, 2H), 7.73 (d, J=11.0 Hz, 1H), 7.68 (d, J=7.5 Hz, 2H), 7.53 (t, J=6.0 Hz, 1H), 7.39 (t, J=7.5 Hz, 2H), 7.30 (m, 2H), 7.29 (s, 1H), 6.50 (brs, 1H), 5.56 (m, 1H), 5.39 (m, 2H), 5.14 (m, 2H), 4.64 (m, 2H), 4.25 (d, J=6.8 Hz, 2H), 4.19 (m, 1H), 3.62 (d, J=6.0 Hz, 2H), 3.15 (m, 2H), 2.35 (s, 3H), 2.17 (m, 2H), 1.83 (m, 2H), 0.85 (t, J=7.3 Hz, 3H); LC-MS (ESI) m/z: 804.84 [M+H]⁺.

### Step 6: Preparation of Compound F

0.38 g of 1,8-diazabicyclo[5.4.0]undec-7-ene was weighed and added to a 100 mL round-bottom flask, and then 20 mL of tetrahydrofuran was added to the round-bottom flask. The mixture was stirred. The reaction solution was cooled to 0 °C. 2.0 g of compound E was weighed and prepared into a solution with 20 mL of tetrahydrofuran. The prepared compound E solution was slowly added to the 100 mL round-bottom flask. The mixture naturally heated to room temperature and reacted for 3 h. The reaction solution was filtered under a nitrogen atmosphere to give 1.45 g of compound F. ¹H-NMR (500 MHz, DMSO-d₆) δ 8.76 (m, 1H), 7.72 (m, 1H), 7.28 (s, 1H), 5.48 (m, 3H), 5.16 (m, 2H), 4.61 (m, 2H), 3.40 (m, 2H), 3.20 (m, 2H), 2.35 (s, 3H), 2.17 (m, 2H), 1.83 (m, 2H), 0.86 (t, J=7.1 Hz, 3H); LC-MS (ESI) m/z: 582.39 [M+H]⁺.

### Step 7: Preparation of Compound H

1.00 g of compound F and 0.97 g of compound G were weighed and added to a 100 mL round-bottom flask, and 10 mL of N,N-dimethylformamide was added. The mixture was cooled to −20° C., and 0.34 g of 1-hydroxybenzotriazole and 0.49 g of 1-(3-dimethylaminopropyl)-3-ethylcarbodiimide hydrochloride were added. The mixture was reacted at −20 °C for 3 h. 20 mL of dichloromethane and 20 ml of water were added to the reaction solution. The mixture was stirred for 0.5 h and left to stand for liquid separation. The organic phase was collected. The organic phase was dried over anhydrous sodium sulfate and filtered. The filtrate was concentrated to dryness under reduced pressure. The residue was subjected to silica gel column chromatography (dichloromethane:methanol=15:1) to obtain 400 mg of compound H. MS m/z: 1037.08 [M+H]⁺. ¹H-NMR (500 MHz, DMSO-d₆) δ 8.62 (t, J=6.5 Hz, 1H), 8.49 (d, J=8.5 Hz, 1H), 8.29 (t, J=5.5 Hz, 1H), 8.12 (d, J=8.0 Hz, 1H), 8.06 (t, J=5.5 Hz, 1H), 8.00 (t, J=5.5 Hz, 1H), 7.74 (d, J=10.5 Hz, 1H), 7.30 (s, 1H), 7.27-7.12 (m, 5H), 6.98 (s, 2H), 6.51 (brs, 1H), 5.61-5.58 (m, 1H), 5.45-5.37 (m, 2H), 5.22-5.13 (m, 2H), 4.64 (d, J=6.5 Hz, 2H), 4.49-4.45 (m, 1H), 3.76-3.57 (m, 6H), 3.37-3.32 (m, 2H), 3.24-3.09 (m, 2H), 3.02 (dd, J=4.5 Hz, 14.0 Hz, 1H), 2.77 (dd, J=9.5 Hz, 13.5 Hz, 1H), 2.36 (s, 3H), 2.23-2.14 (m, 2H), 2.09 (t, J=7.5 Hz, 2H), 1.91-1.79 (m, 2H), 1.49-1.42 (m, 4H), 1.20-1.14 (m, 2H), 0.87 (t, J=7.5 Hz, 3H); HR-MS m/z: 1036.4194 [M+H]⁺.

### Example 18: Preparation of ADCs Targeting EGFR and c-Met

### Example 18-1: Preparation of ADC V42-DDDXd-6 (1) Targeting EGFR and c-Met Reagents

The antibody was the anti-EGFR/anti-c-Met multispecific antibody V42, and the linker-payload was MC-GGFG-DDDXd synthesized in Example 17.

### Experimental Procedures:

1. Reduction of antibody: The concentration of the antibody was adjusted to about 8-10 g/L with 20 mM histidine buffer (containing 1.43 mg/mL L-histidine, 2.27 mg/mL L-histidine hydrochloride monohydrate, 5% sucrose, pH 6.0), and then the pH was adjusted to about 6.0 with 0.3 M aqueous disodium hydrogen phosphate solution. The temperature of the above solution was adjusted to 30° C., a 10 mM aqueous TCEP.HCl (tris(2-carboxyethyl) phosphine hydrochloride) solution was added to the solution to enable the molar ratio of TCEP. HCl to the antibody to be 6.0:1, and the mixture was reacted in the dark at 30 °C with stirring at 50-60 rpm for 2 h to obtain a reaction solution 1.

2. Conjugation of antibody to linker-payload: DMSO was added to the reaction solution 1 to a final concentration of 5% (v/v), and then linker-payload dissolved in DMSO at a concentration of 10 g/L was added to enable the molar ratio of the linker-payload to the antibody to be 9.25:1. The mixture was reacted in the dark at 22 °C with stirring at 50-60 rpm for 2 h to obtain a reaction solution 2.

3. Conjugation termination and purification: After the conjugation was completed, the reaction solution 2 was exchanged into a 20 mM histidine buffer (20 g/kg polysorbate 80 (II), 0.753 g/kg histidine, 3.1762 g/kg histidine hydrochloride, pH 5.8) using a 30 kDa ultrafiltration membrane to obtain an antibody-drug conjugate V42-DDDXd-6 (1).

Referring to the method of Example 19, the DAR value of V42-DDDXd-6 (1) was determined to be 6.0. The structure of V42-DDDXd-6 (1) is as follows:

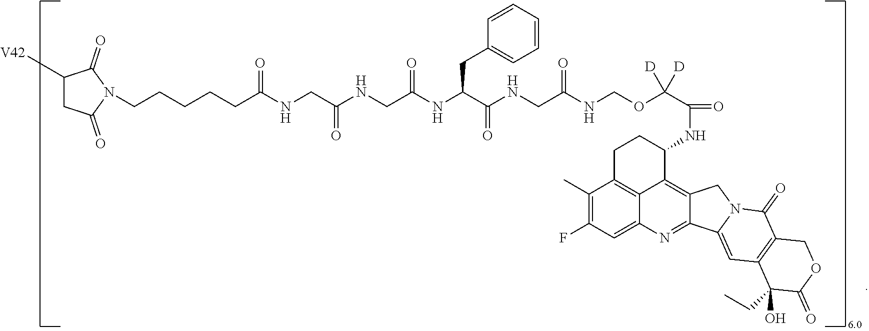

### Example 18-2: Preparation of ADC V42-DDDXd-6 (2) Targeting EGFR and c-Met Reagents

The antibody was the anti-EGFR/anti-c-Met multispecific antibody V42, and the linker-payload was MC-GGFG-DDDXd synthesized in Example 17.

### Experimental Procedures:

1. Reduction of antibody: The concentration of the antibody was adjusted to about 18-20 g/L with 20 mM histidine buffer (containing 1.43 mg/mL L-histidine, 2.27 mg/mL L-histidine hydrochloride monohydrate, 5% sucrose, pH 6.0), and then the pH was adjusted to about 6.5 with 0.3 M aqueous disodium hydrogen phosphate solution. The temperature of the above solution was adjusted to 30° C., a 10 mM aqueous TCEP.HCl (tris(2-carboxyethyl)phosphine hydrochloride) solution was added to the solution to enable the molar ratio of TCEP. HCl to the antibody to be 6.0:1, and the mixture was reacted in the dark at 30 °C with stirring at 50-60 rpm for 2 h to obtain a reaction solution 1.

2. Conjugation of antibody to linker-payload: DMSO was added to the reaction solution 1 to a final concentration of 5% (v/v), and then linker-payload dissolved in DMSO at a concentration of 10 g/L was added to enable the molar ratio of the linker-payload to the antibody to be 9.25:1. The mixture was reacted in the dark at 22 °C with stirring at 50-60 rpm for 2 h to obtain a reaction solution 2.

3. Conjugation termination and purification: After the conjugation was completed, the reaction solution 2 was exchanged into a 20 mM histidine buffer (pH 5.8) using a 30 kDa ultrafiltration membrane to obtain an antibody-drug conjugate V42-DDDXd-6 (2).

Referring to the method of Example 19, the DAR value of V42-DDDXd-6 (2) was determined to be 5.9. The structure of V42-DDDXd-6 (2) is as follows:

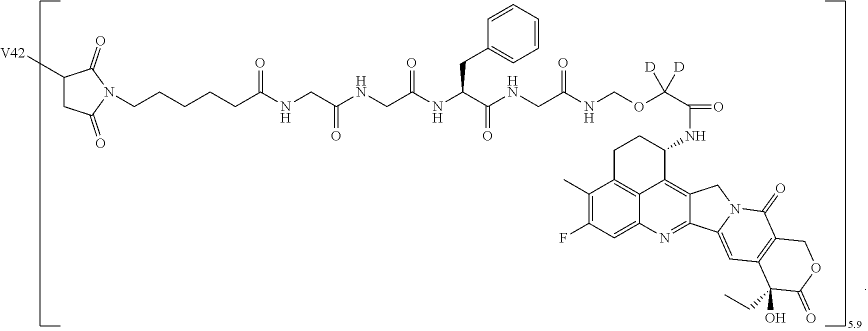

### Example 18-3: Preparation of ADC Reference (Benchmark) AZD9592 Targeting EGFR and c-Met Reagents

Antibody: The antibody was RAA22/B09-57; the linker-payload was Mal-PEG8-amide-Val-Ala-(4-NH₂)-Exatecan (AZ0133, manufacturer: MCE, Cat. No. HY-145399).

### Experimental Procedures:

1. Reduction of antibody: The antibody was exchanged into a histidine buffer (1.43 mg/mL L-histidine and 2.27 mg/mL L-histidine hydrochloride monohydrate) at pH 6.0, and the antibody concentration was adjusted to 10 mg/mL with the histidine buffer at pH 6.0. The antibody solution was added to a light-protected glass vial, and a 10 mM TCEP HCl (tris(2-carboxyethyl)phosphine hydrochloride) solution was added to enable the molar ratio of the antibody to TCEP to be 1:10. The mixture was incubated at 37 °C for 1 h in the dark with gentle stirring, so as to reduce the inter-chain disulfide bonds of the antibody to obtain a reaction solution 1.

2. Conjugation of antibody to linker-payload: A linker-payload solution at a concentration of 10 mg/mL, which was dissolved with DMSO, was added to the reaction solution 1 to enable the molar ratio of the antibody to the linker-payload to be 1:12. The mixture was incubated at 22 °C for 1 h in the dark with gentle stirring, so as to conjugate the antibody with the linker-payload to obtain a reaction solution 2.

3. Conjugation termination and purification: The reaction solution 2 was ultrafiltered with a histidine buffer at pH 6.0 to obtain an antibody-drug conjugate AZD9592.

Referring to the method of Example 19, the DAR value of AZD9592 was determined to be 6.0.

The structure of AZD9592 is as follows:

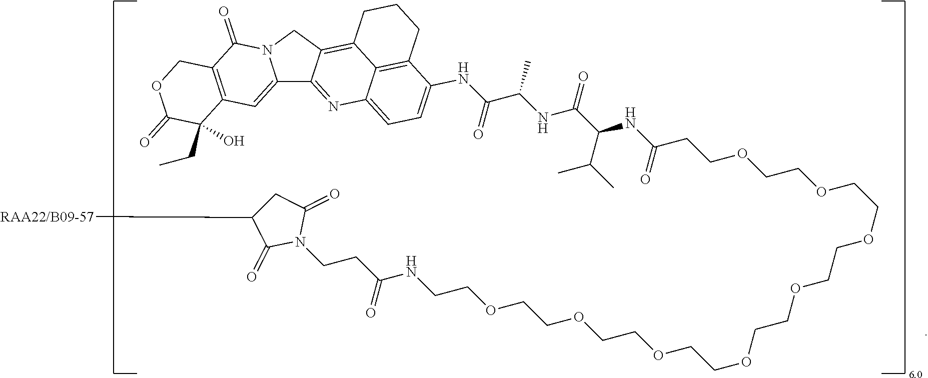

### Example 19. Determination of DAR Value of Antibody-Drug Conjugates

The DAR value of the ADCs was determined by a hydrophobic interaction chromatography (HIC) method. The hydrophobic property of the protein molecules was improved using a neutral high-salt mobile phase, thereby allowing binding to the hydrophobic bonds in the chromatographic column, and then the substances were eluted by gradually decreasing the salt concentration and gradually increasing the proportion of isopropanol, with components of lower hydrophobicity eluting first and those of higher hydrophobicity eluting later.

Components of ADCs targeting EGFR and c-Met were separated using butyl-bonded nonporous polystyrene/divinylbenzene (PS/DVB) packing. The specification of the chromatographic column was Sepax HIC-Butyl, 4.6×100 mm, 5 μm, and the column temperature was 25 °C. The mobile phase A was 25 mmol/L phosphate buffered saline-2 mol/L ammonium sulfate at pH 7.0 (3.55 g of anhydrous disodium hydrogen phosphate and 264.28 g of ammonium sulfate were weighed out, added to about 800 mL of ultrapure water, and dissolved completely by stirring, then the pH was adjusted to 7.0±0.1 with phosphoric acid, the volume was brought to 1 L, and the mixture was mixed thoroughly and filtered through a 0.22 μm filter membrane). The mobile phase B was 25 mmol/L phosphate buffered saline at pH 7.0 (3.55 g of anhydrous disodium hydrogen phosphate was weighed out, added to about 800 mL of ultrapure water, and dissolved completely by stirring, then the pH was adjusted to 7.0±0.1 with phosphoric acid, the volume was brought to 1 L, and the mixture was mixed thoroughly and filtered through a 0.22 μM filter membrane). Mobile phase C was 100% isopropanol. The ADC sample was diluted 1:1 with the initial-ratio mobile phase to obtain a test solution. Based on the sample concentration, the injection volume was adjusted such that 50 μg of protein was injected. Detection was performed at the wavelength of 280 nm. The flow rate was 0.5 mL/min, and gradient elution was performed for 30 min. The elution procedure is shown in Table 12.

Data were processed, and the results were quantitatively analyzed using the area normalization method. The percentages of ADC peak areas for containing 0, 1, 2, 3, 4, 5, 6, 7, and 8 cytotoxic drugs were calculated separately, and DAR values were calculated. The calculation formula is as follows: DAR value=(percentage of ADC peak area for containing 0 cytotoxic drugs×0+percentage of ADC peak area for containing 1 cytotoxic drug×1+percentage of ADC peak area for containing 2 cytotoxic drugs×2+percentage of ADC peak area for containing 3 cytotoxic drugs×3+percentage of ADC peak area for containing 4 cytotoxic drugs×4+percentage of ADC peak area for containing 5 cytotoxic drugs×5+percentage of ADC peak area for containing 6 cytotoxic drugs×6+percentage of ADC peak area for containing 7 cytotoxic drugs×7+percentage of ADC peak area for containing 8 cytotoxic drugs×8)/100%.

TABLE 12
Gradient elution parameters

| | Mobile | Mobile | Mobile |
|---|---|---|---|
| Time | phase A | phase B | phase C |
| 0 min | 75% | 25% | / |
| 15.0 min | 10% | 65% | 25% |
| 15.1 min | / | 100% | / |
| 20 min | / | 100% | / |
| 20.1 min | 75% | 25% | / |
| 30 min | 75% | 25% | / |

### Example 20. Aggregate verification for antibody-drug conjugates

Components of the ADC samples targeting EGFR and c-Met were separated by gel column chromatography. Elution was performed using a neutral buffer containing 10% isopropanol as the mobile phase. The components were eluted sequentially in descending order based on their molecular weights. The gel chromatographic column used was an ACQUITY UPLC Protein BEH SEC Column 200 Å, 1.7 μm, 4.6×300 mm, and the column temperature was 25 °C. The mobile phase was 50 mmol/L phosphate buffered saline-200 mmol/L sodium chloride-10% isopropanol at pH 7.0 (12.53 g of disodium hydrogen phosphate dodecahydrate, 2.33 g of sodium dihydrogen phosphate dihydrate, and 11.69 g of sodium chloride were weighed out, added to about 800 mL of ultrapure water, and dissolved completely by stirring; ultrapure water was added to bring the volume to 1000 mL for use; after the mixture was mixed thoroughly, 900 mL of the solution was taken and 100 mL of isopropanol was added thereto; and the mixture was mixed thoroughly and then filtered through a 0.22 μm filter membrane). 20 μg of the ADC sample was precisely taken and injected into a liquid chromatograph, and detection was performed at the wavelength of 280 nm. The flow rate was 0.3 mL/min, and isocratic elution was performed for 15 min. Data were processed, and the results were quantitatively analyzed using the area normalization method. The peak area percentages for the aggregate, immunoglobulin monomer, and low molecular weight impurity were calculated. The aggregate peak appeared before the main peak, the main peak represented the immunoglobulin monomer, and the low molecular weight impurity peaks appeared after the main peak. The assay results are shown in Table 13.

TABLE 13
Content of monomers, aggregates, and low molecular weight
impurities in ADCs targeting EGFR and c-Met

| | SEC | | |
|---|---|---|---|
| | | | Low molecular |
| | Aggregates | Monomers | weight impurities |
| Sample name | (%) | (%) | (%) |
| V42-DDDXd-6(1) | 0.45 | 96.63 | 2.92 |
| V42-DDDXd-6(2) | 1.16 | 96.11 | 2.73 |
| AZD9592 | 1.06 | 93.87 | 5.07 |

### Example 21: Affinity of ADCs Targeting EGFR and c-Met for Human EGFR and Human c-Met

The affinity of the ADCs for human EGFR-His protein or human c-Met-His protein was assayed using a biomolecular interaction analysis system (Cytiva, Biacore 8K). An anti-hIgG (Fc) antibody (Cytiva, Cat. No. BR-1008-39) was amino-coupled to a CM5 sensor chip. The antibody was diluted to 4 μg/mL with a running buffer (137 mM NaCl, 2.7 mM KCl, 10 mM Na₂HPO₄·12H₂O, 1.8 mM KH₂PO₄, 0.05% surfactant P-20 (w/v), pH 7.4), and the capture was conducted by passing the dilutions through experimental channels at a flow rate of 30 μL/min. Human EGFR-His or human c-Met-His was diluted with the running buffer to 200 nM, 100 nM, 50 nM, 25 nM, 12.5 nM, 6.25 nM, and 3.125 nM, and the association was conducted for 200 s by passing the dilutions at a flow rate of 50 μL/min. Then dissociation was conducted for 600 s by stopping sample injection. Data signals were collected in real time, and the data were analyzed using software. The Langmuir 1:1 model was used for fitting, and the association rate constant kₐ (1/Ms), the dissociation rate constant k_{d} (1/s), and the equilibrium dissociation constant K_{D} (M) were calculated. The assay results are shown in Table 14.

TABLE 14
Affinity of antibody-drug conjugates

| ADC | Protein | kₐ (1/Ms) | k_{d} (1/s) | K_{D} (M) |
|---|---|---|---|---|
| V42-DDDXd-6(1) | Human | 5.36E+04 | 2.18E−04 | 4.07E−09 |
| AZD9592 | EGFR-His | 2.16E+04 | 1.06E−03 | 4.89E−08 |
| V42-DDDXd-6(1) | Human | 4.28E+04 | 2.91E−05 | 6.80E−10 |
| AZD9592 | c-MET-His | 1.10E+05 | 3.22E−04 | 2.94E−09 |

### Example 22: Cell Binding Activity of ADCs Targeting EGFR and c-Met

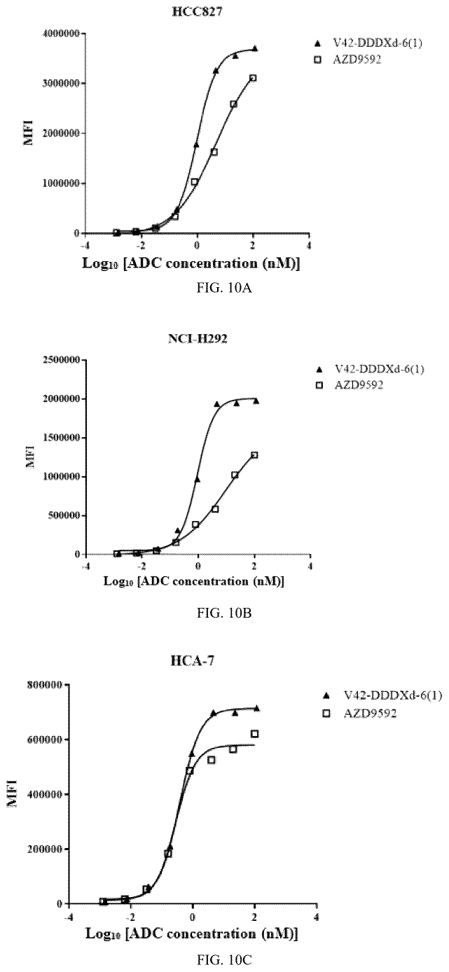

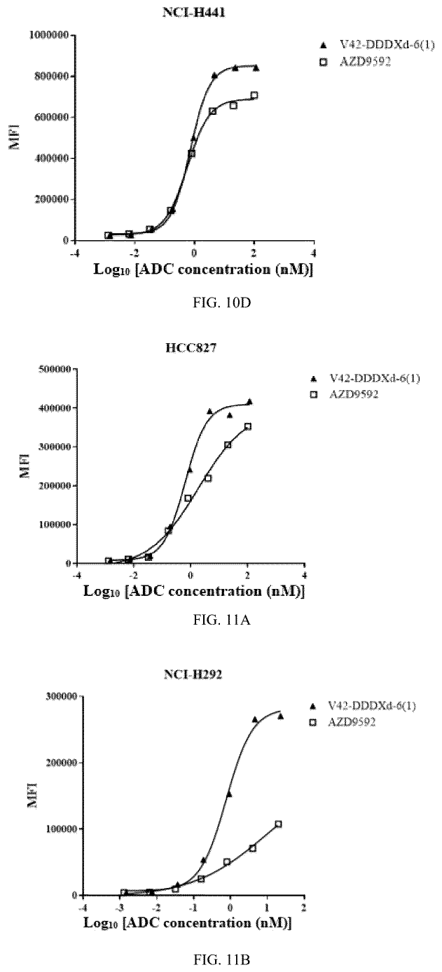

The cell binding activity of the ADCs targeting EGFR and c-Met was assayed by flow cytometry. Human non-small cell lung cancer cells HCC827 (high EGFR expression, low to medium c-Met expression, Yaji Biological, Cat. No. YS122C), NCI-H441, NCI-H292, and human colon adenocarcinoma cells HCA-7 (low to medium EGFR expression, low to medium c-Met expression, Nanjing Cobioer, Cat. No. CBP60035) were diluted to 2×10⁶ cells/mL and added to a 96-well plate at 50 μL/well. The ADCs targeting EGFR and c-Met serially diluted with FACS buffer (Miltenyi Biotec, Cat. No. 130-091-221) were added at 50 μL/well (V42-DDDXd-6 (1) at a final concentration of 115.4 nM as the initial concentration and AZD9592 at a final concentration of 100 nM as the initial concentration, which were 5-fold serially diluted to yield a total of 8 concentrations). The cells were incubated at 4 °C for 1 h and centrifuged at 1000 rpm for 5 min. The supernatant was discarded, and the cells were washed 3 times with pre-cooled FACS buffer. A 1:200 (v/v) diluted goat anti-human IgG Fcγ-PE secondary antibody (Jackson immunoresearch, Cat. No. 109-116-170) was added at 100 μL/well, and the cells were incubated at 4 °C for 20 min. The cells were washed 3 times with pre-cooled FACS buffer and resuspended in 50 μL of FACS buffer. Then, the fluorescence signals were analyzed and detected using a flow cytometer (Sartorius, iQUE3). The binding activity of ADCs targeting EGFR and c-Met to cell surface EGFR and c-Met was expressed by the mean fluorescence intensity (MFI) of staining. Data were analyzed using GraphPad Prism 5. The results are shown in FIGS. 10A to 10D, and the calculated EC₅₀ values are shown in Table 15 below. The results showed that V42-DDDXd-6 (1) could effectively bind to cells with different expression levels of EGFR and c-Met, and the binding activity was superior to that of AZD9592.

TABLE 15
Binding of ADCs targeting EGFR and c-Met to cells

| | HCC827 | | NCI-H292 | | HCA-7 | | NCI-H441 | |
|---|---|---|---|---|---|---|---|---|
| | Top | EC₅₀ | Top | EC₅₀ | Top | EC₅₀ | Top | |
| | (MFI) | (nM) | (MFI) | (nM) | (MFI) | (nM) | (MFI) | (nM) |
| V42-DDDXd-6(1) | 3689266 | 0.97 | 2005399 | 0.92 | 713809 | 0.37 | 852616 | 0.72 |
| AZD9592 | 3635774 | 4.62 | 1652415 | 9.27 | 580053 | 0.28 | 690433 | 0.57 |

### Example 23: Internalization Activity of ADCs Targeting EGFR and c-Met

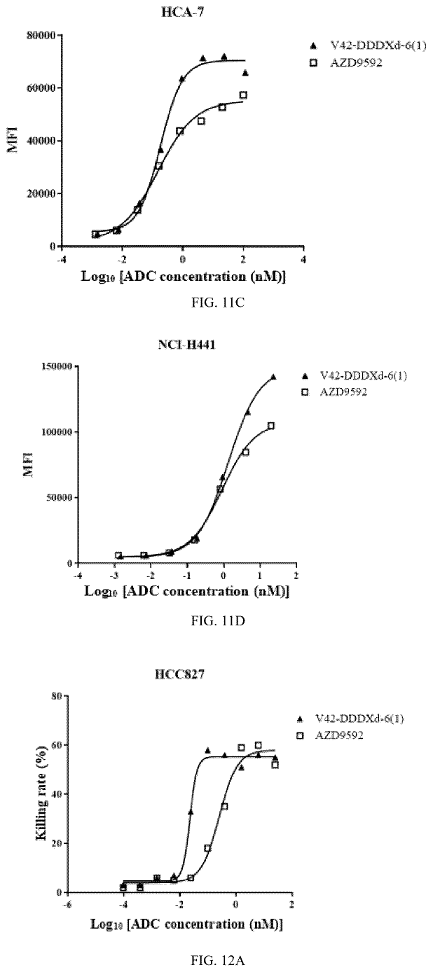

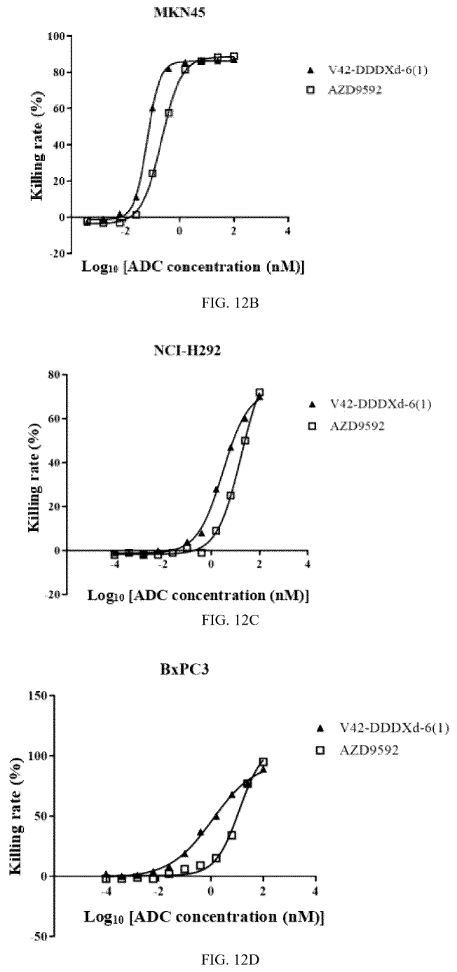

The internalization of the ADCs targeting EGFR and c-Met in cells with different expression levels of EGFR and c-Met was assayed by flow cytometry. HCC827, NCI-H292, HCA-7, and NCI-H441 cells were adjusted to a concentration of 2×10⁶ cells/mL and seeded into a 96-well plate at 50 μL/well. The ADCs targeting EGFR and c-Met serially diluted with FACS buffer (Miltenyi Biotec, Cat. No. 130-091-221) were added at 50 μL/well (for HCC827 and HCA-7 cells, V42-DDDXd-6 (1) at a final concentration of 115.4 nM as the initial concentration, and AZD9592 at a final concentration of 100 nM as the initial concentration, which were 5-fold serially diluted to yield a total of 8 concentrations; for NCI-H292 and NCI-H441 cells, V42-DDDXd-6 (1) at a final concentration of 23.08 nM as the initial concentration, and AZD9592 at a final concentration of 20 nM as the initial concentration, which were 5-fold serially diluted to yield a total of 7 concentrations), and the cells were incubated at 4 °C for 1 h. After the incubation, the 96-well cell culture plate was taken out, the cells were centrifuged at 400 g for 5 min at 4° C., and the supernatant was discarded. A 1:200 (v/v) diluted mouse anti-human IgG antibody (Jackson, 109-005-190) labeled with pHrodo (Thermo, P35358) was added at 100 μL/well. The mixture was mixed thoroughly and then incubated at 4 °C. After incubation for 30 min, the cells were washed, and a cell culture medium was added to each well at 40 μL. The mixture was mixed thoroughly, internalized at 37 °C for 2 h, and then placed in a flow cytometer (Sartorius, iQUE3). The fluorescence reading value in the RLI channel was measured. Data were analyzed. The results are shown in FIGS. 11A to 11D, and the calculated EC₅₀ values are shown in Table 16 below. The results showed that V42-DDDXd-6 (1) could undergo effective internalization in cells with different expression levels of EGFR and c-Met.

TABLE 16
Internalization activity of ADCs targeting EGFR and c-Met

| | HCC827 | | NCI-H292 | | HCA-7 | | NCI-H441 | |
|---|---|---|---|---|---|---|---|---|
| | | EC₅₀ | | EC₅₀ | | EC₅₀ | | EC₅₀ |
| | Top | (nM) | Top | (nM) | Top | (nM) | Top | (nM) |
| V42-DDDXd-6(1) | 410706 | 0.63 | 282365 | 0.77 | 70470 | 0.18 | 148729 | 1.15 |
| AZD9592 | 398527 | 1.82 | 185712 | 9.48 | 55284 | 0.15 | 107829 | 0.91 |

### Example 24: Killing of Tumor Cells by ADCs Targeting EGFR and c-Met

To detect the killing of ADCs targeting EGFR and c-Met on EGFR- and c-MET-positive tumor cells, killing activity assays were performed using natural cell human non-small cell lung cancer cells HCC827 and NCI-H292, human gastric cancer cells MKN45 (low to medium EGFR expression and high c-Met expression, Nanjing Cobioer Biosciences, Cat. No. CBP60488), human colon adenocarcinoma cells HCA-7, and human pancreatic cancer cells BxPC3 (high EGFR expression and low to medium c-Me expression, Procell, Cat. No. CL-0042). The cells in the logarithmic growth phase were taken, the cell density of HCC827, MKN45, NCI-H292, and BxPC3 cells was adjusted to 2×10⁴ cells/mL, and the cell density of HCA-7 cells was adjusted to 4×10⁴ cells/mL. The cells were added to a 96-well plate at 100 μL/well, and adherence culture was performed at 37° C. with 5% CO₂ for 4-6 h. The ADCs targeting EGFR and c-Met were prepared in complete media for the corresponding cells. For HCC827 cells, V42-DDDXd-6 (1) and AZD9592 were 4-fold serially diluted from an initial concentration of 25 nM to yield a total of 10 concentrations. For MKN45, NCI-H292, and BxPC3 cells, V42-DDDXd-6 (1) and AZD9592 were 4-fold serially diluted from an initial concentration of 100 nM to yield a total of 10 or 11 concentrations. For HCA-7 cells, V42-DDDXd-6 (1) and AZD9592 were 4-fold serially diluted from an initial concentration of 25 nM to yield a total of 9 concentrations. The cells after adherence culture were taken out. For the experimental groups, the diluted ADCs targeting EGFR and c-Met were added at 50 μL/well, and for the control group, the corresponding complete medium was added at 50 μL/well. The cells were further cultured for 96 h, 120 h, or 144 h, and then a CellCounting-Lite 2.0 Luminescent Cell Viability Assay kit (Vazyme, Cat. No. DD1101-03) was used for assay. Specifically, the 96-well plate was taken out, a CellCounting-Lite detection solution (Vazyme, Cat. No. DD1101-03) was added to each well at 75 μL, and the mixture was mixed thoroughly by shaking and incubated at room temperature in the dark for 10 min. Then, 170 μL of the solution was pipetted from each well and transferred to an opaque white plate, bubbles were removed, chemiluminescence values were read using a microplate reader (PE, Envision 2105), and the cell killing rate was calculated.

$$Killing rate ( \% ) = \frac{\begin{matrix}( Luminescence value of the control group - \\ Luminescence value of the experimental group )\end{matrix}}{Luminescence value of the control group} \times 100 \%$$

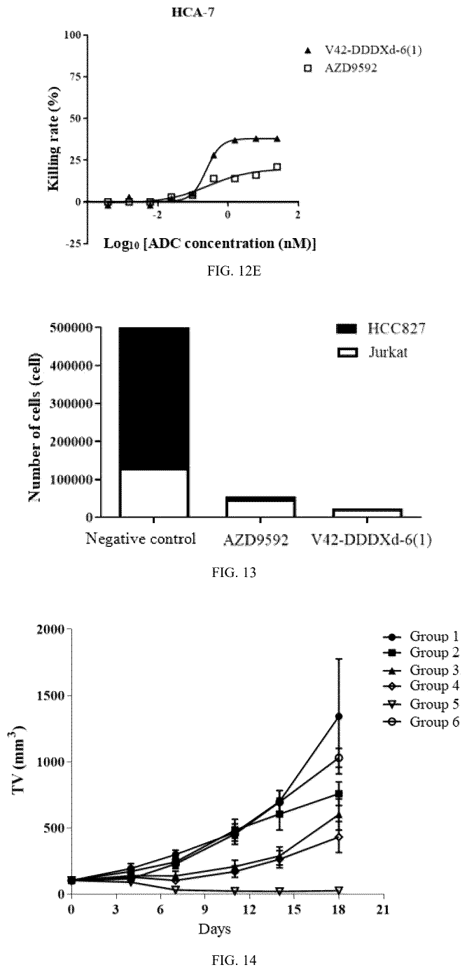

Data were analyzed. The results are shown in FIGS. 12A to 12E, and the calculated EC₅₀ values are shown in Table 17 below. The results showed that the killing effect of V42-DDDXd-6 (1) on tumor cells was superior to that of AZD9592.

TABLE 17
Killing effect of ADCs targeting EGFR and c-Met on tumor cells

| | HCC827 | | MKN45 | | NCI-H292 | | BxPC3 | | HCA-7 | |
|---|---|---|---|---|---|---|---|---|---|---|
| | Maxi- | | Maxi- | | Maxi- | | Maxi- | | Maxi- | |
| | mum | | mum | | mum | | mum | | mum | |
| | killing | | killing | | killing | | killing | | killing | |
| | rate | EC₅₀ | rate | EC₅₀ | rate | EC₅₀ | rate | EC₅₀ | rate | EC₅₀ |
| | (%) | (nM) | (%) | (nM) | (%) | (nM) | (%) | (nM) | (%) | (nM) |
| V42- | 55.25 | 0.023 | 86.24 | 0.062 | 72.76 | 2.96 | 95.17 | 1.13 | 37.95 | 0.24 |
| DDDXd-6(1) | | | | | | | | | | |
| AZD9592 | 57.96 | 0.25 | 88.64 | 0.21 | 87.75 | 17.08 | 109.7 | 12.13 | 19.99 | 0.27 |

### Example 25: Bystander Effect of ADCs Targeting EGFR and c-Met on Tumor Cells

To detect the bystander effect of ADCs targeting EGFR and c-Met, an FACS-based assay was performed using HCC827 as the positive cell and Jurkat as the negative cell. HCC827 and Jurkat cells in the logarithmic growth phase were taken. The viable cell density of HCC827 was adjusted to 2×10⁴ cells/mL, and the cells were added to a 6-well plate at 1 mL/well and then incubated overnight in a cell incubator at 37 °C with 5% CO₂. The ADCs were pre-diluted to 1.6 nM (final concentration) and added to the cell plate at 2 mL/well. The viable cell density of Jurkat was adjusted to 1×10⁴ cells/mL, and the cells were added to the corresponding cell plate at 1 mL/well. After incubation for 5 days, the cells in the 6-well plate were taken out, digested with trypsin, and counted. The cells in different wells were collected and adjusted to a density of 1×10⁶ cells/mL with FACS buffer, and then the cells were added to a 96-well V-bottom plate at 100 μL/well. A cell viability identifying dye Fixable Viability Stain 780 (BD, Cat. No. 565388) was added to the 96-well plate. The cells were incubated at room temperature for 15 min, and then washed twice. Subsequently, an EGFR direct-labeled antibody (abcam, Cat. No. 130738) was added, and the cells were incubated at 4 °C for 40 min. After incubation, the cells were washed twice and resuspended in an FACS buffer solution, and the signals were detected using a flow cytometer (Invitrogen, model: Attune®NxT).

Data were analyzed. The results are shown in FIG. 13, which demonstrated that V42-DDDXd-6 (1) had a bystander effect.

### Example 26: Pharmacodynamic Evaluation of ADCs Targeting EGFR and c-Met in NCI-H292 Human Lung Cancer Cell Nude Mouse Xenograft Tumor Model

SPF-grade female BALB/c nude mice (source: Laboratory Animal Center, Hangzhou Medical College) were each inoculated subcutaneously at the right side axilla with 5×10⁶ NCI-H292 human lung cancer cells. When the mean tumor volume reached about 100 mm³, the animals were divided into 6 groups with 5 animals per group. The specific grouping and administration regimen are shown in Table 18:

TABLE 18
Grouping and administration regimens

| | | ADC or antibody dose |
|---|---|---|
| | | (cytotoxic drug dose) |
| Group | Drug | mg/kg |
| 1 | Vehicle control | N/A |
| 2 | AZD9592 | 1.000 (0.016) |
| 3 | AZD9592 | 3.000 (0.048) |
| 4 | V42-DDDXd-6(1) | 0.721 (0.016) |
| 5 | V42-DDDXd-6(1) | 2.164 (0.048) |
| 6 | V42 | 2.065 |

The day of grouping was defined as day 0, and tail vein administration was performed once on day 1 after grouping. The tumor volume was measured twice every week, and meanwhile, the mice were weighed, and the data were recorded. The general behavior of the mice was observed and recorded every day. After the experiment was completed, the tumors were removed, weighed, and photographed.

The detection parameters and the calculation formulas are as follows:
Tumor volume TV (mm³)=½×(a×b²), where a represents the long diameter of the tumor, and b represents the short diameter of the tumor.
Relative tumor volume RTV=TVₜ/TV₀, where TV₀ represents the tumor volume on day 0, and TVₜ represents the tumor volume at each measurement;
Relative tumor proliferation rate T/C (%)=(T_{RTV}/C_{RTV})×100%, where T_{RTV} represents RTV of the treatment group, and C_{RTV} represents RTV of the vehicle control group.
Tumor growth inhibition rate TGI (%)=(1−TW/TW₀)×100%, where TW represents the tumor weight of the treatment group, and TW₀ represents the tumor weight of the vehicle control group.
Weight change rate WCR (%)=(Wtₜ−Wt₀)/Wt₀×100%, where Wt₀ represents the body weight of the mouse on day 0, and Wtₜ represents the body weight of the mouse at each measurement.

The results of all detection parameters on day 18 are shown in Table 19 below and FIG. 14. No significant toxicity was observed in all groups. The low and high doses of V42-DDDXd-6 (1) all exhibited inhibitory activity against mouse NCI-H292 human lung cancer xenograft tumor. At the same cytotoxic drug dose, the efficacy of V42-DDDXd-6 (1) was superior to that of AZD9592.

TABLE 19
Effect of ADCs targeting EGFR and c-Met
on parameters in NCI-H292 human lung cancer
cell nude mouse subcutaneous xenograft tumor model

| | TV (mm³) | RTV | | | Weight change |
|---|---|---|---|---|---|
| | (Mean ± | (Mean ± | | | rate (%) |
| Group | SD) | SD) | T/C | TGI | (Mean ± SD) |
| 1 | 1341 ± 971 | 14.9 ± 13.2 | N/A | N/A | 5.7 ± 4.7 |
| 2 | 757 ± 200 | 7.7 ± 2.2 | 51.7% | 25.6% | 0.1 ± 4.4 |
| 3 | 600 ± 263 | 5.6 ± 0.9 | 37.6% | 50.7% | 4.7 ± 1.8 |
| 4 | 430 ± 263 | 4.0 ± 2.1 | 26.8% | 66.3% | 9.0 ± 1.8 |
| 5 | 25 ± 34 | 0.3 ± 0.3* | 2.0% | 98.1% | 3.0 ± 6.7 |
| 6 | 1028 ± 160 | 10.1 ± 1.5 | 67.8% | 17.9% | 6.5 ± 7.9 |

Note:
Compared with the vehicle control group (group 1),
*P < 0.05; N/A denotes not applicable.

### Example 27: Pharmacodynamic Evaluation of ADCs Targeting EGFR and c-Met in HCA-7 Human Colon Cancer Cell Nude Mouse Xenograft Tumor Model

SPF-grade female BALB/c nude mice (source: Laboratory Animal Center, Hangzhou Medical College) were each inoculated subcutaneously at the right side axilla with 2×10⁶ HCA-7 human colon cancer cells. When the mean tumor volume reached about 150 mm³, the animals were divided into 6 groups with 5 animals per group. The specific grouping and administration regimen are shown in Table 20:

TABLE 20
Mouse groups and doses

| | | ADC or antibody dose |
|---|---|---|
| | | (cytotoxic drug dose) |
| Group | Drug | mg/kg |
| 1 | Vehicle control | N/A |
| 2 | AZD9592 | 1.000 (0.016) × 2 times + |
| | | 2.000 (0.032) × 2 times |
| 3 | AZD9592 | 3.000 (0.048) × 2 times + |
| | | 6.000 (0.096) × 2 times |
| 4 | V42-DDDXd-6(2) | 0.727 (0.016) × 2 times + |
| | | 1.448 (0.032) × 2 times |
| 5 | V42-DDDXd-6(2) | 2.182 (0.048) × 2 times + |
| | | 4.343 (0.096) × 2 times |
| 6 | V42 | 2.082 × 2 times + 4.145 × |
| | | 2 times |

The day of grouping was defined as day 0, and tail vein administration was performed on day 1 after grouping and performed twice a week for a total of 4 administrations. The tumor volume was measured twice every week, and meanwhile, the mice were weighed, and the data were recorded. The general behavior of the mice was observed and recorded every day. After the experiment was completed, the tumors were removed, weighed, and photographed.

The detection parameters and the calculation formulas are as follows:
Tumor volume TV (mm³)=½×(a×b²), where a represents the long diameter of the tumor, and b represents the short diameter of the tumor.
Relative tumor volume RTV=TVₜ/TV₀, where TV₀ represents the tumor volume on day 0, and TVₜ represents the tumor volume at each measurement;
Relative tumor proliferation rate T/C (%)=(T_{RTV}/C_{RTV})×100%, where T_{RTV} represents RTV of the treatment group, and C_{RTV} represents RTV of the vehicle control group.
Tumor growth inhibition rate TGI (%)=(1−TW/TW₀)×100%, where TW represents the tumor weight of the treatment group, and TW₀ represents the tumor weight of the vehicle control group.
Weight change rate WCR (%)=(Wtₜ−Wt₀)/Wt₀×100%, where Wt₀ represents the body weight of the mouse on day 0, and Wtₜ represents the body weight of the mouse at each measurement.

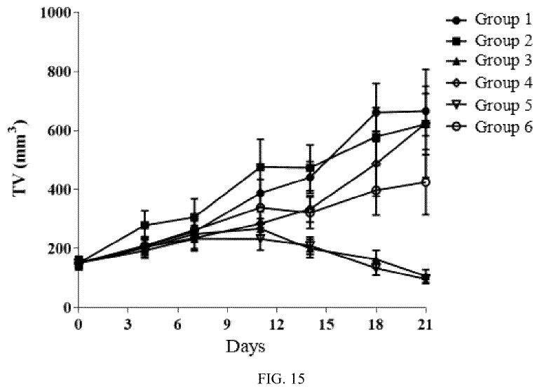

The results of all detection parameters on day 21 are shown in Table 21 below and FIG. 15. No significant toxicity was observed in all groups. The V42-DDDXd-6 (2) at high dose had inhibitory activity against mouse HCA-7 human colon cancer xenograft tumor.

TABLE 21
Effect of ADC-EGFR cMet on parameters in HCA-7 human
colon cancer cell nude mouse subcutaneous xenograft tumor model

| | TV (mm³) | RTV | | | Weight change rate |
|---|---|---|---|---|---|
| Group | (Mean ± SD) | (Mean ± SD) | T/C | TGI | (%) (Mean ± SD) |
| 1 | 666 ± 88 | 4.67 ± 1.79 | N/A | N/A | 7.5 ± 3.3 |
| 2 | 621 ± 232 | 4.1 ± 0.31 | 87.8% | 8.4% | 4.9 ± 5.3 |
| 3 | 106 ± 50 | 0.69 ± 0.30 | 14.8% | 86.1% | 5.4 ± 2.9 |
| 4 | 624 ± 410 | 4.27 ± 2.45 | 91.4% | 17.6% | 3.1 ± 2.6 |
| 5 | 96 ± 33 | 0.63 ± 0.16* | 13.5% | 82.2% | 3.9 ± 2.9 |
| 6 | 425 ± 248 | 2.77 ± 1.47 | 59.3% | 44.6% | 5.1 ± 4.9 |

Note:
Compared with the vehicle control group,
*P < 0.05; N/A denotes not applicable.

The sequence information of the present disclosure is summarized in Table S4 below.

TABLE S4
Sequence information
Description
Sequence (SEQ ID NO:)

CDR1 of 1B-3B11
SVA (SEQ ID NO: 1)
CDR2 of 1B-3B11
VINPGGDNTDYTYAVKG (SEQ ID NO: 2)
CDR3 of 1B-3B11
GRTFGGTWMDRNKWKY (SEQ ID NO: 3)
CDR1 of 1B-1B6, 1B-1B6-V1, 1B-1B6-V2, and 1B-1B6-V3
SGCMG (SEQ ID NO: 4)
CDR2 of 1B-1B6, 1B-1B6-V1, 1B-1B6-V2, and 1B-1B6-V3
TIYTRNDFTYYSDX₁VKG (SEQ ID NO: 5, wherein X₁ is selected from S and T)
CDR2 of 1B-1B6, 1B-1B6-V1, and 1B-1B6-V2
TIYTRNDFTYYSDX₁VKG (SEQ ID NO: 5, wherein X₁ is S), i.e.,
TIYTRNDFTYYSDSVKG
CDR2 of 1B-1B6-V3
TIYTRNDFTYYSDX₁VKG (SEQ ID NO: 5, wherein X₁ is T), i.e.,
TIYTRNDFTYYSDTVKG
CDR3 of 1B-1B6, 1B-1B6-V1, 1B-1B6-V2, and 1B-1B6-V3
GSPFRYAGAWNVCGLKEEDYNY (SEQ ID NO: 6)
CDR1 of 1B-1C7, 1B-1C7-V1, and 1B-1C7-V2
ANCVG (SEQ ID NO: 7)
CDR2 of 1B-1C7, 1B-1C7-V1, and 1B-1C7-V2
AINTGGETATYADFVKG (SEQ ID NO: 8)
CDR3 of 1B-1C7, 1B-1C7-V1, and 1B-1C7-V2
VGTRKYWDECSLSQHAYKN (SEQ ID NO: 9)
CDR1 of 1B-1A8
SYAMD (SEQ ID NO: 10)
CDR2 of 1B-1A8
GINSAGSPSYEASVKG (SEQ ID NO: 11)
CDR3 of 1B-1A8
CRDFRSPYCR (SEQ ID NO: 12)
CDR1 of 1B-1B2
MG (SEQ ID NO: 13)
CDR2 of 1B-1B2
SISSDGNSDYLDSVKG (SEQ ID NO: 14)
CDR3 of 1B-1B2
NGAGHLVLRPLCGSNAVDA (SEQ ID NO: 15)
CDR1 of 3B-1C7
GTTMG (SEQ ID NO: 16)
CDR2 of 3B-1C7
DISSDGNTYYSDSVKG (SEQ ID NO: 17)
CDR3 of 3B-1C7
AEFRLLLRPLCRODDYGY (SEQ ID NO: 18)
CDR1 of 4&5B-2F01
YKYLA (SEQ ID NO: 19)
CDR2 of 4&5B-2F01
GLNTGGGSAYYADSVKG (SEQ ID NO: 20)
CDR3 of 4&5B-2F01
RRDYIHMTNIQALGLRPQWFSD (SEQ ID NO: 21)
CDR1 of 4&5C-12B04, 12B04-V1, and 12B04-V2
SNFMA (SEQ ID NO: 22)
CDR2 of 4&5C-12B04, 12B04-V1, and 12B04-V2
TITTGAGNTAYADSVKG (SEQ ID NO: 23)
CDR3 of 4&5C-12B04, 12B04-V1, and 12B04-V2
GRGGFWRPAEYDF (SEQ ID NO: 24)
V_{H}H of 1B-3B11
QVQLVESGGGSVQAGGSLRLSCVVSGYTYSSVAWFRQTPGKEREGVAVINPGGDNTDYTYAVKGR
FTISRDSAKSTVFLQMNNLESEDTAMYYCAAGRTFGGTWMDRNKWKYWGQGTLVTVSS (SEQ ID
NO: 25)
V_{H}H of 1B-1B6
QVQLVESGGDSVQAGGSLRLSCAASAHAYSSGCMGWFRQAPGKGREGVATIYTRNDFTYYSDSVK
GRFTISRDDAKNTVYLQVNSLKAEDTAMYYCAAGSPFRYAGAWNVCGLKEEDYNYWGQGTLVT
VSS (SEQ ID NO: 26)
V_{H}H of 1B-1C7
QVQLVESGGGLVQPGGSLRLSCAASGYSYSANCVGWFYQAPGKEREGVAAINTGGETATYADFVK
GRFTISQDYAKNTVYLQMNSLKSEDTGMYYCAAVGTRKYWDECSLSQHAYKNWGQGTLVTVSS
(SEQ ID NO: 27)
V_{H}H of 1B-1A8
EVQLVESGGGLVQPGGSLRLSCVASGFTFKSYAMDWVRQAPGKGLEWIAGINSAGSPSYEASVKG
RFTISRDNAENTLYLQLNSLKTEDTAMYYCTKCRDFRSPYCRPGQGTLVTVSS (SEQ ID NO: 28)
V_{H}H of 1B-1B2
EVQLVESGGGSVQAGGSLRLSCTASGFTFDMGWYRQAPGDECELVSSISSDGNSDYLDSVKGRFTI
SQDNAKNTVYLQMNSLKPEDTAVYYCAANGAGHLVLRPLCGSNAVDAWGQGTLVTVSS (SEQ ID
NO: 29)
V_{H}H of 3B-1C7
QVQLVESGGDSVQAGGSLRLSCIASGFTFDGTTMGWYRQAPGDECELVSDISSDGNTYYSDSVKG
RFTISQGNAKNTVYLQMNSLTPEDTAVYYCAAAEFRLLLRPLCRQDDYGYWGQGTLVTVSS (SEQ
ID NO: 30)
V_{H}H of 4&5B-2F01
QVQLQESGGGLVQPGGSLRLSCAASGTTGDYKYLAWFRQGPGKEREGIAGLNTGGGSAYYADSVK
GRFTISQDNVKRLLYLQLNNLKPEDTAMYYCAARRDYIHMTNIQALGLRPQWFSDWGQGTQVTV
SS (SEQ ID NO: 31)
V_{H}H of 4&5C-12B04
EVQLVESGGGSVQAQGSLRLSCTVSGSTYSSNFMAWFRQAPGKEREWVATITTGAGNTAYADSVK
GRFTISEDNAKRTAYLQMNSLKPEDTAVYYCAAGRGGFWRPAEYDFWGQGTQVTVSS (SEQ ID
NO: 32)
V_{H}H of 1B-1B6-V1
QVQLLESGGGSVQAGGSLRLSCAASGFTFSSGCMGWFRQAPGKGREGVATIYTRNDFTYYSDSVK
GRFTISRDNSKNTVYLQMNSLRAEDTAVYYCAAGSPFRYAGAWNVCGLKEEDYNYWGQGTLVTV
SS (SEQ ID NO: 33)
V_{H}H of 1B-1B6-V2
QVQLLESGGGSVQAGGSLRLSCAASGHTYSSGCMGWFRQAPGKGREGVATIYTRNDFTYYSDSVK
GRFTISRDNSKNTVYLQMNSLRAEDTAVYYCAAGSPFRYAGAWNVCGLKEEDYNYWGQGTLVTV
SS (SEQ ID NO: 34)
V_{H}H of 1B-1B6-V3
QVQLLESGGGSVQAGGSLRLSCAASGHTYSSGCMGWFRQAPGKGREGVATIYTRNDFTYYSDTVK
GRFTISRDNSKNTVYLQMNSLRAEDTAVYYCAAGSPFRYAGAWNVCGLKEEDYNYWGQGTLVTV
SS (SEQ ID NO: 35)
V_{H}H of 1B-1C7-V1
QVQLVESGGGSVQPGGSLRLSCAASGYSYSANCVGWFRQAPGKGREGVSAINTGGETATYADFVK
GRFTISRDNAKNTVYLQMNSLRAEDTAVYYCAAVGTRKYWDECSLSQHAYKNWGQGTLVTVSS
(SEQ ID NO: 36)
V_{H}H of 1B-1C7-V2
QVQLVESGGGSVQPGGSLRLSCAASGFTFSANCVGWFRQAPGKGREGVSAINTGGETATYADFVK
GRFTISRDNAKNTVYLQMNSLRAEDTAVYYCAAVGTRKYWDECSLSQHAYKNWGQGTLVTVSS
(SEQ ID NO: 37)
12B04-V1
EVQLVESGGGSVQAGGSLRLSCAVSGFTVSSNFMAWFRQAPGKGREWVSTITTGAGNTAYADSVK
GRFTISEDNSKNTLYLQMNSLRAEDTAVYYCAAGRGGFWRPAEYDFWGQGTTVTVSS (SEQ ID
NO: 38)
12B04-V2
EVQLVESGGGSVQAGGSLRLSCAVSGFTVSSNFMAWFRQAPGKGREWVSTITTGAGNTAYADSVK
GRFTISEDNSKNTAYLQMNSLRAEDTAVYYCAAGRGGFWRPAEYDFWGQGTTVTVSS (SEQ ID
NO: 39)
V_{H}H of camel and humanized 1B-1B6
QVQLX₂ESGGX₃SVQAGGSLRLSCAASX₄X₅X₆X₇SSGCMGWFRQAPGKGREGVATIYTRNDFTYYS
DX₈VKGRFTISRDX₉X₁₀KNTVYLQX₁₁NSLX₁₂AEDTAX₁₃YYCAAGSPFRYAGAWNVCGLKEEDYNY
WGQGTLVTVSS (SEQ ID NO: 40, wherein X₂ is selected from V and L, X₃ is selected from D and
G, X₄ is selected from A and G, X₅ is selected from H and F, X₆ is selected from A and T, X₇
is selected from Y and F, X₈ is selected from S and T, X₉ is selected from D and N, X₁₀ is
selected from A and S, X₁₁ is selected from V and M, X₁₂ is selected from K and R, and X₁₃ is
selected from M and V)
V_{H}H of camel and humanized 1B-1C7
QVQLVESGGGX₁₄VQPGGSLRLSCAASGX₁₅X₁₆X₁₇SANCVGWFX₁₈QAPGKX₁₉REGVX₂₀AINTGGET
ATYADFVKGRFTISX₂₁DX₂₂AKNTVYLQMNSLX₂₃X₂₄EDTX₂₅X₂₆YYCAAVGTRKYWDECSLSQHAY
KNWGQGTLVTVSS (SEQ ID NO: 41, wherein X₁₄ is selected from L and S, X₁₅ is selected from Y
and F, X₁₆ is selected from S and T, X₁₇ is selected from Y and F, X₁₈ is selected from Y and
R, X₁₉ is selected from E and G, X₂₀ is selected from A and S, X₂₁ is selected from Q and R,
X₂₂ is selected from Y and N, X₂₃ is selected from K and R, X₂₄ is selected from S and A, X₂₅
is selected from G and A, and X₂₆ is selected from M and V)
V_{H}H of camel and humanized 4&5C-12B04
EVQLVESGGGSVQAX₂₇GSLRLSCX₂₈VSGX₂₉TX₃₀SSNFMAWFRQAPGKX₃₁REWVX₃₂TITTGAGNT
AYADSVKGRFTISEDNX₃₃KX₃₄TX₃₅YLQMNSLX₃₆X₃₇EDTAVYYCAAGRGGFWRPAEYDFWGQGTX₃₈
VTVSS (SEQ ID NO: 42, wherein X₂₇ is selected from Q and G, X₂₈ is selected from T and A,
X₂₉ is selected from S and F, X₃₀ is selected from Y and V, X₃₁ is selected from E and G, X₃₂
is selected from A and S, X₃₃ is selected from A and S, X₃₄ is selected from R and N, X₃₅ is
selected from A and L, X₃₆ is selected from K and R, X₃₇ is selected from P and A, and X₃₈ is
selected from Q and T)
1B-3B11-Fc
QVQLVESGGGSVQAGGSLRLSCVVSGYTYSSVAWFRQTPGKEREGVAVINPGGDNTDYTYAVKGR
FTISRDSAKSTVFLQMNNLESEDTAMYYCAAGRTFGGTWMDRNKWKYWGQGTLVTVSSEPKIPQ
PQPKPQPQPQPQPKPQPKPEPECTCPKCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK (SEQ ID NO: 43)
1B-1B6-Fc
QVQLVESGGDSVQAGGSLRLSCAASAHAYSSGCMGWFRQAPGKGREGVATIYTRNDFTYYSDSVK
GRFTISRDDAKNTVYLQVNSLKAEDTAMYYCAAGSPFRYAGAWNVCGLKEEDYNYWGQGTLVT
VSSEPKIPQPQPKPQPQPQPQPKPQPKPEPECTCPKCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK (SEQ ID NO:
44)
1B-1C7-Fc
QVQLVESGGGLVQPGGSLRLSCAASGYSYSANCVGWFYQAPGKEREGVAAINTGGETATYADFVK
GRFTISQDYAKNTVYLQMNSLKSEDTGMYYCAAVGTRKYWDECSLSQHAYKNWGQGTLVTVSSE
PKIPQPQPKPQPQPQPQPKPQPKPEPECTCPKCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK (SEQ ID NO: 45)
1B-1A8-Fc
EVQLVESGGGLVQPGGSLRLSCVASGFTFKSYAMDWVRQAPGKGLEWIAGINSAGSPSYEASVKG
RFTISRDNAENTLYLQLNSLKTEDTAMYYCTKCRDFRSPYCRPGQGTLVTVSSEPKIPQPQPKPQPQ
PQPQPKPQPKPEPECTCPKCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK (SEQ ID NO: 46)
1B-1B2-Fc
EVQLVESGGGSVQAGGSLRLSCTASGFTFDMGWYRQAPGDECELVSSISSDGNSDYLDSVKGRFTI
SQDNAKNTVYLQMNSLKPEDTAVYYCAANGAGHLVLRPLCGSNAVDAWGQGTLVTVSSEPKIPQP
QPKPQPQPQPQPKPQPKPEPECTCPKCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK (SEQ ID NO: 47)
3B-1C7-Fc
QVQLVESGGDSVQAGGSLRLSCIASGFTFDGTTMGWYRQAPGDECELVSDISSDGNTYYSDSVKG
RFTISQGNAKNTVYLQMNSLTPEDTAVYYCAAAEFRLLLRPLCRQDDYGYWGQGTLVTVSSEPKIP
QPQPKPQPQPQPQPKPQPKPEPECTCPKCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK (SEQ ID NO: 48)
4&5B-2F01-Fc
QVQLQESGGGLVQPGGSLRLSCAASGTTGDYKYLAWFRQGPGKEREGIAGLNTGGGSAYYADSVK
GRFTISQDNVKRLLYLQLNNLKPEDTAMYYCAARRDYIHMTNIQALGLRPQWFSDWGQGTQVTV
SSEPKSSDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGK (SEQ ID NO: 49)
4&5C-12B04-Fc
EVQLVESGGGSVQAQGSLRLSCTVSGSTYSSNFMAWFRQAPGKEREWVATITTGAGNTAYADSVK
GRFTISEDNAKRTAYLQMNSLKPEDTAVYYCAAGRGGFWRPAEYDFWGQGTQVTVSSEPKSSDKT
HTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGN
VFSCSVMHEALHNHYTQKSLSLSPGK (SEQ ID NO: 50)
1B-1B6-V1
QVQLLESGGGSVQAGGSLRLSCAASGFTFSSGCMGWFRQAPGKGREGVATIYTRNDFTYYSDSVK
GRFTISRDNSKNTVYLQMNSLRAEDTAVYYCAAGSPFRYAGAWNVCGLKEEDYNYWGQGTLVTV
SSGEPKSSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK (SEQ ID NO: 51)
caggtgcagctgctggagagcggcggcggaagcgtgcaggctggaggatccctgcggctgagctgtgctgcctccggcttcaccttcagct
ccggctgcatgggctggttcaggcaggcccctggcaagggccgggagggagttgctaccatctacacccggaatgacttcacctattactc
cgacagcgtgaagggcaggttcaccatcagccgggacaactccaagaacaccgtgtatctgcagatgaatagcctgcgggccgaggacacc
gccgtgtactattgcgctgccggcagccccttcaggtacgctggcgcttggaatgtgtgtggcctgaaggaggaggattacaattattggg
gccagggcaccctggtgaccgtgagcagcggagagccaaagtccagcgataagacccacacctgccccccttgccctgctcccgagctgct
gggaggccctagcgtgttcctgttcccccctaagcctaaggacaccctgatgatcagccggacccccgaggtgacctgtgtggtggtggat
gtgagccacgaggatcctgaggtgaagttcaactggtatgtggacggcgtggaggtgcacaacgccaagaccaagcctagggaggagcagt
ataactccacctatagggtggtgagcgtgctgaccgtgctgcaccaggattggctgaatggcaaggagtacaagtgcaaggtgagcaataa
ggctctgcctgctcctatcgagaagaccatctccaaggctaagggccagcccagggagcctcaggtgtacaccctgcctcccagcagggac
gagctgaccaagaatcaggtgagcctgacctgcctggtgaagggcttctacccctccgatatcgctgtggagtgggagagcaacggccagc
ctgagaacaactacaagaccaccccccccgtgctggattccgatggctccttcttcctgtacagcaagctgaccgtggataagagccggtg
gcagcagggcaacgtgttcagctgctccgtgatgcacgaggccctgcacaaccactacacccagaagagcctgagcctgagccctggcaag
(SEQ ID NO: 52)
1B-1B6-V2
QVQLLESGGGSVQAGGSLRLSCAASGHTYSSGCMGWFRQAPGKGREGVATIYTRNDFTYYSDSVK
GRFTISRDNSKNTVYLQMNSLRAEDTAVYYCAAGSPFRYAGAWNVCGLKEEDYNYWGQGTLVTV
SSGEPKSSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK (SEQ ID NO: 53)
caggtgcagctgctggagtccggcggcggatccgtgcaggctggaggatccctgcggctgagctgcgctgcttccggccacacctactcct
ccggctgcatgggctggttcaggcaggctcctggcaagggccgggagggagtggctaccatctataccaggaacgacttcacctactattc
cgattccgtgaagggcaggttcaccatcagcagggataactccaagaacaccgtgtatctgcagatgaatagcctgcgggccgaggatacc
gccgtgtactactgcgccgccggcagccccttccggtacgctggagcttggaacgtgtgcggcctgaaggaggaggactacaactattggg
gccagggcaccctggtgaccgtgagctccggagagccaaagtccagcgataagacccacacctgccccccttgccctgctcccgagctgct
gggaggccctagcgtgttcctgttcccccctaagcctaaggacaccctgatgatcagccggacccccgaggtgacctgtgtggtggtggat
gtgagccacgaggatcctgaggtgaagttcaactggtatgtggacggcgtggaggtgcacaacgccaagaccaagcctagggaggagcagt
ataactccacctatagggtggtgagcgtgctgaccgtgctgcaccaggattggctgaatggcaaggagtacaagtgcaaggtgagcaataa
ggctctgcctgctcctatcgagaagaccatctccaaggctaagggccagcccagggagcctcaggtgtacaccctgcctcccagcagggac
gagctgaccaagaatcaggtgagcctgacctgcctggtgaagggcttctacccctccgatatcgctgtggagtgggagagcaacggccagc
ctgagaacaactacaagaccaccccccccgtgctggattccgatggctccttcttcctgtacagcaagctgaccgtggataagagccggtg
gcagcagggcaacgtgttcagctgctccgtgatgcacgaggccctgcacaaccactacacccagaagagcctgagcctgagccctggcaag
(SEQ ID NO: 54)
1B-1B6-V3
QVQLLESGGGSVQAGGSLRLSCAASGHTYSSGCMGWFRQAPGKGREGVATIYTRNDFTYYSDTVK
GRFTISRDNSKNTVYLQMNSLRAEDTAVYYCAAGSPFRYAGAWNVCGLKEEDYNYWGQGTLVTV
SSGEPKSSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK (SEQ ID NO: 55)
caggtgcagctgctggagagcggcggcggaagcgtgcaggctggaggaagcctgaggctgagctgcgccgccagcggacacacctacagct
ccggctgtatgggctggttcaggcaggcccccggcaagggacgggagggagttgctaccatctacacccggaatgacttcacctactattc
cgataccgtgaagggcaggttcaccatcagcagggacaactccaagaataccgtgtacctgcagatgaactccctgcgggccgaggacacc
gccgtgtattactgtgctgctggctcccccttcaggtatgccggcgcttggaatgtgtgcggcctgaaggaggaggactataattattggg
gccagggcaccctggtgaccgtgagcagcggagagccaaagtccagcgataagacccacacctgccccccttgccctgctcccgagctgct
gggaggccctagcgtgttcctgttcccccctaagcctaaggacaccctgatgatcagccggacccccgaggtgacctgtgtggtggtggat
gtgagccacgaggatcctgaggtgaagttcaactggtatgtggacggcgtggaggtgcacaacgccaagaccaagcctagggaggagcagt
ataactccacctatagggtggtgagcgtgctgaccgtgctgcaccaggattggctgaatggcaaggagtacaagtgcaaggtgagcaataa
ggctctgcctgctcctatcgagaagaccatctccaaggctaagggccagcccagggagcctcaggtgtacaccctgcctcccagcagggac
gagctgaccaagaatcaggtgagcctgacctgcctggtgaagggcttctacccctccgatatcgctgtggagtgggagagcaacggccagc
ctgagaacaactacaagaccaccccccccgtgctggattccgatggctccttcttcctgtacagcaagctgaccgtggataagagccggtg
gcagcagggcaacgtgttcagctgctccgtgatgcacgaggccctgcacaaccactacacccagaagagcctgagcctgagccctggcaag
(SEQ ID NO: 56)
1B-1C7-V1
QVQLVESGGGSVQPGGSLRLSCAASGYSYSANCVGWFRQAPGKGREGVSAINTGGETATYADFVK
GRFTISRDNAKNTVYLQMNSLRAEDTAVYYCAAVGTRKYWDECSLSQHAYKNWGQGTLVTVSSG
EPKSSDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK (SEQ ID NO: 57)
caggtgcagctggtggagtccggcggcggaagcgtgcagcctggaggaagcctgaggctgtcctgcgctgcctccggctatagctacagcg
ctaactgcgtgggctggttccggcaggctcctggcaagggccgggagggagtgtccgctatcaacaccggcggcgagaccgctacctacgc
cgacttcgtgaagggccggttcaccatctcccgggataacgctaagaataccgtgtacctgcagatgaatagcctgcgggctgaggatacc
gccgtgtattattgtgctgccgtgggcacccggaagtactgggacgagtgcagcctgagccagcacgcctacaagaattggggccagggca
ccctggtgaccgtgtccagcggagagccaaagtccagcgataagacccacacctgccccccttgccctgctcccgagctgctgggaggccc
tagcgtgttcctgttcccccctaagcctaaggacaccctgatgatcagccggacccccgaggtgacctgtgtggtggtggatgtgagccac
gaggatcctgaggtgaagttcaactggtatgtggacggcgtggaggtgcacaacgccaagaccaagcctagggaggagcagtataactcca
cctatagggtggtgagcgtgctgaccgtgctgcaccaggattggctgaatggcaaggagtacaagtgcaaggtgagcaataaggctctgcc
tgctcctatcgagaagaccatctccaaggctaagggccagcccagggagcctcaggtgtacaccctgcctcccagcagggacgagctgacc
aagaatcaggtgagcctgacctgcctggtgaagggcttctacccctccgatatcgctgtggagtgggagagcaacggccagcctgagaaca
actacaagaccaccccccccgtgctggattccgatggctccttcttcctgtacagcaagctgaccgtggataagagccggtggcagcaggg
caacgtgttcagctgctccgtgatgcacgaggccctgcacaaccactacacccagaagagcctgagcctgagccctggcaag
(SEQ ID NO: 58)
1B-1C7-V2
QVQLVESGGGSVQPGGSLRLSCAASGFTFSANCVGWFRQAPGKGREGVSAINTGGETATYADFVK
GRFTISRDNAKNTVYLQMNSLRAEDTAVYYCAAVGTRKYWDECSLSQHAYKNWGQGTLVTVSSG
EPKSSDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK (SEQ ID NO: 59)
caggtgcagctggtggagtccggcggcggatccgtgcagcctggaggatccctgaggctgagctgcgccgccagcggattcaccttcagcg
ccaactgcgtgggctggttccggcaggctcctggcaagggccgggagggagtgagcgctatcaacaccggcggcgagaccgccacctatgc
cgacttcgtgaagggccggttcaccatctccagggataatgctaagaacaccgtgtacctgcagatgaatagcctgagggccgaggatacc
gccgtgtattactgtgccgccgtgggcaccaggaagtattgggacgagtgctccctgtcccagcacgcttacaagaactggggccagggca
ccctggtgaccgtgagcagcggagagccaaagtccagcgataagacccacacctgccccccttgccctgctcccgagctgctgggaggccc
tagcgtgttcctgttcccccctaagcctaaggacaccctgatgatcagccggacccccgaggtgacctgtgtggtggtggatgtgagccac
gaggatcctgaggtgaagttcaactggtatgtggacggcgtggaggtgcacaacgccaagaccaagcctagggaggagcagtataactcca
cctatagggtggtgagcgtgctgaccgtgctgcaccaggattggctgaatggcaaggagtacaagtgcaaggtgagcaataaggctctgcc
tgctcctatcgagaagaccatctccaaggctaagggccagcccagggagcctcaggtgtacaccctgcctcccagcagggacgagctgacc
aagaatcaggtgagcctgacctgcctggtgaagggcttctacccctccgatatcgctgtggagtgggagagcaacggccagcctgagaaca
actacaagaccaccccccccgtgctggattccgatggctccttcttcctgtacagcaagctgaccgtggataagagccggtggcagcaggg
caacgtgttcagctgctccgtgatgcacgaggccctgcacaaccactacacccagaagagcctgagcctgagccctggcaag
(SEQ ID NO: 60)
HGF-α
QRKRRNTIHEFKKSAKTTLIKIDPALKIKTKKVNTADQCANRCTRNKGLPFTCKAFVFDKARKQCL
WFPFNSMSSGVKKEFGHEFDLYENKDYIRNCIIGKGRSYKGTVSITKSGIKCQPWSSMIPHEHSFLPS
SYRGKDLQENYCRNPRGEEGGPWCFTSNPEVRYEVCDIPQCSEVECMTCNGESYRGLMDHTESGK
ICQRWDHQTPHRHKFLPERYPDKGFDDNYCRNPDGQPRPWCYTLDPHTRWEYCAIKTCADNTMN
DTDVPLETTECIQGQGEGYRGTVNTIWNGIPCQRWDSQYPHEHDMTPENFKCKDLRENYCRNPDG
SESPWCFTTDPNIRVGYCSQIPNCDMSHGQDCYRGNGKNYMGNLSQTRSGLTCSMWDKNMEDLH
RHIFWEPDASKLNENYCRNPDDDAHGPWCYTGNPLIPWDYCPISRCEGDTTPTIVNLDHPVISCAK
TKQLR (SEQ ID NO: 61)
HGF-β
VVNGIPTRTNIGWMVSLRYRNKHICGGSLIKESWVLTARQCFPSRDLKDYEAWLGIHDVHGRGDEK
CKQVLNVSQLVYGPEGSDLVLMKLARPAVLDDFVSTIDLPNYGCTIPEKTSCSVYGWGYTGLINYD
GLLRVAHLYIMGNEKCSQHHRGKVTLNESEICAGAEKIGSGPCEGDYGGPLVCEQHKMRMVLGVI
VPGRGCAIPNRPGIFVRVAYYAKWIHKIILTYKVPQS (SEQ ID NO: 62)
HCDR1 of anti-EGFR antibody
TYGMH (SEQ ID NO: 63)
HCDR2 of anti-EGFR antibody
VIWDDGSYKYYGDSVKG (SEQ ID NO: 64)
HCDR3 of anti-EGFR antibody
DGITMVRGVMKDYFDY (SEQ ID NO: 65)
LCDR1 of anti-EGFR antibody
RASQDISSALV (SEQ ID NO: 66)
LCDR2 of anti-EGFR antibody
DASSLES (SEQ ID NO: 67)
LCDR3 of anti-EGFR antibody
QQFNSYPLT (SEQ ID NO: 68)
VH of anti-EGFR antibody
QVQLVESGGGVVQPGRSLRLSCAASGFTFSTYGMHWVRQAPGKGLEWVAVIWDDGSYKYYGDS
VKGRFTISRDNSKNTLYLQMNSLRAEDTAVYYCARDGITMVRGVMKDYFDYWGQGTLVTVSS
(SEQ ID NO: 69)
VL of anti-EGFR antibody
AIQLTQSPSSLSASVGDRVTITCRASQDISSALVWYQQKPGKAPKLLIYDASSLESGVPSRFSGSESGT
DFTLTISSLQPEDFATYYCQQFNSYPLTFGGGTKVEIK (SEQ ID NO: 70)
Anti-c-Met polypeptide chain anti-c-Met-V_{H}H-Fc of V12
QVQLVESGGDSVQAGGSLRLSCAASAHAYSSGCMGWFRQAPGKGREGVATIYTRNDFTYYSDSVK
GRFTISRDDAKNTVYLQVNSLKAEDTAMYYCAAGSPFRYAGAWNVCGLKEEDYNYWGQGTLVT
VSSGGGGSGGGGSQVQLVESGGGLVQPGGSLRLSCAASGYSYSANCVGWFYQAPGKEREGVAAIN
TGGETATYADFVKGRFTISQDYAKNTVYLQMNSLKSEDTGMYYCAAVGTRKYWDECSLSQHAYK
NWGQGTLVTVSSGEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIE
KTISKAKGQPREPQVCTLPPSREEMTKNQVSLSCAVKGFYPSDIAVEWESNGQPENNYKTTPPVLDS
DGSFFLVSKLTVDKSRWQQGNVFSCSVMHEALHNRFTQKSLSLSPGK (SEQ ID NO: 71)
caagtgcagctggtcgagtccggcggcgactccgtgcaggctggcggatctctgagactgtcttgcgctgcttctgcccacgcttactcca
gcggctgcatgggctggttcagacaggcccctggcaagggcagagaaggagtggctaccatctacaccagaaacgactttacctattactc
cgattctgtcaagggcaggttcaccatctcccgggacgacgccaagaacaccgtgtacctgcaggtgaacagcctgaaggccgaggatacc
gccatgtactactgcgccgctggctcccctttccggtatgccggcgcctggaacgtgtgcggcctgaaagaagaagactacaactactggg
gccagggaaccttggtgaccgtctcatccgggggcggcggtagcggtggcggaggctctcaagtgcagctggtggaatctggcggaggact
ggttcagcctggcggctctctccggctgtcctgcgccgcctctggctactcctactctgccaactgcgtgggctggttctaccaggccccc
ggcaaggagagagagggcgtggccgccatcaacaccggcggcgagacagcaacctacgccgacttcgtgaaaggccggtttacaatctctc
aggattatgctaagaataccgtgtacctgcagatgaactccctgaagtccgaggacacaggcatgtactactgtgctgctgtgggcacccg
gaagtactgggacgagtgctctctgagccagcacgcctacaaaaattggggccaaggcaccctggtgaccgtgtccagtggcgaacccaag
tcctccgacaagacccacacctgccccccctgtcctgctccagagctgctgggcggcccttctgtgtttctgttccctcctaagcccaagg
acaccctgatgatctccagaacccctgaggtgacctgtgtggtggtggacgtgtctcacgaggatcctgaagtgaagttcaactggtacgt
ggacggcgtggaagtgcataacgccaagacaaagcctagagaggaacagtacaactctacctacagagtggtgtccgtgctgaccgtgctg
caccaggactggctgaacggcaaagagtacaagtgcaaggtgtccaacaaggctctgcctgcccctatcgagaagaccatcagcaaggcca
agggccagccccgcgagcctcaggtctgcaccctgcctccatccagagaagagatgaccaagaaccaggtttccctgtcttgtgctgtgaa
gggcttctaccctagcgacatcgccgtggaatgggagtctaatggccagcctgagaacaactacaagaccacacctcctgtgctcgactcc
gatggctccttcttcctggtgtccaagctgacggtggataagtctagatggcagcagggcaacgtgttctcctgctccgtgatgcacgagg
ccctgcataaccggttcacccagaagagcctgtccctgtctccaggaaaa (SEQ ID NO: 72)
Anti-c-Met polypeptide chain anti-c-Met-V_{H}H-Fc of V41
QVQLLESGGGSVQAGGSLRLSCAASGFTFSSGCMGWFRQAPGKGREGVATIYTRNDFTYYSDSVK
GRFTISRDNSKNTVYLQMNSLRAEDTAVYYCAAGSPFRYAGAWNVCGLKEEDYNYWGQGTLVTV
SSGGGGSGGGGSQVQLVESGGGSVQPGGSLRLSCAASGYSYSANCVGWFRQAPGKGREGVSAINT
GGETATYADFVKGRFTISRDNAKNTVYLQMNSLRAEDTAVYYCAAVGTRKYWDECSLSQHAYKN
WGQGTLVTVSSGEPKSSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEK
TISKAKGQPREPQVCTLPPSREEMTKNQVSLSCAVKGFYPSDIAVEWESNGQPENNYKTTPPVLDSD
GSFFLVSKLTVDKSRWQQGNVFSCSVMHEALHNRFTQKSLSLSPGK (SEQ ID NO: 73)
caggtgcagctgctggaatctggcggcggctctgtgcaggctggtggatccctgagactgtcttgtgctgcctccggctttaccttcagca
gtggctgcatgggctggtttagacaggcccccggcaaaggcagagaaggcgtggccaccatctacaccagaaacgacttcacctattattc
cgattccgtgaaaggcagattcaccatcagccgcgacaactccaaaaacaccgtgtacctgcagatgaactccctgagagccgaggatacc
gtgtgtactactgcgccgccggctctcctttccggtacgccggagcttggaacgtgtgtggcctgaaggaagaggactacaactactgggg
acaaggcaccctggtgaccgtgtctagcggcggaggtggctcgggcggaggcggctctcaagtgcagctggtggaatccggcggcggctcc
gtgcaacccggaggctccctgcggttgtcctgtgctgcctctggctactcctactccgccaactgcgtaggatggttcagacaggcccctg
gcaagggcagagagggcgtgtccgctatcaacaccggcggcgagacagccacctacgccgacttcgtgaagggccggttcaccatctctcg
ggacaacgccaagaacactgtctatctgcagatgaactccctgagagcagaagataccgccgtgtactactgtgctgctgtgggcacccgg
aagtactgggacgagtgctctctgtcccagcacgcctacaagaattggggccagggcacactagtcaccgtctcctctggcgagcctaagt
cctctgataagacccacacctgtcctccttgccctgctcctgagctgctgggcggcccttccgtcttcctgttccctccaaagcccaagga
cacactgatgatctccaggacacctgaagtgacctgcgtggtggtggacgtgtctcatgaggaccccgaggtgaagttcaactggtacgtg
gatggcgtggaagtgcacaatgccaagaccaagcctcgggaggaacagtacaactctacctacagagtggtgtccgtgctgaccgtcctgc
accaggactggctgaacggcaaagagtacaagtgcaaggtgtccaacaaggccctgccagctcctatcgagaagacaatcagcaaggccaa
gggccagcctagagagcctcaggtgtgcaccctgcctccttctcgggaagagatgaccaagaaccaggtgagcctgtcctgcgccgtgaaa
ggcttctacccctccgacatcgccgtcgagtgggagtctaatggacagcccgagaacaactacaagaccacccctcctgtgctggactccg
acggctctttcttcctggtttctaagctgaccgtggacaagtccagatggcagcagggcaacgtgttctcctgctccgtgatgcacgaagc
tctgcacaaccggtttacccagaagtctctgtctctcagcccaggcaag (SEQ ID NO: 74)
Anti-c-Met polypeptide chain anti-c-Met-V_{H}H-Fc of V35
QVQLLESGGGSVQAGGSLRLSCAASGHTYSSGCMGWFRQAPGKGREGVATIYTRNDFTYYSDSVK
GRFTISRDNSKNTVYLQMNSLRAEDTAVYYCAAGSPFRYAGAWNVCGLKEEDYNYWGQGTLVTV
SSGGGGSGGGGSQVQLVESGGGSVQPGGSLRLSCAASGYSYSANCVGWFRQAPGKGREGVSAINT
GGETATYADFVKGRFTISRDNAKNTVYLQMNSLRAEDTAVYYCAAVGTRKYWDECSLSQHAYKN
WGQGTLVTVSSGEPKSSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEK
TISKAKGQPREPQVCTLPPSREEMTKNQVSLSCAVKGFYPSDIAVEWESNGQPENNYKTTPPVLDSD
GSFFLVSKLTVDKSRWQQGNVFSCSVMHEALHNRFTQKSLSLSPGK (SEQ ID NO: 75)
caggttcagctgttggaatctggcggcggatctgttcaggctggcggatctctgagactgtcttgtgctgcttccggccacacctactcct
ctggctgtatgggatggttcagacaggcccctggcaagggaagagaaggcgtggccaccatctacacccggaacgacttcacctactactc
cgactccgtgaagggcagattcaccatcagccgggacaactccaagaacaccgtgtacctgcagatgaactccctgagagccgaggacacc
gccgtgtactattgtgccgctggcagcccctttagatacgctggcgcttggaacgtgtgcggcctgaaagaagaggactacaactactggg
gccagggcaccctggtcacagtttctagcggaggcggaggatcaggtggcggtggatctcaggtgcagttggttgaaagtggcggaggctc
tgtgcaaccaggcggaagcctcagattgtcttgcgccgcttctggctactcctacagcgccaattgtgtcggctggtttaggcaggctccc
ggaaaaggcagagaaggggtgtccgctatcaacaccggcggagagacagccacctacgccgatttcgtgaaaggccggtttaccatctctc
gcgataacgctaagaacacagtctatctccaaatgaacagcctgcgcgctgaggataccgctgtgtattactgcgctgccgtgggcaccag
aaagtactgggacgagtgctccctgagccagcacgcctataagaattggggacagggaaccctcgtgaccgtgtcctctggcgaacctaag
tcctccgacaagacccacacctgtcctccatgtcctgctccagaactgctcggcggaccttccgtgttcctgtttcctccaaagcctaagg
acaccctgatgatctctcggacccctgaagtgacctgcgtggtggtggatgtgtctcacgaggatcccgaagtgaagttcaattggtacgt
ggacggcgtggaagtgcacaacgccaagaccaagcctagagaggaacagtacaactccacctacagagtggtgtccgtgctgaccgtgctg
caccaggattggctgaacggcaaagagtacaagtgcaaggtgtccaacaaggccctgcctgctcctatcgaaaagaccatctccaaggcca
agggccagcctcgggaacctcaagtctgtaccctgcctcctagccgggaagagatgaccaagaaccaggtgtccctgtcctgcgctgtgaa
gggcttctacccttccgatatcgccgtggaatgggagagcaatggccagccagagaacaactacaagacaacccctcctgtgctggactcc
gacggctcattcttcctggtgtccaagctgacagtggacaagtccagatggcagcagggcaacgtgttctcctgctccgtgatgcacgagg
ccctgcacaacaggttcacccagaagtccctgtctctgagcccaggcaaa (SEQ ID NO: 76)
Anti-c-Met polypeptide chain anti-c-Met-V_{H}H-Fc of V42
QVQLLESGGGSVQAGGSLRLSCAASGHTYSSGCMGWFRQAPGKGREGVATIYTRNDFTYYSDTVK
GRFTISRDNSKNTVYLQMNSLRAEDTAVYYCAAGSPFRYAGAWNVCGLKEEDYNYWGQGTLVTV
SSGGGGSGGGGSQVQLVESGGGSVQPGGSLRLSCAASGYSYSANCVGWFRQAPGKGREGVSAINT
GGETATYADFVKGRFTISRDNAKNTVYLQMNSLRAEDTAVYYCAAVGTRKYWDECSLSQHAYKN
WGQGTLVTVSSGEPKSSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEK
TISKAKGQPREPQVCTLPPSREEMTKNQVSLSCAVKGFYPSDIAVEWESNGQPENNYKTTPPVLDSD
GSFFLVSKLTVDKSRWQQGNVFSCSVMHEALHNRFTQKSLSLSPGK (SEQ ID NO: 77)
caggttcagctgctggaatctggcggaggctccgtgcaggccggcggaagcctgagactgtcctgcgccgcctctggccatacatattctt
ccggctgcatgggctggttccggcaggctcctggcaagggcagggagggcgtggccacaatctacaccagaaacgacttcacctattattc
tgataccgtcaaaggcagattcaccatctctcgggacaactccaagaacaccgtgtacctgcagatgaactccctgcgggctgaggacacc
gccgtctactactgcgccgctggctctccttttagatacgctggcgcctggaacgtgtgtggcctgaaggaagaagactacaactactggg
gccaaggcaccctggtgaccgtgtcctccggcggcggaggctctggaggcggcggcagccaagtgcagctggtggaaagcggcggtggctc
cgtgcagccaggcggctctctgcggctgtcctgtgccgcttctggctactcctactccgccaactgcgtgggctggttcagacaggccccc
ggcaagggccgggaaggcgtgtctgccatcaacaccggcggcgagacagctacctacgccgacttcgtgaagggcagatttaccatctcca
gagataacgctaagaataccgtgtacctgcagatgaacagcctgcgggccgaggacaccgctgtgtactactgtgctgccgtgggcacccg
gaagtactgggacgagtgcagtctgagccagcacgcctacaagaattggggccagggaacactggtcaccgtgtccagtggagaacctaag
tcgtctgacaagacccacacctgtcctccatgccccgctcccgagctgctgggcggcccttctgtgttcctgttccctcctaagcccaagg
acacactgatgatcagcagaacccctgaagtgacctgcgtggtggtggacgtgtcccacgaggatcctgaggtgaagttcaactggtacgt
ggacggcgtggaagtgcacaacgccaagaccaagcctcgcgaggagcagtacaactccacctacagagtggtctctgtactgaccgtgctg
caccaggactggctgaacggaaaagagtacaagtgcaaggtgtccaacaaggctctgcctgcccctatcgagaagaccatctccaaggcca
aaggacagcctagagagcctcaagtgtgcaccctgcctccatctcgggaagagatgactaagaaccaggtctccctgtcctgtgccgtcaa
gggcttctacccctccgacatcgccgtggagtgggagtccaacggccagcctgagaacaactacaaaaccacacctcctgtgctggacagc
gatggctccttcttcctcgtgtccaagctgaccgttgataagtccagatggcagcagggcaacgtgttctcttgctccgtgatgcacgagg
ccctgcataatagatttacccagaagtctctgtcgctctctcccggcaaa (SEQ ID NO: 78)
Anti-c-Met polypeptide chain anti-c-Met-V_{H}H-Fc of V67
EVQLVESGGGSVQAQGSLRLSCTVSGSTYSSNFMAWFRQAPGKEREWVATITTGAGNTAYADSVK
GRFTISEDNAKRTAYLQMNSLKPEDTAVYYCAAGRGGFWRPAEYDFWGQGTQVTVSSGGGGSQV
QLVESGGGSVQPGGSLRLSCAASGYSYSANCVGWFRQAPGKGREGVSAINTGGETATYADFVKGR
FTISRDNAKNTVYLQMNSLRAEDTAVYYCAAVGTRKYWDECSLSQHAYKNWGQGTLVTVSSGEP
KSSDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQV
CTLPPSREEMTKNQVSLSCAVKGFYPSDIAVEWESNGQPENNYKTTPPVLDSDGSFFLVSKLTVDKS
RWQQGNVFSCSVMHEALHNRYTQKSLSLSPGK (SEQ ID NO: 79)
gaagtgcagctggtcgagtctggcggcggctctgtgcaggctcagggctccctgcggctgtcttgtaccgtttccgggtccacctactctt
ccaacttcatggcctggttccggcaggcccctggaaaagagagagaatgggtggctaccatcaccacaggcgctggcaacaccgcttatgc
cgactccgtcaagggcagattcaccatctctgaggacaacgccaagcggaccgcctacctgcagatgaacagcctgaagcctgaagatacc
gccgtgtactactgtgctgccggcagaggcggcttttggcggcccgctgagtacgacttctggggccaaggcactcaagtgacagtgtcct
ctggaggaggaggctctcaggtgcagctggtggaatctggcggcggctccgtgcaaccaggcggatctctgagactgtcctgcgctgcttc
cggctactcctactccgccaactgcgtgggctggttcagacaggcccctggcaaaggccgcgagggcgtctctgccatcaacaccggcgga
gagacagctacctacgccgattttgttaagggccggttcactatctccagagacaatgctaagaacaccgtgtacctgcagatgaactctc
tgagagccgaggacaccgctgtgtactactgcgccgctgtgggcacccggaagtactgggacgagtgctctctgtcccagcacgcctataa
gaattggggccagggcaccctcgtgaccgtgtcttcaggcgagcctaaaagcagcgataagacccacacctgccctccttgccccgctcct
gagctgctgggcggaccttctgtctttctgttcccacctaagcccaaggacaccctgatgatcagccggacacctgaggtgacctgtgtgg
tggtggacgtgtctcatgaggatcctgaggtgaagttcaactggtacgtggatggcgtggaagtgcacaatgccaagaccaagcctagaga
agagcagtacaactccacatacagagtggtctcggtgctgaccgtgctgcaccaggactggctgaacggcaaagaatacaagtgcaaggtg
tccaacaaggctctgcctgcccccatcgagaagacaatctccaaggccaagggccagcctcgggaacctcaggtgtgcaccctgcccccct
ccagggaagaaatgacaaagaaccaggtgtccctatcctgtgccgtgaaaggcttctacccttccgacatcgccgtggagtgggagtccaa
cggccagcccgagaacaactacaaaaccacccctccagtgctggactctgacggctccttcttcctggtgagcaagctgaccgtggacaag
tctagatggcaacagggaaacgtgttcagctgctccgtcatgcacgaggccctgcacaacagatacacccagaagtccctgagcctgtctc
ctggcaag (SEQ ID NO: 80)
Anti-c-Met polypeptide chain anti-c-Met-V_{H}H-Fc of V73
EVQLVESGGGSVQAGGSLRLSCAVSGFTVSSNFMAWFRQAPGKGREWVSTITTGAGNTAYADSVK
GRFTISEDNSKNTLYLQMNSLRAEDTAVYYCAAGRGGFWRPAEYDFWGQGTTVTVSSGGGGSQV
QLVESGGGSVQPGGSLRLSCAASGYSYSANCVGWFRQAPGKGREGVSAINTGGETATYADFVKGR
FTISRDNAKNTVYLQMNSLRAEDTAVYYCAAVGTRKYWDECSLSQHAYKNWGQGTLVTVSSGEP
KSSDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQV
CTLPPSREEMTKNQVSLSCAVKGFYPSDIAVEWESNGQPENNYKTTPPVLDSDGSFFLVSKLTVDKS
RWQQGNVFSCSVMHEALHNRYTQKSLSLSPGK (SEQ ID NO: 81)
gaggtgcagctggtggaatcaggcggaggctccgtgcaggccggcggctctttgagactgtcttgtgccgtgtccggcttcaccgtgtctt
ctaacttcatggcctggttcagacaggctcctggcaagggcagagagtgggtcagcaccatcaccaccggcgctggaaacaccgcctacgc
cgactccgtgaaagggcgctttacaatcagcgaggacaactccaagaataccctgtacctgcagatgaactctctgcgggccgaggatacc
gccgtgtactactgtgctgctggcagaggcggcttttggcggccagctgagtacgacttctggggccaaggcaccacagtcaccgtgtcct
ctggcggaggtggcagccaagtgcagctggtcgaatctggaggtggctccgttcagcctggcggctctctgagactgtcttgcgccgcttc
cggctactcttactccgccaactgcgtgggctggttccggcaggcccccggcaaaggcagagaaggcgtgagcgccatcaacaccggcggc
gaaaccgctacctatgccgatttcgtgaagggaagattcaccatctctcgggacaacgccaagaacaccgtgtatctgcagatgaactctc
tgagagccgaggatacagctgtgtactactgcgctgctgtgggcaccagaaagtactgggacgagtgctccctgtcccagcacgcctataa
gaactggggacagggcacactcgtgaccgtgtctagcggcgagcctaaaagctctgacaaaacccacacctgtcctccttgccccgctcct
gagctgctgggcggcccttccgtgtttctgttccctcccaagcccaaggacacactgatgatctccagaaccccggaagtgacctgcgtgg
tggtggacgtgtctcacgaggaccctgaggtcaagttcaactggtacgtggatggcgtggaagtgcataatgctaagaccaagcctcggga
ggaacagtacaactccacctacagagtggtttccgtgctgaccgtgctgcaccaagactggctgaacggcaaagagtacaagtgcaaggtg
tccaacaaggctctgcctgcccccatcgagaagacgatctccaaggccaagggccagcctagggaacctcaggtgtgcaccctgcctcctt
cccgggaagagatgaccaaaaaccaggtcagcctgtcttgcgccgtgaagggcttctacccctccgacatcgccgtggaatgggagtccaa
cggccagcccgagaataactacaagaccacccctcctgtgctcgatagcgacggctccttcttcctggtgtctaagctgacagtggacaag
tctagatggcagcagggcaacgtgttctcctgttccgtgatgcacgaggccctgcacaaccggtacacccagaagtccctgagcctgtctc
caggcaag (SEQ ID NO: 82)
Anti-c-Met polypeptide chain anti-c-Met-V_{H}H-Fc of V74
EVQLVESGGGSVQAGGSLRLSCAVSGFTVSSNFMAWFRQAPGKGREWVSTITTGAGNTAYADSVK
GRFTISEDNSKNTAYLQMNSLRAEDTAVYYCAAGRGGFWRPAEYDFWGQGTTVTVSSGGGGSQV
QLVESGGGSVQPGGSLRLSCAASGYSYSANCVGWFRQAPGKGREGVSAINTGGETATYADFVKGR
FTISRDNAKNTVYLQMNSLRAEDTAVYYCAAVGTRKYWDECSLSQHAYKNWGQGTLVTVSSGEP
KSSDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQV
CTLPPSREEMTKNQVSLSCAVKGFYPSDIAVEWESNGQPENNYKTTPPVLDSDGSFFLVSKLTVDKS
RWQQGNVFSCSVMHEALHNRYTQKSLSLSPGK (SEQ ID NO: 83)
gaagtgcagctggtggaatccggcggcggatctgtgcaggccgggggctccctgagactgtcttgcgccgtgtccggcttcaccgtgtctt
ctaacttcatggcctggtttcgccaggcacctggcaagggcagagagtgggtgtctaccatcaccaccggcgctgggaacaccgcttacgc
cgattctgtgaaaggccggttcaccatctccgaggataattccaagaacacagcttatctgcagatgaacagcctgagagccgaggacacc
gccgtatactactgtgctgctggcagaggcggcttttggcggcctgctgagtacgacttctggggccagggcaccacagtcacagtttcct
ctggaggcggtggctctcaagtgcagctggtggagtctgggggaggctctgtccaacctggaggatctctgcggctgagctgcgccgcctc
tggctactcctacagcgccaactgcgtgggctggttcagacaggcccctggaaagggcagagaaggcgtttctgccatcaacaccggcggc
gagacagccacatacgccgacttcgtgaagggcagattcaccatcagtcgggacaacgccaaaaacaccgtgtacctgcagatgaactccc
tccgggccgaggacaccgctgtgtactactgcgctgctgtcggcaccagaaagtactgggacgagtgctctctgagccagcacgcctacaa
aaattggggccaaggcaccctcgtgacagtgagcagcggcgaacccaagtccagcgacaagacccacacctgccctccttgtcctgcccca
gagctgctgggcggcccttccgtgtttctgttcccacccaagcctaaggacactctgatgatcagcagaacccctgaggtgacctgtgtgg
tggtggacgtgtctcacgaagatcctgaagtcaagttcaactggtacgtggacggcgtggaagtgcacaacgctaagaccaagcccagaga
agagcagtacaacagcacctacagagtggtctccgtgctgaccgtgctgcaccaggactggctgaacggcaaagagtataagtgcaaggtg
tccaacaaggctctgcccgctcctatcgagaagaccatctccaaggccaagggccagcctcgggagcctcaggtgtgcaccctgcctccct
cccgggaagagatgaccaagaaccaggtgtccttgtcttgtgctgtgaagggcttctacccttcggacatcgccgtggaatgggagtccaa
tggacagcccgagaacaactacaagacaacccctcctgtgctggactccgatggctccttcttcctggtgtccaagctgaccgtggataag
tctaggtggcagcagggcaacgtgttctcctgctccgtgatgcacgaggccctgcataacagatacacccagaagtccctgtccctgtctc
ctggcaaa (SEQ ID NO: 84)
Heavy chain anti-EGFR-HC-Fc of anti-EGFR antibody
QVQLVESGGGVVQPGRSLRLSCAASGFTFSTYGMHWVRQAPGKGLEWVAVIWDDGSYKYYGDS
VKGRFTISRDNSKNTLYLQMNSLRAEDTAVYYCARDGITMVRGVMKDYFDYWGQGTLVTVSSAS
TKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWCLVKGFYPSDIAVEWES
NGQPENNYKTTPPVLDSDGSFFLYSKLTVDKSRWQQGNVFSCSVMHEALHNHYTQKSLSLSPGK
(SEQ ID NO: 85)
caggtgcagctggtggagtccggcggcggagtggtgcagccaggaagatccctgcggctgagctgtgccgcttccggcttcaccttctcca
cctacggcatgcactgggtgcggcaggcccccggaaagggactggagtgggtggctgtgatctgggacgacggctcctacaagtattacgg
cgattccgtgaagggccggttcaccatctcccgggacaactccaagaacaccctgtacctgcagatgaatagcctgagggccgaggacacc
gctgtgtactactgtgcccgggacggcatcaccatggtgcggggagtgatgaaggattacttcgactactggggccagggcaccctggtga
ccgtgagctccgccagtaccaaaggcccttccgtgtttcctctggccccatcctccaagagtacatctggaggcaccgctgctctgggctg
cctggtgaaggactacttccccgagcccgtgaccgtcagctggaactccggcgccctgacctctggcgttcatacctttcccgccgttctg
cagagctctggcctgtacagcctgtcttccgtcgtgaccgtgccttctagctctctgggaacacagacctacatctgcaacgtgaaccaca
agcccagcaacaccaaggggacaagcgggtggaacccaagtcctgcgacaaaacccacacctgtcctccttgtccagcccctgagctgctg
ggcggcccttctgtctttctgttccctcctaagcctaaggacacgctcatgatctcccggacacctgaagtgacctgcgtggtggtggatg
tgtctcacgaggaccctgaggtgaagttcaactggtacgtggacggcgtggaagtccataacgccaagaccaagcctagagaggagcagta
caattccacctatagggtggtgagcgtgctgaccgtgctgcaccaggattggctgaatggcaaggagtacaagtgcaaggtgagcaataag
gctctgcctgctcccatcgagaagaccatctccaaggctaagggccagcctagggagccccaggtgtataccctgcctccctgccgggagg
agatgaccaagaaccaggtgtccctgtggtgcctggtgaagggcttctacccttccgacatcgctgtggagtgggagtccaatggccagcc
cgagaataattacaagaccacccctcccgtgctggattccgacggctccttcttcctgtactccaagctgaccgtggacaagagcaggtgg
cagcagggcaacgtgttcagctgtagcgtgatgcacgaggccctgcacaaccactacacccagaagtccctgagcctgagccccggcaag
(SEQ ID NO: 86)
Light chain anti-EGFR-LC of anti-EGFR antibody
AIQLTQSPSSLSASVGDRVTITCRASQDISSALVWYQQKPGKAPKLLIYDASSLESGVPSRFSGSESGT
DFTLTISSLQPEDFATYYCQQFNSYPLTFGGGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSS
PVTKSFNRGEC (SEQ ID NO: 87)
gctatccagctgacccagtcccctagctccctgagcgcctccgtgggcgacagggtgaccatcacctgcagggccagccaggatatctcct
ccgccctggtgtggtaccagcagaagcccggcaaggcccccaagctgctgatctatgacgctagctccctggagtccggcgtgccctccag
gttcagcggctccgagtccggcaccgacttcaccctgaccatctccagcctgcagcccgaggacttcgccacctattattgtcagcagttc
aatagctaccccctgaccttcggggcggcaccaaggtggagatcaagaggaccgtggccgcccctagcgtgttcatcttccctcctagcga
cgagcagctgaagagcggcaccgcctccgtggtgtgcctgctgaacaacttctatcccagggaggctaaggtgcagtggaaggtggataac
gccctgcagtccggcaatagccaggagagcgtgaccgagcaggattccaaggattccacctactccctgagcagcaccctgaccctgagca
aggctgattacgagaagcacaaggtgtatgcttgtgaggtgacccaccagggcctgagcagccccgtgaccaagtccttcaacaggggcga
gtgt (SEQ ID NO: 88)
First hinge
GEPKSSDKTHTCPPCP (SEQ ID NO: 89)
Second hinge
EPKSCDKTHTCPPCP (SEQ ID NO: 90)
Peptide linker
GGGGS (SEQ ID NO: 91)
Heavy chain constant region of human IgG1
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPGK
(SEQ ID NO: 92)

All patents, patent applications, and other identified publications are explicitly incorporated herein by reference for the purpose of description and disclosure. These publications are provided solely because they were disclosed prior to the filing date of the present disclosure. All statements as to the dates of these documents or descriptions as to the contents of these documents are based on the information available to the applicant and do not constitute any admission as to the correctness of the dates or the content of these documents. Moreover, in any country or region, any reference to these publications herein should not be construed as an admission that the publications form part of the commonly recognized knowledge in the art.

Although the present disclosure has been described in detail herein above through general explanations and specific embodiments, it will be apparent to those skilled in the art that modifications or improvements can be made based on the present disclosure. Accordingly, such modifications and improvements made without departing from the spirit of the present disclosure all fall within the claimed scope of the present disclosure.

1.-20. (canceled)

21. An antibody-drug conjugate, a stereoisomer thereof, a pharmaceutically acceptable salt thereof, or a solvate of the antibody-drug conjugate, the stereoisomer thereof, or the pharmaceutically acceptable salt thereof, comprising a multispecific antibody, wherein the multispecific antibody comprises
(i) a first antigen-binding portion binding to a first antigen;
(ii) a second antigen-binding portion binding to a second antigen; and
(iii) a third antigen-binding portion binding to the first antigen,
wherein the first antigen is c-Met, and the second antigen is EGFR;
wherein the third antigen-binding portion is a single variable domain and comprises a complementarity determining region (CDR) 1 comprising the amino acid sequence shown in SEQ ID NO: 4, a CDR2 comprising the amino acid sequence shown in SEQ ID NO: 5 and wherein X₁ is T, and a CDR3 comprising the amino acid sequence shown in SEQ ID NO: 6;
wherein the first antigen-binding portion is a single variable domain and comprises a CDR1 comprising the amino acid sequence shown in SEQ ID NO: 7, a CDR2 comprising the amino acid sequence shown in SEQ ID NO: 8, and a CDR3 comprising the amino acid sequence shown in SEQ ID NO: 9; and
wherein the second antigen-binding portion comprises a heavy chain CDR (HCDR) 1 comprising the amino acid sequence shown in SEQ ID NO: 63, a HCDR2 comprising the amino acid sequence shown in SEQ ID NO: 64, and a HCDR3 comprising the amino acid sequence shown in SEQ ID NO: 65, a light chain CDR (LCDR) 1 comprising the amino acid sequence shown in SEQ ID NO: 66, a LCDR2 comprising the amino acid sequence shown in SEQ ID NO: 67, and a LCDR3 comprising the amino acid sequence shown in SEQ ID NO: 68.

22. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21,
wherein the first antigen-binding portion comprises an amino acid sequence having at least 85% identity to the amino acid sequence shown in SEQ ID NO: 36; and/or
wherein the third antigen-binding portion comprises an amino acid sequence having at least 85% identity to the amino acid sequence shown in SEQ ID NO: 35.

23. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21,
wherein the first antigen-binding portion comprises the amino acid sequence shown in SEQ ID NO: 36, and the third antigen-binding portion comprises the amino acid sequence shown in SEQ ID NO: 35.

24. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21,
wherein the second antigen-binding portion comprises a heavy chain variable region having an amino acid sequence having at least 85% identity to the amino acid sequence shown in SEQ ID NO: 69, and a light chain variable region having an amino acid sequence having at least 85% identity to the amino acid sequence shown in SEQ ID NO: 70.

25. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21,
wherein the heavy chain variable region of the second antigen-binding portion comprises the amino acid sequence shown in SEQ ID NO: 69, and the light chain variable region of the second antigen-binding portion comprises the amino acid sequence shown in SEQ ID NO: 70.

26. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21,
wherein the single variable domain is derived from a camelid or is humanized; and/or
wherein the second antigen-binding portion is a Fab, an scFv, or an scFab; and/or
wherein the second antigen-binding portion is murine, chimeric, or humanized.

27. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21,
wherein the third antigen-binding portion and the first antigen-binding portion are fused to each other; or
wherein the third antigen-binding portion and the first antigen-binding portion are fused to each other via a peptide linker; and/or
wherein the third antigen-binding portion, at the C-terminus, is fused to the N-terminus of the first antigen-binding portion.

28. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21,
wherein the multispecific antibody further comprises:
(iv) an Fc domain composed of two Fc polypeptides.

29. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 28,
wherein the Fc domain is an IgG Fc domain.

30. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 28,
wherein the Fc domain comprises an amino acid substitution that promotes association of the two Fc polypeptides of the Fc domain; and/or
wherein the Fc domain comprises an amino acid substitution that reduces or eliminates binding of a CH3 region of one Fc polypeptide in the Fc domain to Protein A.

31. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 30,
wherein according to the EU numbering, one of the Fc polypeptides of the Fc domain comprises amino acid substitutions 349C, 366S, 368A, 407V, 435R, and 436F, and the other Fc polypeptide comprises amino acid substitutions 354C and 366W; or,
wherein according to the EU numbering, one of the Fc polypeptides of the Fc domain comprises amino acid substitutions 349C, 366S, 368A, 407V, and 435R, and the other Fc polypeptide comprises amino acid substitutions 354C and 366W.

32. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 28,
wherein:
one polypeptide chain comprises an amino acid sequence having at least 85% identity to the amino acid sequence shown in SEQ ID NO: 77,
another polypeptide chain comprises an amino acid sequence having at least 85% identity to the amino acid sequence shown in SEQ ID NO: 85, and
the other polypeptide chain comprises an amino acid sequence having at least 85% identity to the amino acid sequence shown in SEQ ID NO: 87.

33. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 28,
wherein:
one polypeptide chain comprises the amino acid sequence shown in SEQ ID NO: 77,
another polypeptide chain comprises the amino acid sequence shown in SEQ ID NO: 85, and
the other polypeptide chain comprises the amino acid sequence shown in SEQ ID NO: 87.

34. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21,
wherein the antibody-drug conjugate is formed by linking a drug-linker having a structure represented by the following formula Ia 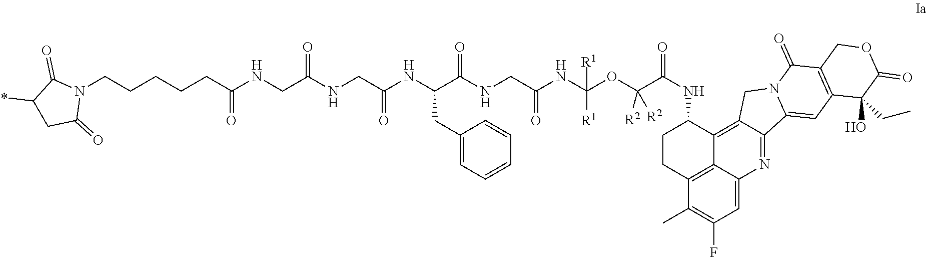
to the multispecific antibody, and the drug-linker is linked to the multispecific antibody at
a position indicated by * in the structure represented by formula Ia, wherein
R¹ and R² are each independently selected from a hydrogen atom and a deuterium atom.

35. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 34,
wherein R¹ is a hydrogen atom and R² is a deuterium atom.

36. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 34,
wherein a DAR of the antibody-drug conjugate is 2 to 8; or
wherein the DAR of the antibody-drug conjugate is 6.

37. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 33,
wherein the antibody-drug conjugate is formed by linking a drug-linker having a structure represented by the following formula Ia-1 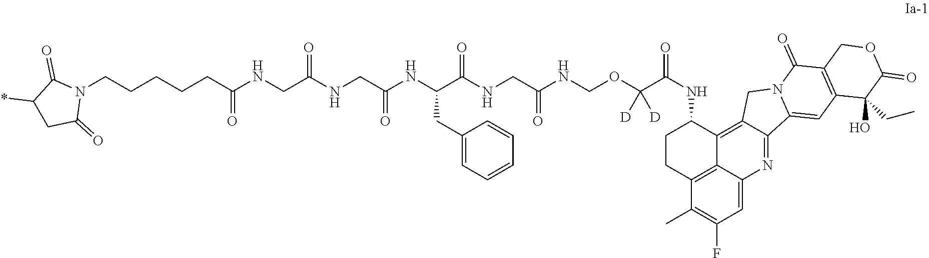 [SMILES: *C1CC(=O)N(CCCCCC(=O)NCC(=O)NCC(=O)N[C@@H](Cc2ccccc2)C(=O)NCC(=O)NCOC([2H])([2H])C(=O)N[C@H]2CCc3c(C)c(F)cc4nc5c(c2c34)Cn2c-5cc3c(c2=O)COC(=O)[C@]3(O)CC)C1=O]
to the multispecific antibody, and the drug-linker is linked to the multispecific antibody at a position indicated by * in the structure represented by formula Ia-1.

38. The antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 37,
wherein a DAR of the antibody-drug conjugate is 6.

39. A pharmaceutical composition, comprising the antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21, wherein the pharmaceutical composition further comprises a pharmaceutically acceptable excipient.

40. A method for treating a disease expressing c-Met and/or EGFR, comprising administering to a subject the antibody-drug conjugate, the stereoisomer thereof, the pharmaceutically acceptable salt thereof, or the solvate of the antibody-drug conjugate, the stereoisomer thereof, or the pharmaceutically acceptable salt thereof according to claim 21.

41. The method according to claim 40, wherein the disease expressing c-Met and/or EGFR is epithelial cell carcinoma, squamous cell carcinoma, glioblastoma, breast cancer, ovarian cancer, lung cancer, lung adenocarcinoma, colorectal cancer, anal cancer, prostate cancer, kidney cancer, liver cancer, bladder cancer, head and neck cancer, gastric cancer, pancreatic cancer, skin cancer, oral cancer, pharyngeal cancer, nasal cancer, tongue cancer, esophageal cancer, testicular cancer, vaginal cancer, cervical cancer, spleen cancer, testicular cancer, thyroid cancer, salivary gland cancer, and/or thymus cancer.
